SEQUENCE LISTING (75 sequences)

SEQ ID NO: 1 - DNA, 36 bp, synthetic construct
  misc_feature 1..36: note = Description of Artificial Sequence: Synthetic primer
  modified_base 24: mod_base = OTHER; note = Methoxy base
  modified_base 32..36: mod_base = OTHER; note = Methoxy base
  source 1..36: mol_type = other DNA
gactcgatatcgagtcgcttccacagttatctaccg

SEQ ID NO: 2 - DNA, 33 bp, synthetic construct
  misc_feature 1..33: note = Description of Artificial Sequence: Synthetic primer
  modified_base 24: mod_base = OTHER; note = Methoxy base
  modified_base 29..33: mod_base = OTHER; note = Methoxy base
  source 1..33: mol_type = other DNA
gactcgatatcgagtcgaaatgcaacgacacct

SEQ ID NO: 3 - DNA, 26 bp, synthetic construct
  misc_feature 1..26: note = Description of Artificial Sequence: Synthetic probe
  source 1..26: mol_type = other DNA
gctacacgactttygctgaaggtagc

SEQ ID NO: 4 - DNA, 26 bp, synthetic construct
  misc_feature 1..26: note = Description of Artificial Sequence: Synthetic probe
  misc_feature 1: note = CALRed610nm-modified
  misc_feature 26: note = BHQ2-modified
  source 1..26: mol_type = other DNA
gctacacgactttygctgaaggtagc

SEQ ID NO: 5 - DNA, 26 bp, synthetic construct
  misc_feature 1..26: note = Description of Artificial Sequence: Synthetic probe
  misc_feature 1: note = FAM or FITC-modified
  misc_feature 26: note = BHQ1-modified
  source 1..26: mol_type = other DNA
gctacacgactttygctgaaggtagc

SEQ ID NO: 6 - DNA, 36 bp, synthetic construct
  misc_feature 1..36: note = Description of Artificial Sequence: Synthetic primer
  modified_base 24: mod_base = OTHER; note = Methoxy base
  modified_base 30..36: mod_base = OTHER; note = Methoxy base
  source 1..36: mol_type = other DNA
gactcgatatcgagtctgactagcggaggctagaag

SEQ ID NO: 7 - DNA, 36 bp, synthetic construct
  misc_feature 1..36: note = Description of Artificial Sequence: Synthetic primer
  modified_base 24: mod_base = OTHER; note = Methoxy base
  modified_base 30..36: mod_base = OTHER; note = Methoxy base
  source 1..36: mol_type = other DNA
gactcgatatcgagtctgactagcagaggctagaag

SEQ ID NO: 8 - DNA, 33 bp, synthetic construct
  misc_feature 1..33: note = Description of Artificial Sequence: Synthetic primer
  modified_base 24: mod_base = OTHER; note = Methoxy base
  modified_base 28..33: mod_base = OTHER; note = Methoxy base
  source 1..33: mol_type = other DNA
gactcgatatcgagtctattgacgctctcgcac

SEQ ID NO: 9 - DNA, 33 bp, synthetic construct
  misc_feature 1..33: note = Description of Artificial Sequence: Synthetic primer
  modified_base 24: mod_base = OTHER; note = Methoxy base
  modified_base 28..33: mod_base = OTHER; note = Methoxy base
  source 1..33: mol_type = other DNA
gactcgatatcgagtctactgacgctctcgcac

SEQ ID NO: 10 - DNA, 27 bp, synthetic construct
  misc_feature 1..27: note = Description of Artificial Sequence: Synthetic probe
  source 1..27: mol_type = other DNA
cgcaagggagagagatgggtgcttgcg

SEQ ID NO: 11 - DNA, 36 bp, synthetic construct
  misc_feature 1..36: note = Description of Artificial Sequence: Synthetic primer
  modified_base 24: mod_base = OTHER; note = Methoxy base
  modified_base 32..36: mod_base = OTHER; note = Methoxy base
  source 1..36: mol_type = other DNA
gactcgatatcgagtccaaaaacagcatattgacgc

SEQ ID NO: 12 - DNA, 34 bp, synthetic construct
  misc_feature 1..34: note = Description of Artificial Sequence: Synthetic primer
  modified_base 24: mod_base = OTHER; note = Methoxy base
  modified_base 30..34: mod_base = OTHER; note = Methoxy base
  source 1..34: mol_type = other DNA
gactcgatatcgagtcagacagcaggatctctgg

SEQ ID NO: 13 - DNA, 34 bp, synthetic construct
  misc_feature 1..34: note = Description of Artificial Sequence: Synthetic primer
  modified_base 24: mod_base = OTHER; note = Methoxy base
  modified_base 30..34: mod_base = OTHER; note = Methoxy base
  source 1..34: mol_type = other DNA
gactcgatatcgagtcagacagcaggatctgtgg

SEQ ID NO: 14 - DNA, 26 bp, synthetic construct
  misc_feature 1..26: note = Description of Artificial Sequence: Synthetic probe
  source 1..26: mol_type = other DNA
cgcatctggtctttcccagcgatgcg

SEQ ID NO: 15 - DNA, 37 bp, synthetic construct
  misc_feature 1..37: note = Description of Artificial Sequence: Synthetic primer
  modified_base 24: mod_base = OTHER; note = Methoxy base
  modified_base 33..37: mod_base = OTHER; note = Methoxy base
  source 1..37: mol_type = other DNA
gactcgatatcgagtcaaatgcagatggtctcagcta

SEQ ID NO: 16 - DNA, 37 bp, synthetic construct
  misc_feature 1..37: note = Description of Artificial Sequence: Synthetic primer
  modified_base 24: mod_base = OTHER; note = Methoxy base
  modified_base 33..37: mod_base = OTHER; note = Methoxy base
  source 1..37: mol_type = other DNA
gactcgatatcgagtcaaatgcaaatggtctcagcta

SEQ ID NO: 17 - DNA, 37 bp, synthetic construct
  misc_feature 1..37: note = Description of Artificial Sequence: Synthetic primer
  modified_base 24: mod_base = OTHER; note = Methoxy base
  modified_base 33..37: mod_base = OTHER; note = Methoxy base
  source 1..37: mol_type = other DNA
gactcgatatcgagtcaaatgcagatggtttcagcta

SEQ ID NO: 18 - DNA, 39 bp, synthetic construct
  misc_feature 1..39: note = Description of Artificial Sequence: Synthetic primer
  modified_base 24: mod_base = OTHER; note = Methoxy base
  modified_base 35..39: mod_base = OTHER; note = Methoxy base
  source 1..39: mol_type = other DNA
gactcgatatcgagtcctccttttcccattccattcatt

SEQ ID NO: 19 - DNA, 39 bp, synthetic construct
  misc_feature 1..39: note = Description of Artificial Sequence: Synthetic primer
  modified_base 24: mod_base = OTHER; note = Methoxy base
  modified_base 35..39: mod_base = OTHER; note = Methoxy base
  source 1..39: mol_type = other DNA
gactcgatatcgagtcctccctttcccattccattcatt

SEQ ID NO: 20 - DNA, 39 bp, synthetic construct
  misc_feature 1..39: note = Description of Artificial Sequence: Synthetic primer
  modified_base 24: mod_base = OTHER; note = Methoxy base
  modified_base 35..39: mod_base = OTHER; note = Methoxy base
  source 1..39: mol_type = other DNA
gactcgatatcgagtcctcctttccccattccattcatt

SEQ ID NO: 21 - DNA, 28 bp, synthetic construct
  misc_feature 1..28: note = Description of Artificial Sequence: Synthetic probe
  source 1..28: mol_type = other DNA
gccaagctatgaacacagcaaacttggc

SEQ ID NO: 22 - DNA, 39 bp, synthetic construct
  misc_feature 1..39: note = Description of Artificial Sequence: Synthetic primer
  modified_base 24: mod_base = OTHER; note = Methoxy base
  modified_base 33..39: mod_base = OTHER; note = Methoxy base
  source 1..39: mol_type = other DNA
gactcgatatcgagtcggcccactgtattgctactgaaa

SEQ ID NO: 23 - DNA, 39 bp, synthetic construct
  misc_feature 1..39: note = Description of Artificial Sequence: Synthetic primer
  modified_base 24: mod_base = OTHER; note = Methoxy base
  modified_base 33..39: mod_base = OTHER; note = Methoxy base
  source 1..39: mol_type = other DNA
gactcgatatcgagtcggcccactgcactgctactaaaa

SEQ ID NO: 24 - DNA, 36 bp, synthetic construct
  misc_feature 1..36: note = Description of Artificial Sequence: Synthetic primer
  modified_base 24: mod_base = OTHER; note = Methoxy base
  modified_base 30..36: mod_base = OTHER; note = Methoxy base
  source 1..36: mol_type = other DNA
gactcgatatcgagtctgtgatcaactccatgtgcc

SEQ ID NO: 25 - DNA, 28 bp, synthetic construct
  misc_feature 1..28: note = Description of Artificial Sequence: Synthetic probe
  source 1..28: mol_type = other DNA
cgctacatctctgctgtacatggtagcg

SEQ ID NO: 26 - DNA, 18958 bp, Zaire ebolavirus
  source 1..18958: mol_type = other DNA
cggacacacaaaaagaaagaagaatttttaggatcttttgtgtgcgaataactatgaggaagattaataattttcctctcattgaaatttatatcggaatttaaattgaaattgttactgtaatcacacctggtttgtttcagagccacatcacaaagatagagaacagcctaggtctccgaagggaacaagggcaccagtgtgctcagttgaaaatcccttgtcaacatctaggtcttatcacatcacaagttccacctcagactctgcagggtgatccaacaaccctaatagaaaaattattgttaacggacagcattagttcacagtcaaacaagcaagattgagaattaaccttgattttgaacttcaacacctagaggattggagattcaacaaccctaaaacttggggtaaaacattggaaatagttgaaagacaaattgctcggaatcacaaaattccgagtatggattctcgtcctcagaaagtctggatgacgccgagtcttactgaatctgacatggattaccacaagatcttgacagcaggtctgtccgttcaacaggggattgttcggcaaagagtcatcccagtgtatcaagtaaacaatcttgaggaaatttgccaacttatcatacaggcctttgaagcaggtgttgattttcaagagagtgcggacagtttccttctcatgctttgtcttcatcatgcgtaccaaggagatcacaaacttttcttggaaagtggtgcagtcaagtatttggaagggcacgggttccgttttgaagtcaagaaacgtgatggggtgaagcgccttgaggaattgctgccagcagtatctagtggaaaaaacattaagagaacacttgctgccatgccggaagaggagacgactgaagctaatgccggtcagtttctctcttttgcaagtctattccttccgaaattggtagtaggagaaaaggcttgccttgagaaagttcaaaggcaaattcaagtacatgcagagcaaggactgatacaatatccaacagcttggcaatcagtaggacacatgatggtgattttccgtttgatgcgaacaaattttttgatcaaatttctcctaatacaccaagggatgcacatggttgccgggcatgatgccaacgatgctgtgatttcaaattcagtggctcaagctcgtttttcaggtttattgattgtcaaaacagtccttgatcatatcctacaaaagacagaacgaggagttcgtctccatcctcttgcaaggactgccaaggtaaaaaatgaggtgaactcctttaaggctgcactcagctccctggccaagcatggagagtatgctcctttcgcccgacttttgaacctttctggagtaaataatcttgagcatggtcttttccctcaactatcggcaattgcactcggagtcgccacagcacacgggagcaccctcgcaggagtaaatgttggagaacagtatcaacagctcagagaggctgccactgaagctgagaagcaactccaacaatatgcagaatctcgcgaacttgaccatcttggacttgatgatcaggaaaagaaaattcttatgaacttccatcagaaaaagaacgaaatcagcttccagcaaacaaacgctatggtaactctaagaaaagagcgcctggccaagctgacagaagctatcactgctgcatcactgcccaaaacaagtggaccttacgatgatgatgacgacattccctttccaggacccatcaatgatgacgacaatcctggccatcaagatgatgatccgactgactcacaggatacgaccattcccgatgtggtggttgatcccgatgatggaagctacggcgaataccagagttactcggaaaacggcatgaatgcaccagatgacttggtcctattcgatctagacgaggacgacgaggacactaagccagtgcctaacagattgaccaagggtggacaacagaaaaacagtcaaaagggccagcatacagagggcagacagacacaatccaggccaactcaaaatgtcccaggccctcgcagaacaatccaccacgccagtgctccactcacggacaacgacagaggaaatgaaccctccggctcaaccagccctcgcatgctgacaccaattaacgaagaggcagacccactggacgatgccgacgacgagacgtctagtcttccgcccttggagtcagacgatgaagaacaggacagggacgaaacttccaaccgcacacccactgtcgccccaccggctcccgtatacagagatcactctgaaaagaaagaactcccgcaagatgagcagcaagatcaggaccacactcaagaggccaggaaccaggacagtgacaacacccagccagaacactcttttgaggagatgtatcgccacattctaagatcacagggaccatttgatgctgttttgtattatcatatgatgaaggatgagcctgtagttttcagtactagtgatggcaaagagtacacgtatccggactcccttgaagaggaatatccaccatggctcactgaaaaagaggccatgaatgaagagaatagatttgttacattggatggtcaacaattttattggccggtaatgaatcacaagaataaattcatggcaatcctgcaacatcatcagtgaatgagaatggaataatgggatgatttaaccgacaaatagctaacattaaatagtcaagaaacgcaaacaggaagaatttttgatgtctaaggtgtgaattattatcacaataaaagtgattcttatttttgaatttaaagctagcttattattactagccgtttttcaaagttcaatttgagtcttaatgcaaataggcgttaagccacagttatagccataattgtaactcaatatcttagctagcgatttatctaaattaaattacattatgcttttataacttacctactagcctgcccaacatttacacgatcgttttataattaagaaaaaactaatgatgaagattaaaaccttcatcatccttacgtcaattgaattctctagcactcgaagcttattgtcttcaatgtaaaagaaaagctggtccaacaagatgacaactagaacaaagggcaggggccatactgtggccacgactcaaaacgacagaatgccaggccctgagctttcgggctggatctccgagcagctaatgaccggaagaattcctgtaagcgacatcttctgtgatattgagaacaatccaggattatgttacgcatcccaaatgcaacaaacaaagccaaacccgaagatgcgcaacagtcaaacccaaacggacccaatttgcaatcatagttttgaggaggtagtacaaacattggcttcattggctactgttgtgcaacaacaaactatcgcatcagaatcattagaacaacgtattacgagtcttgagaatggtctaaagccagtttatgatatggcaaaaacaatctcctcattgaacagggtttgtgctgagatggttgcaaaatatgatcttctggtgatgacaaccggtcgggcaacagcaaccactgcggcaactgaggcttattgggctgaacatggtcaaccaccacctggaccatcactttatgaagaaagtgcaattcggggtaagattgaatctagagatgagaccgtccctcaaagtgttagggaggcattcaacaatctagacagtaccacttcactaactgaggaaaattttgggaaacctgacatttcagcaaaggatttgagaaacattatgtatgatcacttgcctggttttggaactgctttccaccaattagtacaagtgatttgtaaattgggaaaagatagcaactcattggatatcattcatgctgagttccaggccagcctggctgaaggagactctcctcaatgtgccctaattcaaattacaaaaagagttccaatcttccaagatgctgctccacctgtcatccacatccgctctcgaggtgacattccccgagcttgccagaaaagcttgcgtccagtcccgccatcacccaagattgatcgaggttgggtatgtgttttccagcttcaagatggtaaaacacttggactcaaaatttgagccaatctcccttccctccgaaagaggcgaccaatagcagaggcttcaactgctgaactacagggtacgttacattaatgatacacttgtgagtatcagccctagataatataagtcaattaaacgaccaagccaaaattgttcatatcccgctagcagcttaaaatataaatgaaataggagctatatctctgacagtattataatcaattgttattaagtaacccaaaccaaaaatgatgaagattaagaaaaacctacctcgactgagagagtgtttttccattaaccttcatcttgtaaacgttgagcaaaattgttacgaatatgaggcgggttatattgcctactgctcctcctgaatatatggaggccatataccctgtcaggtcaaattcaacaattgctaggggtggcaacaacaatacaggcttcctgacaccggagtcagtcaatggagacactccatcgaatccactcaggccaattgctgatgacaccatcgaccatgctagccacacaccaggcagtgtgtcatcagcattcatccttgaagctatggtgaatgtcatatcgggccccaaagtgctaatgaagcaaattccaatttggcttcctctaggtgtcgctgatcaaaagacctacagctttgactcaactacggccgccatcatgcttgcttcatatactatcacccatttcggcaaggcaaccaatccacttgtcagagtcaatcggctgggtcctggaatcccggatcaccccctcaggctcctgcgaattggaaaccaggccttcctccaggagttcgttcttccgccagtccaactaccccagtatttcacctttgatttgacagcactcaaactgatcacccaaccactgcctgctgcaacatggaccgatgacactccaacaggatcaaatggagcgctgcgtccaggaatttcgtttcatccaaaacttcgccccattcttttacctaacaaaagtgggaagaaggggaacagtgccgatctaacatctccagagaaaatccaagcaataatgacttcactccaggactttaagatcgttccaattgatccaaccaaaaatatcatgggtatcgaagtgccagaaactctggtccacaagctgaccggtaagaaggtgacttctaaaaatggacaaccaatcatccctgttcttttgccaaagtacattgggttggacccggtggctccaggagacctcaccatggtaatcacacaggattgtgacacgtgtcattctcctgcaagtcttccagctgtgattgagaagtaattgcaataattgactcagatccagttttacagaatcttctcagggatagtgataacatctatttagtaatccgtctattagaggagatacttttaattgatcaatatactaaaggtgctttacaccattgtcttttttctctcctaaatgtagaacttaacaaaagactcacaatatacttgtcttaaagagattgattgatgaaagatcatgactaataacattacaaataatcctactataatcaatacggtgattcaaatattaatctttctaattgcacatactctctgcccctatcctcaaattgcctacatgcctacatctgaggatagccagtgtgacttggattggagatgtagggaagaaatcggaacccatctccaggttgttcacaatccaagcacagacatcgcccttctaattaagaaaaaatcggcgatgaagattaagccgacagtgagcgcaatcttcatctctcttagattatttgttttccagagtaggggtcatcaggtcctttccaatcatataaccaaaataaacttcactagaaggatattgtgaggcaacaacacaatgggtattacaggaatattgcagttacctcgtgatcgattcaagaggacatcattctttctttgggtaattatccttttccaaagaacattttccatcccacttggagtcatccacaatagcacattacaagttagtgatgtcgacaaactagtttgtcgtgacaaactgtcatccacaaatcaattgagatcagttggactgaatctcgaagggaatggagtggcaactgacgtgccatctgcaactaaaagatggggcttcaggtccggtgtccctccaaaggtggtcaattatgaagctggtgaatgggctgaaaactgctacaatcttgaaatcaaaaaacctgacgggagtgagtgtctaccagcagcgccagacgggattcggggcttcccccggtgccggtatgtgcacaaagtatcaggaacgggaccgtgtgccggagactttgccttccacaaagagggtgctttcttcctgtatgatcgacttgcttccacagttatctaccgaggaacgactttcgctgaaggtgtcgttgcatttctgatactgccccaagctaagaaggacttcttcagctcacaccccttgagagagccggtcaatgcaacggaggacccgtccagtggctactattctaccacaattagatatcaggctaccggttttggaaccaatgagacggagtacttgttcgaggttgacaatttgacctacgtccaacttgaatcaagattcacgccacagtttttgctccagctgaatgagacaatatatgcaagtgggaaaaggagcaacaccacgggaaaactaatttggaaggtcaaccccgaaattgatacaacaatcggggagtgggccttctgggaaactaaaaaaacctcactagaaaaattcgcagtgaagagttgtctttcacagctgtatcaaacggagccaaagacatcagtggtcagagtccggcgcgaacttcttccgacccagagacctacacaacaactgaagaccacaaaatcatggcttcagaaaattcctctgcaatggttcaagtgcacaatcaaggaagggaagctgcagtgtcgcatctgataacccttgccacaatctccacgagtcctcaatcccctacaaccaaaccaggtcaggacaacagcacccataatacacccgtgtataaacttgacatctctgaggcaactcaagttgaacaacatcatcgcagaacagacaacgacagcacagcctccgacactccccccgccacgaccgcagccggacccccaaaagcagagaacatcaacacgagcaagagcgctgactccctggaccccgccaccacgacaagtccccaaaactacagcgagaccgctggcaacaacaacactcatcaccaagataccggagaagagagtgccggcagcgggaagctgggcttgattgccaatactattgctggagtcgcagggctgatcacaggcgggagaagaactcgaagagaagcaattgtcaatgctcaacccaaatgcaaccccaatctacattactggactactcaggatgaaggtgctgcaatcggattggcctggataccatatttcgggccagcagccgagggaatttacacagaggggctaatgcacaatcaagatggtttaatctgtggattgaggcagctggccaatgagacgactcaagctcttcaactgttcctgagagccacaactgagctacgcaccttttcaatcctcaaccgtaaggcaattgatttcttgctgcagcgatggggcggcacatgccacattttgggaccggactgctgtatcgaaccacatgattggaccaagaacataacagacaaaattgatcagattattcatgattttgttgataaaacccttccggaccagggggacaatgacaattggtggactggatggagacaatggataccggcaggtattggagttacaggcgttataattgcagttattgctttattctgtatatgcaaatttgtcttttagtttttcttcagattgcttcatggcaaagctcagcctcaaatcaatgagattaggatttaattatatggatcacttgaatctaagattacttgacaaatgataatataatacactggagctttaaatatagccaatgtgattctaactcctttaaactcacaattaatcataaacaaggtttgacatcaatctagttatatctttgagaatgataaacttgatgaagattaagaaaaaggtaatctttcgattatctttagtcttcatccttgattctacaatcatgacagttgtctttagtgacaagggaaagaagcctttttagtaagttgtaataatcagatctgcgaaccggtagagtttaattgcaacctaacacacataaagcattggtcaaaaagtcaatagaaatttaaacagtgagtggagacaactttcaaatggaagctccatacgagagaggacgcccccgagctgccagacagcattcaagggatggacacgaccatcatgttcgagcacgatcatcatccagagagaattatcgaggtgagtaccgtcaatcaaggagcgcctcacaagtgcgcgttcctactgtatttcataagaggagagttgaaccattaacagttcctccagcacctaaagacatatgtccgaccttgaaaaaaggatttttgtgtgacagtagtttttgcaaaaaagatcaccagttggaaagtttaactgatagggaattactcctactaatcgcccgtaagacttgtggatcagtagaacaacaattaaatataactgcacccaaggactcgcgcttagcaaatccaacggctgatgatttccagcaagaggaaggtccaaaaattaccttgttgacactgatcaagacggcagaacactgggcgagacaagacatcaggaccacagaggattcaaaattaagagcattgttgactctatgtgctgtgatgacgaggaaattctcaaaatcccagctgagtcttttatgtgagacacacctgaggcgcgaggggcttgggcaagatcaggcagaacccgttctcgaagtatatcaacgattacacagtgataaaggaggcagtttcgaagctgcactatggcaacaatgggatcgacaatccctaattatgtttatcactgcattcttgaatatcgctctccagttaccgtgtgaaagttctgctgtcgttgtttcagggttaagaacattggttcctcaatcagataatgaggaagcttcaaccaacccggggacatgctcatggtctgatgatggtaccccttaataaggctgactaaaacactatataaccttctacttgatcacaatactccgtatacctatcatcatatattcaatcaagacggtatcctttaaaacttattcagtactataatcactctcgtttcaaattaataagatatgcataattgctttaatatatgaagaggtatgatacaaccctaacagtgatcaaagaaaatcataatctcttatcgctcgtaatataacctgccaagcatacctcttgcacaaagtgattcttgtacacaaataatgttttactctacaggaggtagcaacgatccatcccatcaaaaaataagtattttatgacttactaatgatctcttaaaatattaagaaaaactgacggaacacaaattctttctgcttcaagttgtggaggaggtctttggtattggctattgttatattacaatcaataacaagcttgtaaaaatattgttcttgtttcaagaggtagattgtgaccggaaacgctaaactaatgatgaagattaatgcggaggtctgataagaataaaccttattattcagattaggccccaagaggcattcttcatctccttttagcaaagtactatttcagggtagtccaattagtgacacgtcttttagctgtatatcagtcgcccctgagatacgccacaaaagtgtctctaagctaaattggtctgtacacatctcatacattgtattaggggcaataatatctaattgaacttagccgtttaaaatttagtgcataaacctgggctaactccaccaggtcaactccattggctgaaaagaagcccacctacaacgaacatcactttgagcgcccttacaattaaaaaataggaacgtcgttccaacaattgagcgcaaggtttcaaggttgaactgagagtgcctaaacaccaaaatatcgataattcagacaccaagcaagacctgagaaggaaccatggctaaagctacgggacgatacaatctaatatcgcccaaaaaggacctggagaaaggggttgtcttaagcgacctctgtaacttcctagttagtcaaactattcaagggtggaaggtctattgggctggtattgagtttgatgtgactcacaaaggaatggccctattgcatagactgaaaactaatgactttgcccctgcatggtcaatgacaaggaatctatttcctcatttatttcaaaatccgaattccacaattgagtcaccactgtgggcattgagagtcatccttgcagcaggggtacaggaccagctgattgaccagtctttgattgaacccttagcaggagcccttggtctgatctctgattggctgctaacaaccaacactaaccatttcaacatgcgaacacaacgtgttaaggaacaattgagcctaaaaatgctgtcgttgattcgatccaatattctcaagtttattaaccaattggatgctctacatgtcgtgaactacaacgggttgttgagcagtattgaaattggaactcaaaatcatacaatcattataactcgaactaacatgggttttctggtggagctccaagaacccgacaaatcggcaatgaaccgcaagaagcctgggccggcgaaattttccctccttcatgagtccacactgaaagcatttacacaagggtcctcgacacgaatgcaaagtttgattcttgaatttaatagctctcttgctatctaattaagatggaatacttcatattgagctaactcatatatgctgactcaatagttatcttgacatctctgctttcataatcagatatataagcataataaataaatactcatatttcttgataatttgtttaaccacagataaatcctaactgtaagccagcttccaagttgacacccttacaaaaaccaggactcagaatccctcaaataagagattccaagacaacatcatagaattgctttattatatgaataagcatgttatcaccagaaatccaatatactaaatagttaattgtaactgaacccgcaggtcacgtgtgttaggtttcacagattatatatattactaactccatacccgtaattaacattagataagtagattaagaaaaacgcttgaggaagattaagaaaaactgcttattgggtctttccgtgttttagatgaagcagttgacattcttcctcttgatattaaatggctacacaacatacccaatacccagacgccaggttatcatcaccaattgtattggaccaatgtgacctagtcactagagcttgcgggttatattcatcatactcccttaatccgcaactacgcaactgtaaactcccgaaacatatctaccgtttaaaatatgatgtaactgttaccaagttcttaagtgatgtaccagtggcgacattgccaatagatttcatagtcccaattcttctcaaggcactgtcaggcaatgggttctgtcctgttgagccgcggtgtcaacagttcttagatgaaatcattaagtacacaatgcaagatgctctcttcctgaaatattatctcaaaaatgtgggtgctcaagaggactgtgttgatgaccactttcaagagaaaatcttatcttcaattcagggcaatgaatttttacatcaaatgttcttctggtatgacctggctattttgactcgaaggggtagattaaatcgaggaaactctagatcaacatggtttgttcatgatgatttaatagacatcttaggctatggggactatgttttttggaagatcccaatttcaatgttacccctgaacacacaaggaatcccccatgctgctatggattggtatcaggcatcagtattcaaagaagcggttcaagggcatacacacattgtttctgtttctactgccgacgtcttgataatgtgcaaagatttaattacatgtcgattcaacacaactctaatctcaaagatagcagaggttgaggatccagtttgttctgattatcccgattttaagattgtgtctatgctttaccagagcggagattacttactctccatattagggtctgatgggtataaaattattaagttcctcgaaccattgtgcttggccaaaattcaattatgctcaaagtacaccgagaggaagggccgattcttaacacaaatgcatttagctgtaaatcacaccctggaagaaattacagaaatgcgtgcactaaagccttcacaggatcaaaagatccgtgaattccatagaacattgataaggctggagatgacgccacaacaactttgtgagctattttccattcaaaaacactgggggcatcctgtgctacatagtgaaacagcaatccaaaaagttaaaaaacatgccacggtgctaaaagcattacgccctatagtgattttcgagacatattgtgtttttaaatatagtattgcaaaacattattttgatagtcaaggatcttggtacagtgttacttcagataggaatttaacgccaggtcttaattcttatatcaaaagaaatcaattccccccgttgccaatgattaaagaactactatgggaattttaccaccttgaccatcctccacttttctcaaccaaaattattagtgacttaagtatttttataaaagacagagctaccgcagtggaaaggacatgctgggatgcagtattcgagcctaatgttctaggatataatccacctcacaaattcagtactaaacgtgtaccagaacaatttttagagcaagaaaacttttctattgagaatgttctttcctacgcgcaaaaactcgagtatctactaccacaataccggaatttttctttctcattgaaagagaaagagttgaatgtaggtagaactttcggaaaattgccttatccgactcgcaatgttcaaacactttgtgaagctctgttagctgatggtcttgctaaagcatttcctagcaatatgatggtagtcacagagcgtgagcaaaaagaaagcttattgcatcaagcatcatggcaccacacaagtgatgattttggtgagcatgccacagttagagggagtagctttgtaactgatttagagaaatacaatcttgcatttagatatgagtttacagcaccttttatagaatattgtaaccgttgctatggtgttaagaatgtttttaattggatgcattatacaatcccccagtgttatatgcatgtcagtgattattataatccaccgcataacctcactctggaaaatcgagacaacccccccgaagggcccagttcatacagaggtcatatgggagggattgaaggactgcaacaaaaactctggacaagtatttcatgtgctcaaatttctttagttgaaataaagactggttttaagttacgctcagctgtgatgggtgacaatcagtgcattaccgttttatcagtcttccccttagagactgacgcagacgagcaggaacagagcgccgaagacaatgcagcgagggtggccgccagcctagcaaaagttacaagtgcctgtggaatctttttaaaacctgatgaaacatttgtacattcaggttttatctattttggaaaaaaacaatatttgaatggggtccaattgcctcagtcccttaaaacggctacaagaatggcaccattgtctgatgcaatttttgatgatcttcaagggaccctggctagtataggcactgcttttgaacgatccatctctgagacacgacatatctttccttgcaggataaccgcagctttccatacgtttttttcggtgagaatcttgcaacatcatcacctcgggttcaataagggttttgaccttggacagttgacacttggcaaacctctggatttcggaacaatatcattggcactagcggtaccgcaggtgcttggagggttatccttcttgaatcctgagaaatgtttctaccggaatttaggagatccagttacctcaggcttattccagttaaaaacttatctccgaatgattgagatggatgatttattcttacctttaattgcgaagaaccctgggaactgcactgccattgactttgtgctaaatcctagcggattaaatgtccccgggtcgcaagacttaacttcatttctgcgccagattgtgcgtaggactatcaccctaagtgcgaaaaacaaacttattaatactttatttcatgcgtcagctgacttcgaagacgaaatggtttgtaaatggctattatcatcaactcctgttatgagtcgttttgcggccgatatcttttcacgcacgcccagtgggaagcgattgcaaattctaggatacctggaaggaacacgcacattattagcctctaagatcatcaacaataatacagaaacaccggttttggacagactgaggaaaataacattgcaaaggtggagtctatggtttagttatcttgatcattgtgataatatcctggcagaggctttaacccaaataacttgcacagttgatttagcacagatcctgagggaatattcatgggcacatattttagaggggagacctcttattggagccacacttccatgtatgattgagcaattcaaagtggtttggctgaaaccctacgaacaatgtccgcagtgttcaaatgcaaagcaacctggtgggaaaccattcgtgtcagtggcagtcaagaaacatattgttagtgcatggccgaacgcatcccgaataagctggactatcggggatggaatcccatacattggatcaaggacagaagataagataggacaacctgctattaaaccaaaatgtccttccgcagccttaagagaggccattgaactggcgtcccgtttaacatgggtaactcaaggcagttcgaacagtgatttgctaataaaaccatttttggaagcacgagtaaatttaagtgttcaagaaatacttcaaatgaccccttcacattactcaggaaatattgttcacaggtacaacgatcaatatagtcctcattctttcatggccaatcgtatgagtaattcagcgacgcgattgattgtttctactaacactttaggtgagttttcaggaggtggccagtctgcacgcgacagcaatattattttccagaatgttataaattatgcagttgcactgttcgatattaaatttagaaacactgaggctacagatatccaatataatcgtgctcaccttcatctaactaagtgttgcacccgggaagtaccagctcagtatttaacatacacatctacattggatttagatttaacaagataccgagaaaacgaattgatttatgacaataatcctctaaaaggaggactcaattgcaatatctcattcgataacccatttttccaaggtaaacggctaaacattatagaagatgatcttattcgactgcctcacttatctggatgggagctagccaagaccatcatgcaatcaattatttcagatagcaacaattcgtctacagacccaattagcagtggagaaacaagatcattcactacccatttcttaacttatcccaagataggacttctgtacagttttggggcctttataagttattatcttggcaatacaattcttcggactaagaaattaacacttgacaattttttatattacttaactacccaaattcataatctaccacatcgctcattgcgaatacttaagccaacattcaaacatgcaagcgttatgtcacggttaatgagtattgatcctcatttttctatttacataggcggtgcggcaggtgacagaggactctcagatgcggccaggttatttttgagaacgtccatttcatcttttcttgcatttataaaagagtggataattaatcgcggaacaattgtccctttatggatagtatatccgctagagggtcaaaacccaacacctgttaataatttcctccatcagatcgtagaactgctggtgcatgattcatcaagacaacaggcttttaaaactaccataagtgatcatgtacatcctcacgacaatcttgtttacacatgtaagagtacagccagcaatttcttccatgcgtcattggcgtactggagaagcaggcacagaaacagcaatcgaaaatacttggcaagagactcttcaactggatcaagcacaaacaacagtgatggtcatattgagagaagtcaagaacaaaccaccagagatccacatgatggcactgaacggaatctagtcctacaaatgagccatgaaataaaaagaacgacaattccacaagaaagcacgcaccagggtccgtcgttccagtcatttctaagtgactctgcttgtggtacagcaaatccaaaactaaatttcgatagatcgagacataatgtgaaatctcaggatcataactcggcatccaagagggaaggtcatcaaataatctcacaccgtctagtcctacctttctttacattgtctcaagggacgcgccaattaacgtcatccaatgagtcacaaacccaagacgagatatcaaagtacttacggcaattgagatccgtcattgataccacagtttattgtaggtttaccggtatagtctcgtccatgcattacaaacttgatgaggtcctttgggaaatagagagttttaagtcggctgtgacgctagcagagggagaaggtgctggtgccttactattgattcagaaataccaagttaagaccttatttttcaacacgctagctactgagtccagtatagagtcagaaatagtatcaggaacgactactcctaggatgcttctacctgttatgtcaaaattccataatgaccaaattgagattattcttaacaattcggcaagccaaataacagacataacaaatcctacttggttcaaagaccaaagagcaaggctacctaggcaagtcgaggttataaccatggatgcagagacgacagaaaatataaacagatcgaaattgtacgaagctgtatataaattgatcttacaccatattgatcccagcgtattgaaagcagtggtccttaaagtctttctaagtgatactgagggtatgttatggctaaatgataatttagccccgttttttgccactggttatttaattaagccaataacgtcaagtgctagatctagtgagtggtatctttgtctgacgaacttcttatcaactacacgtaagatgccacaccaaaaccatctcagttgtaaacaggtaatacttacggcattgcaactgcaaattcaacggagcccatactggctaagtcatttaactcagtatgctgactgcgatttacatttaagttatatccgccttggttttccatcattagagaaagtactataccacaggtataacctcgtcgattcaaaaagaggtccactagtctctatcactcagcacttggcacatcttagagcagagattcgagaattgactaatgattataatcaacagcgacaaagtcggactcaaacatatcactttattcgtactgcaaaaggacgaatcacaaaactagtcaatgattatttaaaattctttcttattgtgcaagcattaaaacataatgggacatggcaagctgagtttaagaaattaccagagttgattagtgtgtgcaataggttctatcatattagagattgcaattgtgaagaacgtttcttagttcaaaccttatatctacatagaatgcaggattctgaagttaagcttatcgaaaggctgacagggcttctgagtttattcccggatggtctctacaggtttgattgaattaccgtgcatagtatcctgatacttgtgaaggttgattatcaacgtacagattataaaaaactcacaaattgctctcatacatcatattgatcgaatttcaataaataactatttaaataacgaaagaagtccttatattatacactatatttagcctctctccctgcgtgataatcaaaaaattcacaatgcagcatgtgtgacatattacttccgcgatgaatctaacgcaacataataaactctgcactctttataattaagctttaacaaaaggtctgggctcatattgttattgatataataatgttgtatcaatatcctgtcagatggaatagtgttttggttgataacacgacttcttaaaacaaaattgatcttcaagattaagttttttataattatcattactttaatttgtcgatttaaaaatggtgatagccttaatctttgtgtaaaataagagattaggtgtaataactttaacattttgtctagtaagctactatttcatacagaatgataaaattaaaagaaaaggcatgactgtaaaatcagaaataccttctttacaatatagcagactagataataatcttcgtgttaatgataattaagacattgaccacgctcatcaggaggctcgccaggataaacgttgcaaaaaggattcctggaaaaatggtcgcacacaaaaatttaaaaataaatctatttcttcttttttgtgtgtcca

SEQ ID NO: 27 - DNA, 341 bp, Homo sapiens
  source 1..341: mol_type = other DNA
atagggcggagggaagctcatcagtggggccacgagctgagtgcgtcctgtcactccactcccatgtcccttgggaaggtctgagactagggccagaggcggccctaacagggctctccctgagcttcggggaggtgagttcccagagaacggggctccgcgcgaggtcagactgggcaggagatgccgtggaccccgcccttcggggaggggcccggcggatgcctcctttgccggagcttggaacagactcacggccagcgaagtgagttcaatggctgaggtgaggtaccccgcaggggacctcataacccaattcagactactctcctccgcccatt

SEQ ID NO: 28-30 - skipped (3 entries)

SEQ ID NO: 31 - DNA, 12 bp, synthetic construct
  misc_feature 1..12: note = Description of Artificial Sequence: Synthetic oligonucleotide
  variation 6..7: note = a, c, t, g, unknown or other
  source 1..12: mol_type = other DNA
gactcnngagtc

SEQ ID NO: 32 - DNA, 14 bp, synthetic construct
  misc_feature 1..14: note = Description of Artificial Sequence: Synthetic oligonucleotide
  variation 6..9: note = a, c, t, g, unknown or other
  source 1..14: mol_type = other DNA
gactcnnnngagtc

SEQ ID NO: 33 - DNA, 16 bp, synthetic construct
  misc_feature 1..16: note = Description of Artificial Sequence: Synthetic oligonucleotide
  variation 6..11: note = a, c, t, g, unknown or other
  source 1..16: mol_type = other DNA
gactcnnnnnngagtc

SEQ ID NO: 34 - DNA, 24 bp, synthetic construct
  misc_feature 1..24: note = Description of Artificial Sequence: Synthetic oligonucleotide
  variation 1..4: note = a, c, t, g, unknown or other
  variation 10..15: note = a, c, t, g, unknown or other
  variation 21..24: note = a, c, t, g, unknown or other
  source 1..24: mol_type = other DNA
nnnngactcnnnnnngagtcnnnn

SEQ ID NO: 35 - DNA, 22 bp, synthetic construct
  misc_feature 1..22: note = Description of Artificial Sequence: Synthetic oligonucleotide
  variation 1..4: note = a, c, t, g, unknown or other
  variation 10..13: note = a, c, t, g, unknown or other
  variation 19..22: note = a, c, t, g, unknown or other
  source 1..22: mol_type = other DNA
nnnngactcnnnngagtcnnnn

SEQ ID NO: 36 - DNA, 23 bp, synthetic construct
  misc_feature 1..23: note = Description of Artificial Sequence: Synthetic primer
  source 1..23: mol_type = other DNA
cttcttagcttggggcagtatca

SEQ ID NO: 37 - DNA, 500 bp, synthetic construct
  misc_feature 1..500: note = Description of Artificial Sequence: Synthetic polynucleotide
  source 1..500: mol_type = other DNA
aagatgactgcaggagtcaatgcgcagttggtcccggcagaccaggcgaacattaccgaattttacaacaagtccctttcatcctacaaggagaatgaggagaacatccagtgtggggagaacttcatggacatggagtgcttcatgattctgaaccccagtcagcagctggcaattgccgtcttgtctctcacactgggcaccttcacagttctggagaacttgctggtgctgtgtgtcaccacagttatctaccgaggaacgactttcgctgaaggtgtcgttgcatttctgattccttcactcccgcagcctccgctgccggccctcttaccacttcatcattagcctggccgtggccgaccttctggggagtgtcatttttgtctacagctttgttgactttcatgtgttccaccgcaaggacagccccaacgtctttctcttcaaattgggtggggtcaccgcctccttcacggcctctgtaggcagcctcttcc

SEQ ID NO: 38 - RNA, 89 nt, synthetic construct
  misc_feature 1..89: note = Description of Artificial Sequence: Synthetic oligonucleotide
  source 1..89: mol_type = other RNA
gactgcaggagtctgctgcttccacagttatctaccgaggaacgactttcgctgaaggtgtcgttgcatttctgattccttcactcccg

SEQ ID NO: 39 - DNA, 36 bp, synthetic construct
  misc_feature 1..36: note = Description of Artificial Sequence: Synthetic primer
  modified_base 24: mod_base = OTHER; note = Methoxy base
  modified_base 31..36: mod_base = OTHER; note = Methoxy base
  source 1..36: mol_type = other DNA
gactcgatatcgagtccacgagctgagtgcgtcctg

SEQ ID NO: 40 - DNA, 35 bp, synthetic construct
  misc_feature 1..35: note = Description of Artificial Sequence: Synthetic primer
  modified_base 24: mod_base = OTHER; note = Methoxy base
  modified_base 31..35: mod_base = OTHER; note = Methoxy base
  source 1..35: mol_type = other DNA
gactcgatatcgagtcagaccttcccaagggacat

SEQ ID NO: 41 - DNA, 27 bp, synthetic construct
  misc_feature 1..27: note = Description of Artificial Sequence: Synthetic probe
  source 1..27: mol_type = other DNA
ccacgcctgtcactccactccgcgtgg

SEQ ID NO: 42 - DNA, 19 bp, synthetic construct
  misc_feature 1..19: note = Description of Artificial Sequence: Synthetic primer
  source 1..19: mol_type = other DNA
cctctggccctagtctcag

SEQ ID NO: 43 - DNA, 17 bp, synthetic construct
  misc_feature 1..17: note = Description of Artificial Sequence: Synthetic primer
  source 1..17: mol_type = other DNA
gcatctaatttttcgcc

SEQ ID NO: 44 - DNA, 17 bp, synthetic construct
  misc_feature 1..17: note = Description of Artificial Sequence: Synthetic primer
  source 1..17: mol_type = other DNA
tctgtgcctggattgat

SEQ ID NO: 45 - DNA, 17 bp, synthetic construct
  misc_feature 1..17: note = Description of Artificial Sequence: Synthetic primer
  source 1..17: mol_type = other DNA
ttttggacgtcttctcc

SEQ ID NO: 46 - DNA, 17 bp, synthetic construct
  misc_feature 1..17: note = Description of Artificial Sequence: Synthetic primer
  source 1..17: mol_type = other DNA
ttttgaacgtcttctcc

SEQ ID NO: 47 - DNA, 25 bp, synthetic construct
  misc_feature 1..25: note = Description of Artificial Sequence: Synthetic primer
  source 1..25: mol_type = other DNA
tatgttttgtataaaagttcatttg

SEQ ID NO: 48 - DNA, 16 bp, Zaire ebolavirus
  source 1..16: mol_type = other DNA
acgactttygctgaag

SEQ ID NO: 49 - DNA, 16 bp, Zaire ebolavirus
  source 1..16: mol_type = other DNA
acgactttcgctgaag

SEQ ID NO: 50 - DNA, 56 bp, Zaire ebolavirus
  source 1..56: mol_type = other DNA
tccacagttatctaccgaggaacgactttcgctgaaggtgtcgttgcatttctgat

SEQ ID NO: 51 - DNA, 59 bp, Zaire ebolavirus
  source 1..59: mol_type = other DNA
ttccacagttatctaccgaggaacgactttcgctgaaggtgtcgttgcatttctgatac

SEQ ID NO: 52 - DNA, 26 bp, synthetic construct
  misc_feature 1..26: note = Description of Artificial Sequence: Synthetic probe
  modified_base 14: mod_base = OTHER; note = Methoxy base
  source 1..26: mol_type = other DNA
gctacacgactttggctgaaggtagc

SEQ ID NO: 53 - DNA, 25 bp, synthetic construct
  misc_feature 1..25: note = Description of Artificial Sequence: Synthetic probe
  source 1..25: mol_type = other DNA
gctaccttcagcraaagtcggtagc

SEQ ID NO: 54 - DNA, 36 bp, synthetic construct
  misc_feature 1..36: note = Description of Artificial Sequence: Synthetic primer
  modified_base 24: mod_base = OTHER; note = Methoxy base
  modified_base 32..36: mod_base = OTHER; note = Methoxy base
  source 1..36: mol_type = other DNA
gactcgatatcgagtccttccacagttatctaccga

SEQ ID NO: 55 - DNA, 36 bp, synthetic construct; identical to SEQ ID NO: 1
  misc_feature 1..36: note = Description of Artificial Sequence: Synthetic primer
  modified_base 24: mod_base = OTHER; note = Methoxy base
  modified_base 32..36: mod_base = OTHER; note = Methoxy base
  source 1..36: mol_type = other DNA

SEQ ID NO: 56 - DNA, 36 bp, synthetic construct
  misc_feature 1..36: note = Description of Artificial Sequence: Synthetic primer
  modified_base 24: mod_base = OTHER; note = Methoxy base
  modified_base 32..36: mod_base = OTHER; note = Methoxy base
  source 1..36: mol_type = other DNA
gactcgatatcgagtcacagttatctaccgaggaac

SEQ ID NO: 57 - DNA, 33 bp, synthetic construct
  misc_feature 1..33: note = Description of Artificial Sequence: Synthetic primer
  modified_base 24: mod_base = OTHER; note = Methoxy base
  modified_base 29..33: mod_base = OTHER; note = Methoxy base
  source 1..33: mol_type = other DNA
gactcgatatcgagtcaaatgcaacgacacctt

SEQ ID NO: 58 - DNA, 33 bp, synthetic construct; identical to SEQ ID NO: 2
  misc_feature 1..33: note = Description of Artificial Sequence: Synthetic primer
  modified_base 24: mod_base = OTHER; note = Methoxy base
  modified_base 29..33: mod_base = OTHER; note = Methoxy base
  source 1..33: mol_type = other DNA

SEQ ID NO: 59 - DNA, 33 bp, synthetic construct
  misc_feature 1..33: note = Description of Artificial Sequence: Synthetic primer
  modified_base 24: mod_base = OTHER; note = Methoxy base
  modified_base 29..33: mod_base = OTHER; note = Methoxy base
  source 1..33: mol_type = other DNA
gactcgatatcgagtcagaaatgcaacgacacc

SEQ ID NO: 60 - DNA, 36 bp, synthetic construct
  misc_feature 1..36: note = Description of Artificial Sequence: Synthetic primer
  modified_base 24: mod_base = OTHER; note = Methoxy base
  modified_base 32..36: mod_base = OTHER; note = Methoxy base
  source 1..36: mol_type = other DNA
gactcgcgcgcgagtcacagttatctaccgaggaac

SEQ ID NO: 61 - DNA, 36 bp, synthetic construct
  misc_feature 1..36: note = Description of Artificial Sequence: Synthetic primer
  modified_base 24: mod_base = OTHER; note = Methoxy base
  modified_base 32..36: mod_base = OTHER; note = Methoxy base
  source 1..36: mol_type = other DNA
gactcgcgcgcgagtccacagttatctaccgaggaa

SEQ ID NO: 62 - DNA, 36 bp, synthetic construct
  misc_feature 1..36: note = Description of Artificial Sequence: Synthetic primer
  modified_base 24: mod_base = OTHER; note = Methoxy base
  modified_base 32..36: mod_base = OTHER; note = Methoxy base
  source 1..36: mol_type = other DNA
gactcgcgcgcgagtcccacagttatctaccgagga

SEQ ID NO: 63 - DNA, 33 bp, synthetic construct
  misc_feature 1..33: note = Description of Artificial Sequence: Synthetic primer
  modified_base 24: mod_base = OTHER; note = Methoxy base
  modified_base 29..33: mod_base = OTHER; note = Methoxy base
  source 1..33: mol_type = other DNA
gactcgcgcgcgagtcaaatgcaacgacacctt

SEQ ID NO: 64 - DNA, 33 bp, synthetic construct
  misc_feature 1..33: note = Description of Artificial Sequence: Synthetic primer
  modified_base 24: mod_base = OTHER; note = Methoxy base
  modified_base 29..33: mod_base = OTHER; note = Methoxy base
  source 1..33: mol_type = other DNA
gactcgcgcgcgagtcgaaatgcaacgacacct

SEQ ID NO: 65 - DNA, 33 bp, synthetic construct
  misc_feature 1..33: note = Description of Artificial Sequence: Synthetic primer
  modified_base 24: mod_base = OTHER; note = Methoxy base
  modified_base 29..33: mod_base = OTHER; note = Methoxy base
  source 1..33: mol_type = other DNA
gactcgcgcgcgagtcagaaatgcaacgacacc

SEQ ID NO: 66 - DNA, 55 bp, synthetic construct
  misc_feature 1..55: note = Description of Artificial Sequence: Synthetic primer
  source 1..55: mol_type = other DNA
ccacagttatctaccgaggaacgactttcgctgaaggtgtcgttgcatttctgat

SEQ ID NO: 67 - DNA, 71 bp, synthetic construct
  misc_feature 1..71: note = Description of Artificial Sequence: Synthetic oligonucleotide
  source 1..71: mol_type = other DNA
tgactagcggaggctagaaggagagagatgggtgcgagagcgtcaatattaagaggcgaaaaattagatgc

SEQ ID NO: 68 - DNA, 48 bp, synthetic construct
  misc_feature 1..48: note = Description of Artificial Sequence: Synthetic oligonucleotide
  source 1..48: mol_type = other DNA
tgactagcagaggctagaaggagagagatgggtgcgagagcgtcagta

SEQ ID NO: 69 - DNA, 71 bp, synthetic construct
  misc_feature 1..71: note = Description of Artificial Sequence: Synthetic oligonucleotide
  source 1..71: mol_type = other DNA
caaaaacagcatattgacgctgggaaagaccagagatcctgctgtctctrcaacatcaatccaggcacaga

SEQ ID NO: 70 - DNA, 47 bp, synthetic construct
  misc_feature 1..47: note = Description of Artificial Sequence: Synthetic oligonucleotide
  source 1..47: mol_type = other DNA
caaaaacagcatattgacgctgggaaagaccacagatcctgctgtct

SEQ ID NO: 71 - DNA, 73 bp, synthetic construct
  misc_feature 1..73: note = Description of Artificial Sequence: Synthetic oligonucleotide
  source 1..73: mol_type = other DNA
aaatgcagatggtctcagctatgaacacagcaaaaacaatgaatggaatgggaaaaggagaagacgtccaaaa

SEQ ID NO: 72 - DNA, 73 bp, synthetic construct
  misc_feature 1..73: note = Description of Artificial Sequence: Synthetic oligonucleotide
  source 1..73: mol_type = other DNA
aaatgcaaatggtctcagctatgaacacagcaaagacaatgaatggaatgggaaagggagaagacgttcaaaa

SEQ ID NO: 73 - DNA, 60 bp, synthetic construct
  misc_feature 1..60: note = Description of Artificial Sequence: Synthetic oligonucleotide
  source 1..60: mol_type = other DNA
aaatgcagatggtttcagctatgaacacagcaaaagcaatgaatggaatggggaaaggag

SEQ ID NO: 74 - DNA, 81 bp, synthetic construct
  misc_feature 1..81: note = Description of Artificial Sequence: Synthetic oligonucleotide
  source 1..81: mol_type = other DNA
ggcccactgtattgctactgaaaatctctgctgtacatggcacatggagttgatcacaaatgaacttttatacaaaacata

SEQ ID NO: 75 - DNA, 58 bp, synthetic construct
  misc_feature 1..58: note = Description of Artificial Sequence: Synthetic oligonucleotide
  source 1..58: mol_type = other DNA
ggcccactgcactgctactaaaaatctctgctgtacatggcacatggagttgatcaca

(19) United States
(12) Patent Application Publication
(10) Pub. No.: US 2026/0092337 A1
(43) Pub. Date: Apr. 2, 2026

(54) COMPOSITIONS AND METHODS FOR DETECTING AN RNA VIRUS

(71) Applicant: ENVIROLOGIX INC., Portland, ME (US)

(72) Inventors: Lars PETERS, Portland, ME (US); Stephen A. JUDICE, Portland, ME (US); Daniel SHAFFER, Portland, ME (US); Breck PARKER, Portland, ME (US)

(73) Assignee: ENVIROLOGIX INC., Portland, ME (US)

(21) Appl. No.: 19/256,489

(22) Filed: Jul. 1, 2025

Related U.S. Application Data
(63) Continuation of application No. 16/991,216, filed on Aug. 12, 2020, now Pat. No. 12,392,001.
Application No. 16/991,216 is a continuation of application No. 15/520,328, now Pat. No. 10,793,922.
(60) Provisional application No. 62/066,277, filed on Oct. 20, 2014.
(60) Provisional application No. 62/104,008, filed on Jan. 15, 2015.

(51) Int. Cl. C12Q 1/70; C12Q 1/6865

(52) U.S. Cl. CPC C12Q 1/701; C12Q 1/6865; C12Q 2600/158; Y02A 50/30

(57) ABSTRACT

The present invention provides methods for rapidly identifying an RNA viral infection using an isothermal nucleic acid amplification reaction that can be carried out on extracted RNA in the context of a crude biological sample.

### CROSS REFERENCE TO RELATED APPLICATIONS

This application is a continuation of U.S. patent application Ser. No. 16/991,216, filed Aug. 12, 2020, now U.S. Pat. No. 12,392,001, which is a continuation of U.S. patent application Ser. No. 15/520,328, filed on Apr. 19, 2017, now U.S. Pat. No. 10,793,922; which is the U.S. national phase application, pursuant to 35 U.S.C. § 371, of PCT International Application Serial No.: PCT/US2015/056491, filed Oct. 20, 2015, designating the United States and published in English, which claims priority to and the benefit of U.S. Provisional Patent Application Serial Nos. 62/066,277, filed Oct. 20, 2014, and 62/104,008, filed Jan. 15, 2015. The entire contents of each of these applications are hereby incorporated by reference herein.

### SEQUENCE LISTING

This application contains a Sequence Listing which has been submitted electronically in XML format and is hereby incorporated by reference in its entirety. The Sequence Listing XML file, created on Dec. 16, 2025, is named 167665-010445USCON2_SL.xml and is 153,605 bytes in size.

### BACKGROUND OF THE INVENTION

The Ebola virus causes hemorrhagic fever with mortality rates reaching 50% to 90% of infected humans. Ebola virus (EBOV) includes four species, Zaire EBOV, Sudan EBOV, Ivory Coast EBOV, and Reston EBOV. Human infection with Ebola typically results from contact with contaminated blood, tissues, and/or excretions of animals or patients with an Ebola infection. Patients typically exhibit symptoms 4 to 10 days after Ebola infection. This long incubation period provides an opportunity for the virus to be carried to new areas before the carrier displays any signs of illness. Symptoms of Ebola include fever, chills, malaise, and myalgia. Because such symptoms are displayed in a variety of illnesses, there is a significant risk that Ebola infection may be misdiagnosed in the early stages, thereby facilitating spread of the disease. In later stages, Ebola-infected subjects typically develop vomiting, diarrhea, coughing, vascular symptoms, headache, confusion, coma, mucosal hemorrhages, bloody diarrhea and ultimately multiorgan failure, resulting in death. The bodily fluids of Ebola patients are highly infectious as are the dead bodies of Ebola patients.

Public health concerns about Ebola infection are mounting as Ebola infections in West Africa in late 2014 are predicted to rise to 10,000 people per week. Because of their exposure to the bodily fluids of Ebola patients, health care workers are at risk for catching Ebola from infected patients. The risk of infection increases as the extent and the frequency of contact increased. In a 1976 Sudan Ebola outbreak 81% of healthcare workers nursing Ebola patients were infected with the virus. In order for medical staff and health care workers to avoid unnecessary infections, early detection of Ebola is critical so that appropriate infection control measures are instituted and the risk of transmission is minimized.

To stop the spread of Ebola within West Africa and internationally, rapid diagnosis is essential so that infected subjects may be immediately quarantined and proper protective equipment used by health care workers caring for these subjects. High titers of infectious filovirus are present in the blood and tissues during early stages of illness. Currently, Ebola is identified by virus isolation, reverse transcription-PCR (RT-PCR), including real-time quantitative RT-PCR, antigen-capture enzyme-linked immunosorbent assay (ELISA), antigen detection by immunostaining, and IgG- and IgM-ELISA using authentic virus antigens. Unfortunately, these tests are time-consuming because they can only be carried out on purified and isolated RNA and require access to laboratory equipment and trained technicians that are scarce in many areas where Ebola is endemic.

Accordingly, improved methods for rapidly identifying patients infected with Ebola virus are urgently required.

### SUMMARY OF THE INVENTION

The present invention provides methods for rapidly identifying an Ebola infection using an isothermal nucleic acid amplification reaction that can be carried out on extracted RNA in the context of a crude biological sample.

In one aspect, the invention provides a method of detecting a specific target polynucleotide (e.g., RNA) in an isothermal amplification reaction coupled with reverse transcription, the method involving
(a) contacting a target polynucleotide molecule in a sample with a primer in the presence of a reverse transcriptase and dNTPs under conditions permissive for cDNA synthesis, thereby generating a cDNA;
(b) contacting the cDNA with forward and reverse primers each carrying at least one nicking enzyme recognition sequence within their respective 5′-terminal regions which specifically bind the cDNA with their respective 3′-terminal regions in the presence of a nicking enzyme, dNTPs, a detectable oligonucleotide probe, and a strand-displacement polymerase under conditions permissive for the isothermal amplification of the cDNA; and
(c) detecting a signal specific for detectable oligonucleotide probe hybridization to the amplicon, where detection of the signal indicates the presence or quantity of the target polynucleotide present in the sample and failure to detect the signal indicates the absence of target polyribonucleotide in the sample.

In another aspect, the invention provides a method of detecting an RNA virus in a sample, the method involving
(a) contacting an RNA virus polynucleotide molecule in a biological sample with a primer in the presence of a reverse transcriptase and dNTPs under conditions permissive for cDNA synthesis, thereby generating a cDNA;
(b) contacting the cDNA with forward and reverse primers each carrying at least one nicking enzyme recognition sequence within their respective 5′-terminal regions which specifically bind the cDNA with their respective 3′-terminal regions in the presence of a nicking enzyme, dNTPs, a detectable oligonucleotide probe, and a strand-displacement polymerase under conditions permissive for the isothermal amplification of the cDNA; and
(c) detecting a signal specific for detectable oligonucleotide probe hybridization to the amplicon, where detection of the signal indicates the presence or quantity of the RNA virus polynucleotide molecule present in the sample and failure to detect the amplicon indicates the absence of an RNA virus.

In a related aspect, the invention provides a method of detecting an Ebola virus in a sample, the method involving
(a) contacting an Ebola polynucleotide molecule in a biological sample with a primer in the presence of a reverse transcriptase and dNTPs under conditions permissive for cDNA synthesis, thereby generating a cDNA;
(b) contacting the cDNA with forward and reverse primers each carrying at least one nicking enzyme recognition sequence within their respective 5′-terminal regions which specifically bind the cDNA with their respective 3′-terminal regions in the presence of a nicking enzyme, dNTPs, a detectable oligonucleotide probe, and a strand-displacement polymerase under conditions permissive for the isothermal amplification of the cDNA; and
(c) detecting a signal specific for detectable oligonucleotide probe hybridization to the amplicon, where detection of the signal indicates the presence or quantity of the Ebola polynucleotide present in the sample and failure to detect the signal indicates the absence of Ebola polynucleotide present in the sample.

In various embodiments of the above aspects or any other aspect of the invention delineated herein, the Ebola polynucleotide is obtained by contacting a biological sample with an agent capable of extracting an RNA molecule present in the sample and an agent capable of stabilizing an RNA molecule against degradation.

In yet another aspect, the invention provides a method of detecting an Ebola virus in a sample, the method involving
(a) contacting a biological sample with an agent capable of extracting a polynucleotide molecule present in the sample and an agent capable of stabilizing a polynucleotide molecule against degradation;
(b) contacting the extracted and stabilized Ebola RNA with a primer in the presence of a reverse transcriptase and dNTPs under conditions permissive for cDNA synthesis, thereby generating an Ebola cDNA;
(c) contacting the Ebola cDNA with forward and reverse primers each carrying at least one nicking enzyme recognition sequence within their respective 5′-terminal regions which specifically bind the Ebola cDNA with their respective 3′-terminal regions in the presence of a nicking enzyme, dNTPs, and a strand-displacement polymerase under conditions permissive for the isothermal amplification of the cDNA, thereby generating amplicons; and
(d) detecting the amplicons, where the presence of an Ebola amplicon detects an Ebola polynucleotide in the sample and failure to detect the amplicon indicates the absence of an Ebola polynucleotide in the sample.

In yet another aspect, the invention provides a kit for detecting an RNA virus polynucleotide molecule involving primers that specifically bind an RNA viral sequence, a detectable probe that specifically binds a viral (e.g., Ebola) amplicon, a reverse transcriptase enzyme, a nicking enzyme, and a strand-displacement polymerase. In one embodiment, the primers contain the following sequences:

### Forward Primer:

GACTCGATATCGAGTCGCTTCCA[MeOC]AGTTATC[MeOU][MeOA][MeOC][MeOC][MeOG](SEQ ID NO: 1)

### Reverse Primer:

GACTCGATATCGAGTCGAAATGC[MeOA]ACGA[MeOC][MeOA][MeOC][MeOC][MeOU](SEQ ID NO: 2); and the probe contains the following sequence: gctacACGACTTTYGCTGAAGgtagc (SEQ ID NO: 3).

In another embodiment, the probe has a fluorescent dye at the 5′ end, and a quencher at the 3′ end or vice versa. In one embodiment, the probe is
5′-CALRed₆₁₀ₙₘ—gctacACGACTTTYGCTGAAGgtagc BHQ2-3′ (SEQ ID NO: 4) or
5′-FAM or FITC—gctacACGACTTTYGCTGAAGgtagc-BHQ1-3′ (SEQ ID NO: 5). In one embodiment, the 3′ quencher is replaced by DABsyl.

In another aspect, the invention provides a kit for amplifying an Ebola polynucleotide molecule in a reverse transcriptase nicking amplification reaction, the kit containing the following primers:

Forward primer:
(SEQ ID NO: 1)
GACTCGATATCGAGTCGCTTCCA[MeOC]AGTTATC[MeOU][MeOA]
[MeOC][MeOC][MeOG]
Reverse Primer:
(SEQ ID NO: 2)
GACTCGATATCGAGTCGAAATGC[MeOA]ACGA[MeOC][MeOA]
[MeOC][MeOC][MeOU];
the following probe:
(SEQ ID NO: 3)
gctacACGACTTTYGCTGAAGgtagc;

a reverse transcriptase enzyme, a nicking enzyme, a strand-displacement polymerase, and directions for use of the aforementioned primers, probes and enzymes for detecting an Ebola polynucleotide molecule.

In one embodiment, the kit further contains a capillary tube that may or may not contain lyophilized lysis or RNA stabilization reagents for viral polynucleotide extraction. In another embodiment, the kit further contains one or more vessels containing a buffer suitable for carrying out a reverse transcriptase and/or amplification reaction. In another embodiment, the kit further contains vessels containing the reverse transcriptase enzyme, nicking enzyme, and strand-displacement polymerase in lyophilized form.

In yet another aspect, the invention provides a method of diagnosing a human or animal subject with an RNA virus, the method involving
(a) contacting a sample of the subject with an agent capable of extracting an RNA virus present in the sample and an agent capable of stabilizing the extracted polynucleotide molecule against degradation;
(b) contacting the polynucleotide molecule with a reverse transcriptase primer in the presence of a reverse transcriptase and dNTPs under conditions permissive for cDNA synthesis, thereby generating a cDNA copy of the polynucleotide molecule;
(c) contacting the cDNA with forward and reverse primers carrying at least one nicking enzyme recognition sequence within their respective 5′-terminal regions which specifically bind the cDNA with their respective 3′-terminal regions in the presence of a nicking enzyme, dNTPs, and a strand-displacement polymerase under conditions permissive for the isothermal amplification of the cDNA, thereby generating amplicons; and
(d) detecting the amplicons, where the presence of an RNA viral amplicon diagnoses an RNA viral infection in the subject and failure to detect the amplicon diagnoses the absence of an RNA viral infection in the subject.

In various embodiments of any aspect delineated herein, no detectable signal is present in a control assay lacking a target polynucleotide at seven minutes, ten minutes, and/or fifteen minutes following initiation of the assay. In other embodiments of any aspect delineated herein, the primer used in step (a) has the same sequence or a different sequence than a primer used in step (b). In other embodiments of any of the above, steps (a)-(c) are carried out in a single reaction. In still other embodiments of the above aspects, the reverse transcriptase enzyme and the strand-displacement DNA polymerase are the same or different enzymes. In still other embodiments, the cDNA of step (a) is generated in a first reaction vessel, then transferred to a second reaction vessel where step (b) is carried out. In still other embodiments of any aspect delineated herein, the polynucleotide molecule is an Ebola polynucleotide. In still other embodiments of any aspect delineated herein, the sample is a bodily fluid (e.g., saliva, sweat, tears, fluids accumulating in a bodily cavity, urine, ejaculate, vaginal secretion, cerebrospinal fluid, lymph, feces, sputum, decomposition fluid, vomit, sweat, breast milk, blood, serum, and plasma). In still other embodiments of any aspect delineated herein, the bodily cavity is peritoneal cavity or pericardial cavity. In still other embodiments of any aspect delineated herein, the limit of detection is 10 or 20 copies per reaction. In still other embodiments of any aspect delineated herein, the method is carried out in about 5, 7, 10, 15, 20, 25 or thirty minutes. In still other embodiments of any aspect delineated herein, steps a-d are carried out in the context of the biological sample. In still other embodiments of any aspect delineated herein, Ebola or other viral RNA is not purified or isolated away from the biological sample (e.g, crude). In still other embodiments of any aspect delineated herein, the method is carried out at a point of care or diagnosis in a portable battery powered device. In still other embodiments of any aspect delineated herein, no separate reverse transcriptase primer is required, but the forward and/or reverse primers are used. In still other embodiments of any aspect delineated herein, the sample is a biological sample or an environmental sample. In still other embodiments of any aspect delineated herein, the biological sample is obtained from a subject, bat, bush meat, or a domestic animal. In still other embodiments of any aspect delineated herein, the biological sample is a swab of a mucosal membrane that is any one or more of buccal, nasal, eye, rectal, and vaginal or skin. In still other embodiments of any aspect delineated herein, the biological sample is a tissue sample obtained from a subject, necropsy, or culture media. In still other embodiments of any aspect delineated herein, the necropsy is of a human, primate, bat, or other mammal. In still other embodiments of any aspect delineated herein, the environmental sample is a material that may be contaminated with a biological fluid of a subject having or having a propensity to develop an Ebola viral infection. In still other embodiments of any aspect delineated herein, the environmental sample is bedding, a seat cushion, a rug, an air condition filter or other material. In still other embodiments of any aspect delineated herein, the polymerase are 5′-exo derivatives of Bst DNA polymerase I, Gst DNA polymerase I, Gka DNA polymerase I, Gca DNA polymerase I, Gan DNA polymerase I, Gbo DNA polymerase I, Gsp70 DNA polymerase I, GspT3 DNA polymerase I, Gsp52 DNA polymerase I and/or fragments thereof. In still other embodiments of any aspect delineated herein, the nicking enzyme is one or more of Nt.BstNBI, Nt.BspD6I, Nt.BspQI, Nt.BsmAI, Nt.AlwI, N.Bst9I, or N.BstSEI. In still other embodiments of any aspect delineated herein, the reverse transcriptase is M-MLV RT, AMV RT, RSV RT, and/or mutants/derivates thereof. In still other embodiments of any aspect delineated herein, the detectable probe contains a molecular beacon. In various embodiments of any aspect delineated herein, an amplification primer (e.g., forward and/or reverse primer) comprises one or more 2′ modified nucleotides (e.g., 2′-O-methyl ribonucleotides) in the 3′ terminal region or recognition region. In particular embodiments, the amplification primer comprises one or more 2′-O-methyl modified nucleotides at the 3′ end, including for example 2′-O-methyl, 2′-methoxyethoxy, 2′-fluoro, 2′-hydroxyl, 2′-alkyl, 2′-allyl, 2′-O-[2-(methylamino)-2-oxoethyl], 4′-thio, 4′-CH₂—O-2′-bridge, 4′-(CH₂)₂—O-2′-bridge, 2′-LNA, and 2′-O—(N-methylcarbamate).

In still other embodiments of any aspect delineated herein, the forward and reverse primers for detection of Ebola virus contain the following sequences, respectively:

Forward primer:
(SEQ ID NO: 1)
GACTCGATATCGAGTCGCTTCCA[MeOC]AGTTATC[MeOU][MeOA]
[MeOC][MeOC][MeOG]
Reverse Primer:
(SEQ ID NO: 2)
GACTCGATATCGAGTCGAAATGC[MeOA]ACGA[MeOC][MeOA]
[MeOC][MeOC][MeOU].

In still other embodiments of any aspect delineated herein, amplification is detected using a probe having the following sequence: gctacACGACTTTYGCTGAAGgtagc (SEQ ID NO: 3). In still other embodiments of any aspect delineated herein, the probe has a fluorescent dye at 5′ end, and a quencher at 3′ end or vice versa. In still other embodiments of any aspect delineated herein, the probe is

(SEQ ID NO: 4)
5′-CALRed₆₁₀ ₙₘ-gctacACGACTTTYGCTGAAGgtagc BHQ2-3′
or
(SEQ ID NO: 5)
5′-FAM or FITC-gctacACGACTTTYGCTGAAGgtagc-BHQ1-3′.

In still other embodiments of any aspect delineated herein, the 3′ quencher is replaced by DABsyl.

In still other embodiments of any aspect delineated herein, the forward and reverse primers for detection of HIV virus contain one or more of the following sequences, respectively:

Forward primer:
(SEQ ID NO: 6)
GACTCGATATCGAGTCTGACTAGmCGGAGGmCmTmAmGmAmAmG,
(SEQ ID NO: 7)
GACTCGATATCGAGTCTGACTAGmCAGAGGmCmTmAmGmAmAmG;
and
Reverse Primer:
(SEQ ID NO: 8)
GACTCGATATCGAGTCTATTGACmGCTCmTmCmGmCmAmC,
(SEQ ID NO: 9)
GACTCGATATCGAGTCTACTGACmGCTCmTmCmGmCmAmC.

In still other embodiments of any aspect delineated herein, amplification is detected using a probe having the following sequence: cgcaagGGAGAGAGATGGGTGcttgcg (SEQ ID NO: 10).

In still other embodiments of any aspect delineated herein, the forward and reverse primers for detection of Dengue virus contain one or more of the following sequences, respectively:

Forward primer:
(SEQ ID NO: 11)
GACTCGATATCGAGTCCAAAAACmAGCATATTmGmAmCmGmC;
and
Reverse Primer:
(SEQ ID NO: 12)
GACTCGATATCGAGTCAGACAGCmAGGATCmTmCmTmGmG,
(SEQ ID NO: 13)
GACTCGATATCGAGTCAGACAGCmAGGATCmTmGmTmGmG.

In still other embodiments of any aspect delineated herein, amplification is detected using a probe having the following sequence: cgcatcTGGTCTTTCCCAGCgatgcg (SEQ ID NO: 14).

In still other embodiments of any aspect delineated herein, the forward and reverse primers for detection of influenza B virus contain one or more of the following sequences, respectively:

Forward primer:
(SEQ ID NO: 15)
GACTCGATATCGAGTCAAATGCAmGATGGTCTCmAmGmCmTmA,
(SEQ ID NO: 16)
GACTCGATATCGAGTCAAATGCAmAATGGTCTCmAmGmCmTmA,
(SEQ ID NO: 17)
GACTCGATATCGAGTCAAATGCAmGATGGTTTCmAmGmCmTmA;
and
Reverse Primer:
(SEQ ID NO: 18)
GACTCGATATCGAGTCCTCCTTTmTCCCATTCCATmTmCmAmTmT,
(SEQ ID NO: 19)
GACTCGATATCGAGTCCTCCCTTmTCCCATTCCATmTmCmAmTmT,
(SEQ ID NO: 20)
GACTCGATATCGAGTCCTCCTTTmCCCCATTCCATmTmCmAmTmT.

In still other embodiments of any aspect delineated herein, amplification is detected using a probe having the following sequence: gccaaGCTATGAACACAGCAAActtggc (SEQ ID NO: 21).

In still other embodiments of any aspect delineated herein, the forward and reverse primers for detection of BVDV1 virus contain one or more of the following sequences, respectively:

Forward primer:
(SEQ ID NO: 22)
GACTCGATATCGAGTCGGCCCACmTGTATTGCTmAmCmTmGmAmAmA,
(SEQ ID NO: 23)
GACTCGATATCGAGTCGGCCCACmTGCACTGCTmAmCmTmAmAmAmA;
and
Reverse Primer:
(SEQ ID NO: 24)
GACTCGATATCGAGTCTGTGATCmAACTCCmAmTmGmTmGmCmC.

In still other embodiments of any aspect delineated herein, amplification is detected using a probe having the following sequence: cgctacATCTCTGCTGTACATGgtagcg (SEQ ID NO: 25). In still other embodiments of any aspect delineated herein, the probe has a fluorescent dye at the 5′ end and a quencher at the 3′ end, or a fluorescent dye at the 3′ end and a quencher at the 5′ end. In particular embodiments, the fluorescent dye is CALRed₆₁₀ₙₘ, and the quencher is BHQ2 or DABsyl. In certain embodiments, the fluorescent dye is FAM or FITC and the quencher is BHQ1 or DABsyl.

In still other embodiments of any aspect delineated herein, the RNA virus is an Ebola virus, human immunodeficiency virus (HIV), Dengue virus, influenza virus (e.g., influenza B), Bovine Viral Diarrhea virus (e.g., BVDV Genotype 1), Yellow Fever virus, West Nile virus, Hepatitis C, Lassa virus, Flavivirus, Arenavirus, or single-stranded RNA virus. In still other embodiments of any aspect delineated herein, the agent capable of extracting the virus is one of or a combination of sodium dodecyl sulfate, sodium lauryl sulfate, Guanidinium thiocyanate, and/or guanidine hydrochloride. In various embodiments, the Guanidinium thiocyanate or other agent capable of extracting the virus is used at a concentration of about 0.1, 0.5, 1.0, 2.5, 5.0, 7.5, 10, 15, 20, 25, 50, 100, 250, 500 mM or more. In still other embodiments of any aspect delineated herein, the method is used for daily screening of health care workers. In still other embodiments of any aspect delineated herein, the samples are pooled and the screening is carried out on a human or animal population.

### Definitions

By “Ebola virus (EBOV)” is meant a Filoviridae virus having at least about 85% amino acid sequence identity to an Ebola virus. Exemplary Ebola viruses include, but are not limited to, Ebola-Zaire virus, Ebola-Sudan virus, Ebola-Ivory Coast virus, and Ebola-Bundibugyo, which cause disease in humans, or Ebola-Reston virus, which affects non-human primates.

The sequence of an exemplary Ebola Zaire genome is provided at NCBI Accession No. KC242800.1 (SEQ ID NO: 26), which is reproduced below:

| | |
|---|---|
| 1 | cggacacaca aaaagaaaga agaattttta ggatcttttg tgtgcgaata actatgagga |
| 61 | agattaataa ttttcctctc attgaaattt atatcggaat ttaaattgaa attgttactg |
| 121 | taatcacacc tggtttgttt cagagccaca tcacaaagat agagaacagc ctaggtctcc |
| 181 | gaagggaaca agggcaccag tgtgctcagt tgaaaatccc ttgtcaacat ctaggtctta |
| 241 | tcacatcaca agttccacct cagactctgc agggtgatcc aacaacccta atagaaaaat |
| 301 | tattgttaac ggacagcatt agttcacagt caaacaagca agattgagaa ttaaccttga |
| 361 | ttttgaactt caacacctag aggattggag attcaacaac cctaaaactt ggggtaaaac |
| 421 | attggaaata gttgaaagac aaattgctcg gaatcacaaa attccgagta tggattctcg |
| 481 | tcctcagaaa gtctggatga cgccgagtct tactgaatct gacatggatt accacaagat |
| 541 | cttgacagca ggtctgtccg ttcaacaggg gattgttcgg caaagagtca tcccagtgta |
| 601 | tcaagtaaac aatcttgagg aaatttgcca acttatcata caggcctttg aagcaggtgt |
| 661 | tgattttcaa gagagtgcgg acagtttcct tctcatgctt tgtcttcatc atgcgtacca |
| 721 | aggagatcac aaacttttct tggaaagtgg tgcagtcaag tatttggaag ggcacgggtt |
| 781 | ccgttttgaa gtcaagaaac gtgatggggt gaagcgcctt gaggaattgc tgccagcagt |
| 841 | atctagtgga aaaaacatta agagaacact tgctgccatg ccggaagagg agacgactga |
| 901 | agctaatgcc ggtcagtttc tctcttttgc aagtctattc cttccgaaat tggtagtagg |
| 961 | agaaaaggct tgccttgaga aagttcaaag gcaaattcaa gtacatgcag agcaaggact |
| 1021 | gatacaatat ccaacagctt ggcaatcagt aggacacatg atggtgattt tccgtttgat |
| 1081 | gcgaacaaat tttttgatca aatttctcct aatacaccaa gggatgcaca tggttgccgg |
| 1141 | gcatgatgcc aacgatgctg tgatttcaaa ttcagtggct caagctcgtt tttcaggttt |
| 1201 | attgattgtc aaaacagtcc ttgatcatat cctacaaaag acagaacgag gagttcgtct |
| 1261 | ccatcctctt gcaaggactg ccaaggtaaa aaatgaggtg aactccttta aggctgcact |
| 1321 | cagctccctg gccaagcatg gagagtatgc tcctttcgcc cgacttttga acctttctgg |
| 1381 | agtaaataat cttgagcatg gtcttttccc tcaactatcg gcaattgcac tcggagtcgc |
| 1441 | cacagcacac gggagcaccc tcgcaggagt aaatgttgga gaacagtatc aacagctcag |
| 1501 | agaggctgcc actgaagctg agaagcaact ccaacaatat gcagaatctc gcgaacttga |
| 1561 | ccatcttgga cttgatgatc aggaaaagaa aattcttatg aacttccatc agaaaaagaa |
| 1621 | cgaaatcagc ttccagcaaa caaacgctat ggtaactcta agaaaagagc gcctggccaa |
| 1681 | gctgacagaa gctatcactg ctgcatcact gcccaaaaca agtggacctt acgatgatga |
| 1741 | tgacgacatt ccctttccag gacccatcaa tgatgacgac aatcctggcc atcaagatga |
| 1801 | tgatccgact gactcacagg atacgaccat tcccgatgtg gtggttgatc ccgatgatgg |
| 1861 | aagctacggc gaataccaga gttactcgga aaacggcatg aatgcaccag atgacttggt |
| 1921 | cctattcgat ctagacgagg acgacgagga cactaagcca gtgcctaaca gattgaccaa |
| 1981 | gggtggacaa cagaaaaaca gtcaaaaggg ccagcataca gagggcagac agacacaatc |
| 2041 | caggccaact caaaatgtcc caggccctcg cagaacaatc caccacgcca gtgctccact |
| 2101 | cacggacaac gacagaggaa atgaaccctc cggctcaacc agccctcgca tgctgacacc |
| 2161 | aattaacgaa gaggcagacc cactggacga tgccgacgac gagacgtcta gtcttccgcc |
| 2221 | cttggagtca gacgatgaag aacaggacag ggacgaaact tccaaccgca cacccactgt |
| 2281 | cgccccaccg gctcccgtat acagagatca ctctgaaaag aaagaactcc cgcaagatga |
| 2341 | gcagcaagat caggaccaca ctcaagaggc caggaaccag gacagtgaca acacccagcc |
| 2401 | agaacactct tttgaggaga tgtatcgcca cattctaaga tcacagggac catttgatgc |
| 2461 | tgttttgtat tatcatatga tgaaggatga gcctgtagtt ttcagtacta gtgatggcaa |
| 2521 | agagtacacg tatccggact cccttgaaga ggaatatcca ccatggctca ctgaaaaaga |
| 2581 | ggccatgaat gaagagaata gatttgttac attggatggt caacaatttt attggccggt |
| 2641 | aatgaatcac aagaataaat tcatggcaat cctgcaacat catcagtgaa tgagaatgga |
| 2701 | ataatgggat gatttaaccg acaaatagct aacattaaat agtcaagaaa cgcaaacagg |
| 2761 | aagaattttt gatgtctaag gtgtgaatta ttatcacaat aaaagtgatt cttatttttg |
| 2821 | aatttaaagc tagcttatta ttactagccg tttttcaaag ttcaatttga gtcttaatgc |
| 2881 | aaataggcgt taagccacag ttatagccat aattgtaact caatatctta gctagcgatt |
| 2941 | tatctaaatt aaattacatt atgcttttat aacttaccta ctagcctgcc caacatttac |
| 3001 | acgatcgttt tataattaag aaaaaactaa tgatgaagat taaaaccttc atcatcctta |
| 3061 | cgtcaattga attctctagc actcgaagct tattgtcttc aatgtaaaag aaaagctggt |
| 3121 | ccaacaagat gacaactaga acaaagggca ggggccatac tgtggccacg actcaaaacg |
| 3181 | acagaatgcc aggccctgag ctttcgggct ggatctccga gcagctaatg accggaagaa |
| 3241 | ttcctgtaag cgacatcttc tgtgatattg agaacaatcc aggattatgt tacgcatccc |
| 3301 | aaatgcaaca aacaaagcca aacccgaaga tgcgcaacag tcaaacccaa acggacccaa |
| 3361 | tttgcaatca tagttttgag gaggtagtac aaacattggc ttcattggct actgttgtgc |
| 3421 | aacaacaaac tatcgcatca gaatcattag aacaacgtat tacgagtctt gagaatggtc |
| 3481 | taaagccagt ttatgatatg gcaaaaacaa tctcctcatt gaacagggtt tgtgctgaga |
| 3541 | tggttgcaaa atatgatctt ctggtgatga caaccggtcg ggcaacagca accactgcgg |
| 3601 | caactgaggc ttattgggct gaacatggtc aaccaccacc tggaccatca ctttatgaag |
| 3661 | aaagtgcaat tcggggtaag attgaatcta gagatgagac cgtccctcaa agtgttaggg |
| 3721 | aggcattcaa caatctagac agtaccactt cactaactga ggaaaatttt gggaaacctg |
| 3781 | acatttcagc aaaggatttg agaaacatta tgtatgatca cttgcctggt tttggaactg |
| 3841 | ctttccacca attagtacaa gtgatttgta aattgggaaa agatagcaac tcattggata |
| 3901 | tcattcatgc tgagttccag gccagcctgg ctgaaggaga ctctcctcaa tgtgccctaa |
| 3961 | ttcaaattac aaaaagagtt ccaatcttcc aagatgctgc tccacctgtc atccacatcc |
| 4021 | gctctcgagg tgacattccc cgagcttgcc agaaaagctt gcgtccagtc ccgccatcac |
| 4081 | ccaagattga tcgaggttgg gtatgtgttt tccagcttca agatggtaaa acacttggac |
| 4141 | tcaaaatttg agccaatctc ccttccctcc gaaagaggcg accaatagca gaggcttcaa |
| 4201 | ctgctgaact acagggtacg ttacattaat gatacacttg tgagtatcag ccctagataa |
| 4261 | tataagtcaa ttaaacgacc aagccaaaat tgttcatatc ccgctagcag cttaaaatat |
| 4321 | aaatgaaata ggagctatat ctctgacagt attataatca attgttatta agtaacccaa |
| 4381 | accaaaaatg atgaagatta agaaaaacct acctcgactg agagagtgtt tttccattaa |
| 4441 | ccttcatctt gtaaacgttg agcaaaattg ttacgaatat gaggcgggtt atattgccta |
| 4501 | ctgctcctcc tgaatatatg gaggccatat accctgtcag gtcaaattca acaattgcta |
| 4561 | ggggtggcaa caacaataca ggcttcctga caccggagtc agtcaatgga gacactccat |
| 4621 | cgaatccact caggccaatt gctgatgaca ccatcgacca tgctagccac acaccaggca |
| 4681 | gtgtgtcatc agcattcatc cttgaagcta tggtgaatgt catatcgggc cccaaagtgc |
| 4741 | taatgaagca aattccaatt tggcttcctc taggtgtcgc tgatcaaaag acctacagct |
| 4801 | ttgactcaac tacggccgcc atcatgcttg cttcatatac tatcacccat ttcggcaagg |
| 4861 | caaccaatcc acttgtcaga gtcaatcggc tgggtcctgg aatcccggat caccccctca |
| 4921 | ggctcctgcg aattggaaac caggccttcc tccaggagtt cgttcttccg ccagtccaac |
| 4981 | taccccagta tttcaccttt gatttgacag cactcaaact gatcacccaa ccactgcctg |
| 5041 | ctgcaacatg gaccgatgac actccaacag gatcaaatgg agcgctgcgt ccaggaattt |
| 5101 | cgtttcatcc aaaacttcgc cccattcttt tacctaacaa aagtgggaag aaggggaaca |
| 5161 | gtgccgatct aacatctcca gagaaaatcc aagcaataat gacttcactc caggacttta |
| 5221 | agatcgttcc aattgatcca accaaaaata tcatgggtat cgaagtgcca gaaactctgg |
| 5281 | tccacaagct gaccggtaag aaggtgactt ctaaaaatgg acaaccaatc atccctgttc |
| 5341 | ttttgccaaa gtacattggg ttggacccgg tggctccagg agacctcacc atggtaatca |
| 5401 | cacaggattg tgacacgtgt cattctcctg caagtcttcc agctgtgatt gagaagtaat |
| 5461 | tgcaataatt gactcagatc cagttttaca gaatcttctc agggatagtg ataacatcta |
| 5521 | tttagtaatc cgtctattag aggagatact tttaattgat caatatacta aaggtgcttt |
| 5581 | acaccattgt cttttttctc tcctaaatgt agaacttaac aaaagactca caatatactt |
| 5641 | gtcttaaaga gattgattga tgaaagatca tgactaataa cattacaaat aatcctacta |
| 5701 | taatcaatac ggtgattcaa atattaatct ttctaattgc acatactctc tgcccctatc |
| 5761 | ctcaaattgc ctacatgcct acatctgagg atagccagtg tgacttggat tggagatgta |
| 5821 | gggaagaaat cggaacccat ctccaggttg ttcacaatcc aagcacagac atcgcccttc |
| 5881 | taattaagaa aaaatcggcg atgaagatta agccgacagt gagcgcaatc ttcatctctc |
| 5941 | ttagattatt tgttttccag agtaggggtc atcaggtcct ttccaatcat ataaccaaaa |
| 6001 | taaacttcac tagaaggata ttgtgaggca acaacacaat gggtattaca ggaatattgc |
| 6061 | agttacctcg tgatcgattc aagaggacat cattctttct ttgggtaatt atccttttcc |
| 6121 | aaagaacatt ttccatccca cttggagtca tccacaatag cacattacaa gttagtgatg |
| 6181 | tcgacaaact agtttgtcgt gacaaactgt catccacaaa tcaattgaga tcagttggac |
| 6241 | tgaatctcga agggaatgga gtggcaactg acgtgccatc tgcaactaaa agatggggct |
| 6301 | tcaggtccgg tgtccctcca aaggtggtca attatgaagc tggtgaatgg gctgaaaact |
| 6361 | gctacaatct tgaaatcaaa aaacctgacg ggagtgagtg tctaccagca gcgccagacg |
| 6421 | ggattcgggg cttcccccgg tgccggtatg tgcacaaagt atcaggaacg ggaccgtgtg |
| 6481 | ccggagactt tgccttccac aaagagggtg ctttcttcct gtatgatcga cttgcttcca |
| 6541 | cagttatcta ccgaggaacg actttcgctg aaggtgtcgt tgcatttctg atactgcccc |
| 6601 | aagctaagaa ggacttcttc agctcacacc ccttgagaga gccggtcaat gcaacggagg |
| 6661 | acccgtccag tggctactat tctaccacaa ttagatatca ggctaccggt tttggaacca |
| 6721 | atgagacgga gtacttgttc gaggttgaca atttgaccta cgtccaactt gaatcaagat |
| 6781 | tcacgccaca gtttttgctc cagctgaatg agacaatata tgcaagtggg aaaaggagca |
| 6841 | acaccacggg aaaactaatt tggaaggtca accccgaaat tgatacaaca atcggggagt |
| 6901 | gggccttctg ggaaactaaa aaaacctcac tagaaaaatt cgcagtgaag agttgtcttt |
| 6961 | cacagctgta tcaaacggag ccaaagacat cagtggtcag agtccggcgc gaacttcttc |
| 7021 | cgacccagag acctacacaa caactgaaga ccacaaaatc atggcttcag aaaattcctc |
| 7081 | tgcaatggtt caagtgcaca atcaaggaag ggaagctgca gtgtcgcatc tgataaccct |
| 7141 | tgccacaatc tccacgagtc ctcaatcccc tacaaccaaa ccaggtcagg acaacagcac |
| 7201 | ccataataca cccgtgtata aacttgacat ctctgaggca actcaagttg aacaacatca |
| 7261 | tcgcagaaca gacaacgaca gcacagcctc cgacactccc cccgccacga ccgcagccgg |
| 7321 | acccccaaaa gcagagaaca tcaacacgag caagagcgct gactccctgg accccgccac |
| 7381 | cacgacaagt ccccaaaact acagcgagac cgctggcaac aacaacactc atcaccaaga |
| 7441 | taccggagaa gagagtgccg gcagcgggaa gctgggcttg attgccaata ctattgctgg |
| 7501 | agtcgcaggg ctgatcacag gcgggagaag aactcgaaga gaagcaattg tcaatgctca |
| 7561 | acccaaatgc aaccccaatc tacattactg gactactcag gatgaaggtg ctgcaatcgg |
| 7621 | attggcctgg ataccatatt tcgggccagc agccgaggga atttacacag aggggctaat |
| 7681 | gcacaatcaa gatggtttaa tctgtggatt gaggcagctg gccaatgaga cgactcaagc |
| 7741 | tcttcaactg ttcctgagag ccacaactga gctacgcacc ttttcaatcc tcaaccgtaa |
| 7801 | ggcaattgat ttcttgctgc agcgatgggg cggcacatgc cacattttgg gaccggactg |
| 7861 | ctgtatcgaa ccacatgatt ggaccaagaa cataacagac aaaattgatc agattattca |
| 7921 | tgattttgtt gataaaaccc ttccggacca gggggacaat gacaattggt ggactggatg |
| 7981 | gagacaatgg ataccggcag gtattggagt tacaggcgtt ataattgcag ttattgcttt |
| 8041 | attctgtata tgcaaatttg tcttttagtt tttcttcaga ttgcttcatg gcaaagctca |
| 8101 | gcctcaaatc aatgagatta ggatttaatt atatggatca cttgaatcta agattacttg |
| 8161 | acaaatgata atataataca ctggagcttt aaatatagcc aatgtgattc taactccttt |
| 8221 | aaactcacaa ttaatcataa acaaggtttg acatcaatct agttatatct ttgagaatga |
| 8281 | taaacttgat gaagattaag aaaaaggtaa tctttcgatt atctttagtc ttcatccttg |
| 8341 | attctacaat catgacagtt gtctttagtg acaagggaaa gaagcctttt tagtaagttg |
| 8401 | taataatcag atctgcgaac cggtagagtt taattgcaac ctaacacaca taaagcattg |
| 8461 | gtcaaaaagt caatagaaat ttaaacagtg agtggagaca actttcaaat ggaagctcca |
| 8521 | tacgagagag gacgcccccg agctgccaga cagcattcaa gggatggaca cgaccatcat |
| 8581 | gttcgagcac gatcatcatc cagagagaat tatcgaggtg agtaccgtca atcaaggagc |
| 8641 | gcctcacaag tgcgcgttcc tactgtattt cataagagga gagttgaacc attaacagtt |
| 8701 | cctccagcac ctaaagacat atgtccgacc ttgaaaaaag gatttttgtg tgacagtagt |
| 8761 | ttttgcaaaa aagatcacca gttggaaagt ttaactgata gggaattact cctactaatc |
| 8821 | gcccgtaaga cttgtggatc agtagaacaa caattaaata taactgcacc caaggactcg |
| 8881 | cgcttagcaa atccaacggc tgatgatttc cagcaagagg aaggtccaaa aattaccttg |
| 8941 | ttgacactga tcaagacggc agaacactgg gcgagacaag acatcaggac cacagaggat |
| 9001 | tcaaaattaa gagcattgtt gactctatgt gctgtgatga cgaggaaatt ctcaaaatcc |
| 9061 | cagctgagtc ttttatgtga gacacacctg aggcgcgagg ggcttgggca agatcaggca |
| 9121 | gaacccgttc tcgaagtata tcaacgatta cacagtgata aaggaggcag tttcgaagct |
| 9181 | gcactatggc aacaatggga tcgacaatcc ctaattatgt ttatcactgc attcttgaat |
| 9241 | atcgctctcc agttaccgtg tgaaagttct gctgtcgttg tttcagggtt aagaacattg |
| 9301 | gttcctcaat cagataatga ggaagcttca accaacccgg ggacatgctc atggtctgat |
| 9361 | gatggtaccc cttaataagg ctgactaaaa cactatataa ccttctactt gatcacaata |
| 9421 | ctccgtatac ctatcatcat atattcaatc aagacggtat cctttaaaac ttattcagta |
| 9481 | ctataatcac tctcgtttca aattaataag atatgcataa ttgctttaat atatgaagag |
| 9541 | gtatgataca accctaacag tgatcaaaga aaatcataat ctcttatcgc tcgtaatata |
| 9601 | acctgccaag catacctctt gcacaaagtg attcttgtac acaaataatg ttttactcta |
| 9661 | caggaggtag caacgatcca tcccatcaaa aaataagtat tttatgactt actaatgatc |
| 9721 | tcttaaaata ttaagaaaaa ctgacggaac acaaattctt tctgcttcaa gttgtggagg |
| 9781 | aggtctttgg tattggctat tgttatatta caatcaataa caagcttgta aaaatattgt |
| 9841 | tcttgtttca agaggtagat tgtgaccgga aacgctaaac taatgatgaa gattaatgcg |
| 9901 | gaggtctgat aagaataaac cttattattc agattaggcc ccaagaggca ttcttcatct |
| 9961 | ccttttagca aagtactatt tcagggtagt ccaattagtg acacgtcttt tagctgtata |
| 10021 | tcagtcgccc ctgagatacg ccacaaaagt gtctctaagc taaattggtc tgtacacatc |
| 10081 | tcatacattg tattaggggc aataatatct aattgaactt agccgtttaa aatttagtgc |
| 10141 | ataaacctgg gctaactcca ccaggtcaac tccattggct gaaaagaagc ccacctacaa |
| 10201 | cgaacatcac tttgagcgcc cttacaatta aaaaatagga acgtcgttcc aacaattgag |
| 10261 | cgcaaggttt caaggttgaa ctgagagtgc ctaaacacca aaatatcgat aattcagaca |
| 10321 | ccaagcaaga cctgagaagg aaccatggct aaagctacgg gacgatacaa tctaatatcg |
| 10381 | cccaaaaagg acctggagaa aggggttgtc ttaagcgacc tctgtaactt cctagttagt |
| 10441 | caaactattc aagggtggaa ggtctattgg gctggtattg agtttgatgt gactcacaaa |
| 10501 | ggaatggccc tattgcatag actgaaaact aatgactttg cccctgcatg gtcaatgaca |
| 10561 | aggaatctat ttcctcattt atttcaaaat ccgaattcca caattgagtc accactgtgg |
| 10621 | gcattgagag tcatccttgc agcaggggta caggaccagc tgattgacca gtctttgatt |
| 10681 | gaacccttag caggagccct tggtctgatc tctgattggc tgctaacaac caacactaac |
| 10741 | catttcaaca tgcgaacaca acgtgttaag gaacaattga gcctaaaaat gctgtcgttg |
| 10801 | attcgatcca atattctcaa gtttattaac caattggatg ctctacatgt cgtgaactac |
| 10861 | aacgggttgt tgagcagtat tgaaattgga actcaaaatc atacaatcat tataactcga |
| 10921 | actaacatgg gttttctggt ggagctccaa gaacccgaca aatcggcaat gaaccgcaag |
| 10981 | aagcctgggc cggcgaaatt ttccctcctt catgagtcca cactgaaagc atttacacaa |
| 11041 | gggtcctcga cacgaatgca aagtttgatt cttgaattta atagctctct tgctatctaa |
| 11101 | ttaagatgga atacttcata ttgagctaac tcatatatgc tgactcaata gttatcttga |
| 11161 | catctctgct ttcataatca gatatataag cataataaat aaatactcat atttcttgat |
| 11221 | aatttgttta accacagata aatcctaact gtaagccagc ttccaagttg acacccttac |
| 11281 | aaaaaccagg actcagaatc cctcaaataa gagattccaa gacaacatca tagaattgct |
| 11341 | ttattatatg aataagcatg ttatcaccag aaatccaata tactaaatag ttaattgtaa |
| 11401 | ctgaacccgc aggtcacgtg tgttaggttt cacagattat atatattact aactccatac |
| 11461 | ccgtaattaa cattagataa gtagattaag aaaaacgctt gaggaagatt aagaaaaact |
| 11521 | gcttattggg tctttccgtg ttttagatga agcagttgac attcttcctc ttgatattaa |
| 11581 | atggctacac aacataccca atacccagac gccaggttat catcaccaat tgtattggac |
| 11641 | caatgtgacc tagtcactag agcttgcggg ttatattcat catactccct taatccgcaa |
| 11701 | ctacgcaact gtaaactccc gaaacatatc taccgtttaa aatatgatgt aactgttacc |
| 11761 | aagttcttaa gtgatgtacc agtggcgaca ttgccaatag atttcatagt cccaattctt |
| 11821 | ctcaaggcac tgtcaggcaa tgggttctgt cctgttgagc cgcggtgtca acagttctta |
| 11881 | gatgaaatca ttaagtacac aatgcaagat gctctcttcc tgaaatatta tctcaaaaat |
| 11941 | gtgggtgctc aagaggactg tgttgatgac cactttcaag agaaaatctt atcttcaatt |
| 12001 | cagggcaatg aatttttaca tcaaatgttc ttctggtatg acctggctat tttgactcga |
| 12061 | aggggtagat taaatcgagg aaactctaga tcaacatggt ttgttcatga tgatttaata |
| 12121 | gacatcttag gctatgggga ctatgttttt tggaagatcc caatttcaat gttacccctg |
| 12181 | aacacacaag gaatccccca tgctgctatg gattggtatc aggcatcagt attcaaagaa |
| 12241 | gcggttcaag ggcatacaca cattgtttct gtttctactg ccgacgtctt gataatgtgc |
| 12301 | aaagatttaa ttacatgtcg attcaacaca actctaatct caaagatagc agaggttgag |
| 12361 | gatccagttt gttctgatta tcccgatttt aagattgtgt ctatgcttta ccagagcgga |
| 12421 | gattacttac tctccatatt agggtctgat gggtataaaa ttattaagtt cctcgaacca |
| 12481 | ttgtgcttgg ccaaaattca attatgctca aagtacaccg agaggaaggg ccgattctta |
| 12541 | acacaaatgc atttagctgt aaatcacacc ctggaagaaa ttacagaaat gcgtgcacta |
| 12601 | aagccttcac aggatcaaaa gatccgtgaa ttccatagaa cattgataag gctggagatg |
| 12661 | acgccacaac aactttgtga gctattttcc attcaaaaac actgggggca tcctgtgcta |
| 12721 | catagtgaaa cagcaatcca aaaagttaaa aaacatgcca cggtgctaaa agcattacgc |
| 12781 | cctatagtga ttttcgagac atattgtgtt tttaaatata gtattgcaaa acattatttt |
| 12841 | gatagtcaag gatcttggta cagtgttact tcagatagga atttaacgcc aggtcttaat |
| 12901 | tcttatatca aaagaaatca attccccccg ttgccaatga ttaaagaact actatgggaa |
| 12961 | ttttaccacc ttgaccatcc tccacttttc tcaaccaaaa ttattagtga cttaagtatt |
| 13021 | tttataaaag acagagctac cgcagtggaa aggacatgct gggatgcagt attcgagcct |
| 13081 | aatgttctag gatataatcc acctcacaaa ttcagtacta aacgtgtacc agaacaattt |
| 13141 | ttagagcaag aaaacttttc tattgagaat gttctttcct acgcgcaaaa actcgagtat |
| 13201 | ctactaccac aataccggaa tttttctttc tcattgaaag agaaagagtt gaatgtaggt |
| 13261 | agaactttcg gaaaattgcc ttatccgact cgcaatgttc aaacactttg tgaagctctg |
| 13321 | ttagctgatg gtcttgctaa agcatttcct agcaatatga tggtagtcac agagcgtgag |
| 13381 | caaaaagaaa gcttattgca tcaagcatca tggcaccaca caagtgatga ttttggtgag |
| 13441 | catgccacag ttagagggag tagctttgta actgatttag agaaatacaa tcttgcattt |
| 13501 | agatatgagt ttacagcacc ttttatagaa tattgtaacc gttgctatgg tgttaagaat |
| 13561 | gtttttaatt ggatgcatta tacaatcccc cagtgttata tgcatgtcag tgattattat |
| 13621 | aatccaccgc ataacctcac tctggaaaat cgagacaacc cccccgaagg gcccagttca |
| 13681 | tacagaggtc atatgggagg gattgaagga ctgcaacaaa aactctggac aagtatttca |
| 13741 | tgtgctcaaa tttctttagt tgaaataaag actggtttta agttacgctc agctgtgatg |
| 13801 | ggtgacaatc agtgcattac cgttttatca gtcttcccct tagagactga cgcagacgag |
| 13861 | caggaacaga gcgccgaaga caatgcagcg agggtggccg ccagcctagc aaaagttaca |
| 13921 | agtgcctgtg gaatcttttt aaaacctgat gaaacatttg tacattcagg ttttatctat |
| 13981 | tttggaaaaa aacaatattt gaatggggtc caattgcctc agtcccttaa aacggctaca |
| 14041 | agaatggcac cattgtctga tgcaattttt gatgatcttc aagggaccct ggctagtata |
| 14101 | ggcactgctt ttgaacgatc catctctgag acacgacata tctttccttg caggataacc |
| 14161 | gcagctttcc atacgttttt ttcggtgaga atcttgcaac atcatcacct cgggttcaat |
| 14221 | aagggttttg accttggaca gttgacactt ggcaaacctc tggatttcgg aacaatatca |
| 14281 | ttggcactag cggtaccgca ggtgcttgga gggttatcct tcttgaatcc tgagaaatgt |
| 14341 | ttctaccgga atttaggaga tccagttacc tcaggcttat tccagttaaa aacttatctc |
| 14401 | cgaatgattg agatggatga tttattctta cctttaattg cgaagaaccc tgggaactgc |
| 14461 | actgccattg actttgtgct aaatcctagc ggattaaatg tccccgggtc gcaagactta |
| 14521 | acttcatttc tgcgccagat tgtgcgtagg actatcaccc taagtgcgaa aaacaaactt |
| 14581 | attaatactt tatttcatgc gtcagctgac ttcgaagacg aaatggtttg taaatggcta |
| 14641 | ttatcatcaa ctcctgttat gagtcgtttt gcggccgata tcttttcacg cacgcccagt |
| 14701 | gggaagcgat tgcaaattct aggatacctg gaaggaacac gcacattatt agcctctaag |
| 14761 | atcatcaaca ataatacaga aacaccggtt ttggacagac tgaggaaaat aacattgcaa |
| 14821 | aggtggagtc tatggtttag ttatcttgat cattgtgata atatcctggc agaggcttta |
| 14881 | acccaaataa cttgcacagt tgatttagca cagatcctga gggaatattc atgggcacat |
| 14941 | attttagagg ggagacctct tattggagcc acacttccat gtatgattga gcaattcaaa |
| 15001 | gtggtttggc tgaaacccta cgaacaatgt ccgcagtgtt caaatgcaaa gcaacctggt |
| 15061 | gggaaaccat tcgtgtcagt ggcagtcaag aaacatattg ttagtgcatg gccgaacgca |
| 15121 | tcccgaataa gctggactat cggggatgga atcccataca ttggatcaag gacagaagat |
| 15181 | aagataggac aacctgctat taaaccaaaa tgtccttccg cagccttaag agaggccatt |
| 15241 | gaactggcgt cccgtttaac atgggtaact caaggcagtt cgaacagtga tttgctaata |
| 15301 | aaaccatttt tggaagcacg agtaaattta agtgttcaag aaatacttca aatgacccct |
| 15361 | tcacattact caggaaatat tgttcacagg tacaacgatc aatatagtcc tcattctttc |
| 15421 | atggccaatc gtatgagtaa ttcagcgacg cgattgattg tttctactaa cactttaggt |
| 15481 | gagttttcag gaggtggcca gtctgcacgc gacagcaata ttattttcca gaatgttata |
| 15541 | aattatgcag ttgcactgtt cgatattaaa tttagaaaca ctgaggctac agatatccaa |
| 15601 | tataatcgtg ctcaccttca tctaactaag tgttgcaccc gggaagtacc agctcagtat |
| 15661 | ttaacataca catctacatt ggatttagat ttaacaagat accgagaaaa cgaattgatt |
| 15721 | tatgacaata atcctctaaa aggaggactc aattgcaata tctcattcga taacccattt |
| 15781 | ttccaaggta aacggctaaa cattatagaa gatgatctta ttcgactgcc tcacttatct |
| 15841 | ggatgggagc tagccaagac catcatgcaa tcaattattt cagatagcaa caattcgtct |
| 15901 | acagacccaa ttagcagtgg agaaacaaga tcattcacta cccatttctt aacttatccc |
| 15961 | aagataggac ttctgtacag ttttggggcc tttataagtt attatcttgg caatacaatt |
| 16021 | cttcggacta agaaattaac acttgacaat tttttatatt acttaactac ccaaattcat |
| 16081 | aatctaccac atcgctcatt gcgaatactt aagccaacat tcaaacatgc aagcgttatg |
| 16141 | tcacggttaa tgagtattga tcctcatttt tctatttaca taggcggtgc ggcaggtgac |
| 16201 | agaggactct cagatgcggc caggttattt ttgagaacgt ccatttcatc ttttcttgca |
| 16261 | tttataaaag agtggataat taatcgcgga acaattgtcc ctttatggat agtatatccg |
| 16321 | ctagagggtc aaaacccaac acctgttaat aatttcctcc atcagatcgt agaactgctg |
| 16381 | gtgcatgatt catcaagaca acaggctttt aaaactacca taagtgatca tgtacatcct |
| 16441 | cacgacaatc ttgtttacac atgtaagagt acagccagca atttcttcca tgcgtcattg |
| 16501 | gcgtactgga gaagcaggca cagaaacagc aatcgaaaat acttggcaag agactcttca |
| 16561 | actggatcaa gcacaaacaa cagtgatggt catattgaga gaagtcaaga acaaaccacc |
| 16621 | agagatccac atgatggcac tgaacggaat ctagtcctac aaatgagcca tgaaataaaa |
| 16681 | agaacgacaa ttccacaaga aagcacgcac cagggtccgt cgttccagtc atttctaagt |
| 16741 | gactctgctt gtggtacagc aaatccaaaa ctaaatttcg atagatcgag acataatgtg |
| 16801 | aaatctcagg atcataactc ggcatccaag agggaaggtc atcaaataat ctcacaccgt |
| 16861 | ctagtcctac ctttctttac attgtctcaa gggacgcgcc aattaacgtc atccaatgag |
| 16921 | tcacaaaccc aagacgagat atcaaagtac ttacggcaat tgagatccgt cattgatacc |
| 16981 | acagtttatt gtaggtttac cggtatagtc tcgtccatgc attacaaact tgatgaggtc |
| 17041 | ctttgggaaa tagagagttt taagtcggct gtgacgctag cagagggaga aggtgctggt |
| 17101 | gccttactat tgattcagaa ataccaagtt aagaccttat ttttcaacac gctagctact |
| 17161 | gagtccagta tagagtcaga aatagtatca ggaacgacta ctcctaggat gcttctacct |
| 17221 | gttatgtcaa aattccataa tgaccaaatt gagattattc ttaacaattc ggcaagccaa |
| 17281 | ataacagaca taacaaatcc tacttggttc aaagaccaaa gagcaaggct acctaggcaa |
| 17341 | gtcgaggtta taaccatgga tgcagagacg acagaaaata taaacagatc gaaattgtac |
| 17401 | gaagctgtat ataaattgat cttacaccat attgatccca gcgtattgaa agcagtggtc |
| 17461 | cttaaagtct ttctaagtga tactgagggt atgttatggc taaatgataa tttagccccg |
| 17521 | ttttttgcca ctggttattt aattaagcca ataacgtcaa gtgctagatc tagtgagtgg |
| 17581 | tatctttgtc tgacgaactt cttatcaact acacgtaaga tgccacacca aaaccatctc |
| 17641 | agttgtaaac aggtaatact tacggcattg caactgcaaa ttcaacggag cccatactgg |
| 17701 | ctaagtcatt taactcagta tgctgactgc gatttacatt taagttatat ccgccttggt |
| 17761 | tttccatcat tagagaaagt actataccac aggtataacc tcgtcgattc aaaaagaggt |
| 17821 | ccactagtct ctatcactca gcacttggca catcttagag cagagattcg agaattgact |
| 17881 | aatgattata atcaacagcg acaaagtcgg actcaaacat atcactttat tcgtactgca |
| 17941 | aaaggacgaa tcacaaaact agtcaatgat tatttaaaat tctttcttat tgtgcaagca |
| 18001 | ttaaaacata atgggacatg gcaagctgag tttaagaaat taccagagtt gattagtgtg |
| 18061 | tgcaataggt tctatcatat tagagattgc aattgtgaag aacgtttctt agttcaaacc |
| 18121 | ttatatctac atagaatgca ggattctgaa gttaagctta tcgaaaggct gacagggctt |
| 18181 | ctgagtttat tcccggatgg tctctacagg tttgattgaa ttaccgtgca tagtatcctg |
| 18241 | atacttgtga aggttgatta tcaacgtaca gattataaaa aactcacaaa ttgctctcat |
| 18301 | acatcatatt gatcgaattt caataaataa ctatttaaat aacgaaagaa gtccttatat |
| 18361 | tatacactat atttagcctc tctccctgcg tgataatcaa aaaattcaca atgcagcatg |
| 18421 | tgtgacatat tacttccgcg atgaatctaa cgcaacataa taaactctgc actctttata |
| 18481 | attaagcttt aacaaaaggt ctgggctcat attgttattg atataataat gttgtatcaa |
| 18541 | tatcctgtca gatggaatag tgttttggtt gataacacga cttcttaaaa caaaattgat |
| 18601 | cttcaagatt aagtttttta taattatcat tactttaatt tgtcgattta aaaatggtga |
| 18661 | tagccttaat ctttgtgtaa aataagagat taggtgtaat aactttaaca ttttgtctag |
| 18721 | taagctacta tttcatacag aatgataaaa ttaaaagaaa aggcatgact gtaaaatcag |
| 18781 | aaataccttc tttacaatat agcagactag ataataatct tcgtgttaat gataattaag |
| 18841 | acattgacca cgctcatcag gaggctcgcc aggataaacg ttgcaaaaag gattcctgga |
| 18901 | aaaatggtcg cacacaaaaa tttaaaaata aatctatttc ttcttttttg tgtgtcca |

The invention further provides polynucleotides having at least about 85, 90, 95, 96, 97, 98, 99, or 100% identity to this sequence. Other Ebola Zaire genomes are known in the art and described, for example, by Baize et al., N Engl J Med 2014; 371:1418-25, which is incorporated herein by reference.

By “Ribonuclease P RNA component H1 (RPPH1) is meant the RNA component of the RNase P ribonucleoprotein, an endoribonuclease that cleaves tRNA precursor molecules to form the mature 5-prime termini of their tRNA sequences. An exemplary nucleic acid sequence is provided at NCBI Accession No. NR_002312 (SEQ ID NO: 27).

| | |
|---|---|
| 1 | atagggcgga gggaagctca tcagtggggc cacgagctga gtgcgtcctg tcactccact |
| 61 | cccatgtccc ttgggaaggt ctgagactag ggccagaggc ggccctaaca gggctctccc |
| 121 | tgagcttcgg ggaggtgagt tcccagagaa cggggctccg cgcgaggtca gactgggcag |
| 181 | gagatgccgt ggaccccgcc cttcggggag gggcccggcg gatgcctcct ttgccggagc |
| 241 | ttggaacaga ctcacggcca gcgaagtgag ttcaatggct gaggtgaggt accccgcagg |
| 301 | ggacctcata acccaattca gactactctc ctccgcccat t |

By “amplicon” is meant a polynucleotide generated during the amplification of a polynucleotide of interest. In one example, an amplicon is generated during a polymerase chain reaction.

By “amplification rate modifiers” is meant an agent capable of affecting the rate of polymerase extension.

By “base substitution” is meant a substituent of a nucleobase polymer that does not cause significant disruption of the hybridization between complementary nucleotide strands.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

By “complementary” or “complementarity” is meant that a nucleic acid can form hydrogen bond(s) with another nucleic acid sequence by either traditional Watson-Crick or Hoogsteen base pairing. Complementary base pairing includes not only G-C and A-T base pairing, but also includes base pairing involving universal bases, such as inosine. A percent complementarity indicates the percentage of contiguous residues in a nucleic acid molecule that can form hydrogen bonds (e.g., Watson-Crick base pairing) with a second nucleic acid sequence (e.g., 5, 6, 7, 8, 9, or 10 nucleotides out of a total of 10 nucleotides in the first oligonucleotide being based paired to a second nucleic acid sequence having 10 nucleotides represents 50%, 60%, 70%, 80%, 90%, and 100% complementary respectively). To determine that a percent complementarity is of at least a certain percentage, the percentage of contiguous residues in a nucleic acid molecule that can form hydrogen bonds (e.g., Watson-Crick base pairing) with a second nucleic acid sequence is calculated and rounded to the nearest whole number (e.g., 12, 13, 14, 15, 16, or 17 nucleotides out of a total of 23 nucleotides in the first oligonucleotide being based paired to a second nucleic acid sequence having 23 nucleotides represents 52%, 57%, 61%, 65%, 70%, and 74%, respectively; and has at least 50%, 50%, 60%, 60%, 70%, and 70% complementarity, respectively). As used herein, “substantially complementary” refers to complementarity between the strands such that they are capable of hybridizing under biological conditions. Substantially complementary sequences have 60%, 70%, 80%, 90%, 95%, or even 100% complementarity. Additionally, techniques to determine if two strands are capable of hybridizing under biological conditions by examining their nucleotide sequences are well known in the art.

As used herein, “duplex” refers to a double helical structure formed by the interaction of two single stranded nucleic acids. A duplex is typically formed by the pairwise hydrogen bonding of bases, i.e., “base pairing”, between two single stranded nucleic acids which are oriented antiparallel with respect to each other. Base pairing in duplexes generally occurs by Watson-Crick base pairing, e.g., guanine (G) forms a base pair with cytosine (C) in DNA and RNA, adenine (A) forms a base pair with thymine (T) in DNA, and adenine (A) forms a base pair with uracil (U) in RNA. Conditions under which base pairs can form include physiological or biologically relevant conditions (e.g., intracellular: pH 7.2, 140 mM potassium ion; extracellular pH 7.4, 145 mM sodium ion). Furthermore, duplexes are stabilized by stacking interactions between adjacent nucleotides. As used herein, a duplex may be established or maintained by base pairing or by stacking interactions. A duplex is formed by two complementary nucleic acid strands, which may be substantially complementary or fully complementary. Single-stranded nucleic acids that base pair over a number of bases are said to “hybridize.”

“Detect” refers to identifying the presence, absence or amount of the analyte to be detected. In one embodiment, the analyte is an Ebola polynucleotide or other RNA viral polynucleotide.

By “detectable moiety” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “fragment” is meant a portion of a nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 5, 10, 15, 20, 30, 40, 50, 60, 70, 80, 90, or 100 nucleotides. In one embodiment, the fragment comprises at least about 50, 75, 80, 85, 89, 90, or 100 nucleotides of an Ebola polynucleotide or other RNA viral polynucleotide.

By “free energy (ΔG)” is meant the net exchange of energy between the system and its environment at a constant temperature and pressure described by the formula: ΔG=ΔH−TΔS. Free energy represents how thermodynamically stable a structure is, with formation of structures having a negative ΔG (e.g., expressed in kcal/mole) being thermodynamically stable (i.e., a structure having a lower ΔG is more stable than one having a higher ΔG). The thermodynamic potential is minimized when a system reaches equilibrium at constant pressure and temperature.

By “hybridize” is meant to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507). Hybridization occurs by hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “isolated polynucleotide” is meant a nucleic acid (e.g., a DNA, RNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purined” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “melting temperature (Tm)” is meant the temperature of a system in equilibrium where 50% of the molecular population is in one state and 50% of the population is in another state. With regard to the nucleic acids of the invention, Tm is the temperature at which 50% of the population is single-stranded and 50% is double-stranded (e.g., intramolecularly or intermolecularly).

By “monitoring a reaction” is meant detecting the progress of a reaction. In one embodiment, monitoring reaction progression involves detecting polymerase extension and/or detecting the completion of an amplification reaction.

As used herein, “obtaining” as in “obtaining an agent” includes synthesizing, purchasing, or otherwise acquiring the agent.

As used herein, the term “nucleic acid” refers to deoxyribonucleotides, ribonucleotides, or modified nucleotides, and polymers thereof in single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, 2′ modified nucleotides (e.g., 2′-O-methyl ribonucleotides, 2′-F nucleotides).

As used herein, “modified nucleotide” refers to a nucleotide that has one or more modifications to the nucleoside, the nucleobase, pentose ring, or phosphate group. For example, modified nucleotides exclude ribonucleotides containing adenosine monophosphate, guanosine monophosphate, uridine monophosphate, and cytidine monophosphate and deoxyribonucleotides containing deoxyadenosine monophosphate, deoxyguanosine monophosphate, deoxythymidine monophosphate, and deoxycytidine monophosphate. Modifications include those naturally occurring that result from modification by enzymes that modify nucleotides, such as methyltransferases. Modified nucleotides also include synthetic or non-naturally occurring nucleotides. Synthetic or non-naturally occurring modifications in nucleotides include those with 2′ modifications, e.g., 2′-O-methyl, 2′-methoxyethoxy, 2′-fluoro, 2′-hydroxyl (RNA), 2′-allyl, 2′-O-[2-(methylamino)-2-oxoethyl], 4′-thio, 4′-CH₂—O-2′-bridge, 4′-(CH₂)₂-O-2′-bridge, and 2′-O—(N-methylcarbamate) or those comprising base analogs.

By “nucleotide adduct” is meant a moiety that is bound covalently or otherwise fixed to a standard nucleotide base.

By “nicking agent” is meant a chemical entity capable of recognizing and binding to a specific structure in double stranded nucleic acid molecules and breaking a phosphodiester bond between adjoining nucleotides on a single strand upon binding to its recognized specific structure, thereby creating a free 3′-hydroxyl group on the terminal nucleotide preceding the nick site. In preferred embodiments, the 3′ end can be extended by an exonuclease deficient polymerase. Exemplary nicking agents include nicking enzymes, RNAzymes, DNAzymes, and transition metal chelators.

By “palindromic” is meant nucleic acid sequences that are identical or substantially identical when read from 5′ to 3′ on one strand or 5′ to 3′ on the complementary strand. A perfect palindrome refers to a sequence having two adjacent subsequences, such that when one subsequence is read from the 5′ to 3′ direction, it is identical to the other subsequence read from the 3′ to 5′ direction.

By “polymerase-arresting molecule” is meant a moiety associated with a polynucleotide template/primer that prevents or significantly reduces the progression of a polymerase on the polynucleotide template. Preferably, the moiety is incorporated into the polynucleotide. In one preferred embodiment, the moiety prevents the polymerase from progressing on the template.

By “polymerase extension” is meant the forward progression of a polymerase that matches incoming monomers to their binding partners on a template polynucleotide.

As used herein, “primer-dimer” is meant a dimer of two monomer oligonucleotide primers. In the oligonucleotide primers of the invention, the 5′ tail regions of monomer primers dimerize.

By “semi-quantitative” is meant providing an estimate of relative quantity based on an internal control.

By “specific product” is meant a polynucleotide product resulting from the hybridization of primer oligonucleotides to a complementary target sequence and subsequent polymerase mediated extension of the target sequence.

By “substantially isothermal condition” is meant at a single temperature or within a narrow range of temperatures that does not vary significantly. In one embodiment, a reaction carried out under substantially isothermal conditions is carried out at a temperature that varies by only about 1-5° C. (e.g., varying by 1, 2, 3, 4, or 5 degrees). In another embodiment, the reaction is carried out at a single temperature within the operating parameters of the instrument utilized.

By “quantity threshold method” is meant providing an estimate of quantity based on either exceeding or not exceeding in quantity a comparative standard.

By “reference” is meant a standard or control condition. As is apparent to one skilled in the art, an appropriate reference is where an element is changed in order to determine the effect of the element.

By “reverse transcriptase” is meant an enzyme that replicates a primed single-stranded RNA template strand into a complementary DNA strand in the presence of deoxyribonucleotides and permissive reaction medium comprising, but not limited to, a buffer (pH 7.0-9.0), sodium and/or potassium ions and magnesium ions. As is apparent to one skilled in the art, concentration and pH ranges of a permissive reaction media may vary in regard to a particular reverse transcriptase enzyme. Examples of suitable “reverse transcriptases” well known in the art, but not limited to, are MmLV reverse transcriptase and its commercial derivatives “Superscript I, II and III” (Life Technologies), “MaxiScript” (Fermentas), RSV reverse transcriptase and its commercial derivative “OmniScript” (Qiagen), AMV reverse transcriptase and its commercial derivative “Thermoscript” (Sigma-Aldrich).

By “subject” is meant a mammal, including, but not limited to, a human or non-human mammal, such as a bovine, equine, canine, ovine, or feline.

By “target nucleic acid molecule” is meant a polynucleotide to be analyzed. Such polynucleotide may be a sense or antisense strand of the target sequence. The term “target nucleic acid molecule” also refers to amplicons of the original target sequence.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIG. 1 is a schematic diagram illustrating the design of candidate assays for Ebola virus (EBOV). Assays 1 and 2 were tested for their ability to detect an EBOV polynucleotide (SEQ ID NO: 51).

FIG. 2 shows the various probes and primers tested (SEQ ID NOs: 52-65, respectively, in order of appearance), as well as the Ebola gBlock sequence used in assay development (SEQ ID NO: 66). A gBlock is a synthetic, double-stranded fragment of DNA ranging in size from 100 bp to 1000 bp that is entirely assembled in-vitro from synthetic single-stranded oligonucleotides and which represents a sequence-verified synthetic block of genomic DNA. Unless specifically stated or obvious from context, as used herein, the “gBlock®” is used as a synthetic target nucleic acid whenever a native target sequence or target genome is not available and/or not useful. Sequence-verified blocks of genomic DNA for any target sequence can be procured as a custom-order service from IDT Technologies Inc. (1710 Commercial Park, Coralville, Iowa 52241, USA) and the term “gBlock®” is a registered trademark of that company.

FIG. 3A-3X show amplification results obtained with Assay 1 and Assay 2.

FIG. 4 shows detection of Ebola virus in a background of total human RNA in a one-step assay.

FIG. 5 shows detection of synthetic Ebola virus RNA in a background of total human RNA in a one-step assay.

FIG. 6A-6C provide flow charts illustrating sample processing for use in an amplification and detection instrument, including, for example, a hand-held device. FIG. 6A indicates that a stabilization/lysis reagent (e.g., Guanidine isothiocyanate (GITC)) is lyophilized onto the inside of the capillary tube. FIG. 6B illustrates use of a paper impregnated with lyophilized GITC and buffer (e.g., Whatman™ FTA™ Elute Cards). FIG. 6C depicts a lyophilized assay procedure in multiplex (e.g., an 8-well strip) or single tube.

FIGS. 7A and 7B show sample preparation methods. FIG. 7A depicts sample preparation from swab samples. FIG. 7B depicts sample preparation from a blood sample.

FIG. 8 is a graph showing results obtained in a one-step reaction process where the reverse transcriptase and polymerase are included in a single reaction.

FIG. 9 is a graph showing results obtained in a two-step reaction process where the reverse transcriptase reaction is carried out at room temp or 56° C. and the cDNA is transferred to a second tube where the amplification reaction is carried out at 56° C.

FIG. 10 is a graph showing RT at 56° C., DNAble at 56° C. on 1×10e⁴ copies.

FIGS. 11A and 11B show the detection of a target RNA in a sample containing total cellular RNA. FIG. 11A is a graph showing detection of RPPH1 (RNase P RNA Component) in a 2-step reaction. FIG. 11B is a graph showing detection of RPPH1 in a 1-step reaction.

FIG. 12 is a graph showing the detection of Zaire Ebola in a crude blood preparation in a 1-step reaction.

FIG. 13 is a graph depicting that the Zaire Ebola assay specifically detects Zaire Ebola Mayinga but not Sudan Ebola Boniface.

FIG. 14 are graphs depicting instrument comparison for the detection of various dilutions of Zaire Ebolavirus Mayinga.

FIGS. 15A-15D provide example amplification curves (10 technical replicates each) representing serially diluted one-step RNABLE® Zaire Ebolavirus Mayinga RNA that depict the limit of detection of the Ebola virus assay. FIG. 15A is a graph depicting detection in samples containing 100 copies of Ebola virus target RNA. FIG. 15B is a graph depicting detection in samples containing 50 copies of Ebola virus target RNA. FIG. 15C is a graph depicting detection in samples containing 25 copies of Ebola virus target RNA. FIG. 15D is a graph depicting detection in samples containing 12 copies of Ebola virus target RNA.

FIGS. 16A and 16B show detection of human immunodeficiency virus (HIV) in a one-step assay. FIG. 16A is an amplicon map showing sequences used in the design of assay primers and probes (SEQ ID NOs: 67-68, respectively, in order of appearance). Population sequence variations in forward and reverse primers are indicated (SEQ ID NOs: 6-9, 43 and 10, respectively, in order of appearance). External primer sequence is specific to HIV subtype C (for the purified RNA sample used). FIG. 16B shows real-time target specific amplification of HIV in a one-step assay. Cp values are shown across 5 technical replicates for each copy number (10 μL reactions). Copies of purified HIV RNA (subtype C) (SeraCare) are indicated, as quantified by COBAS TaqMan HIV-1 v2.0 test FIGS. 17A and 17B show detection of dengue virus type 4 (DENV-4) in a one-step assay. FIG. 17A is an amplicon map showing sequences used in the design of assay primers and probes (SEQ ID NOs: 69-70, respectively, in order of appearance). Population sequence variations in reverse primers are indicated (SEQ ID NOs: 11-13, 44 and 14, respectively, in order of appearance). FIG. 17B shows real-time target specific amplification of DENV-4 in a one-step assay. Cp values are shown across 4 technical replicates (10 μL reactions). Isolated total RNA (20 pg) from cell culture included both viral and host cell RNA and total copy number of viral RNA was unknown.

FIGS. 18A and 18B show detection of influenza B in a one-step assay. FIG. 18A is an amplicon map showing sequences used in the design of assay primers and probes (SEQ ID NOs: 71-73, respectively, in order of appearance). Population sequence variations in forward and reverse primers and external primers are indicated (SEQ ID NOs: 15-20, 45, 46 and 21, respectively, in order of appearance). FIG. 18B shows real-time target specific amplification of influenza B in a one-step assay. Cp values are shown across 4 technical replicates (10 μL reactions). Isolated total RNA (20 pg) from cell culture included both viral and host cell RNA and total copy number of viral RNA was unknown. Samples 1 and 2 are different viral isolates.

FIGS. 19A and 19B show detection of Bovine Viral Diarrhea Virus Genotype 1 (BVDV1) in a one-step assay. FIG. 19A is an amplicon map showing sequences used in the design of assay primers and probes (SEQ ID NOs: 74-75, respectively, in order of appearance). Population sequence variations in forward primers are indicated (SEQ ID NOs: 22-24, 47 and 25, respectively, in order of appearance). FIG. 19B shows real-time target specific amplification of Bovine Viral Diarrhea Virus Genotype 1 (BVDV1) in a one-step assay. Technical replicates (10 μL reactions) are shown. Isolated total RNA (20 pg) from cell culture included both viral and host cell RNA and total copy number of viral RNA was unknown. Cell culture crude lysate was used undiluted and at 1:100 dilution.

### DETAILED DESCRIPTION OF THE INVENTION

The present invention provides methods for rapidly identifying an RNA viral infection (e.g., Ebola virus) using an isothermal nucleic acid amplification reaction that can be carried out on extracted RNA in the context of a crude biological sample.

Ebola is clinically difficult to diagnose and to distinguish. A rapid and reliable laboratory diagnosis is required in suspected cases of Ebola. The present invention provides such an assay. The invention is based, at least in part, on the discovery that an Ebola viral polynucleotide (e.g., RNA) can be detected in a one-step or two-step real-time reverse transcription-isothermal amplification assay for an Ebola viral polynucleotide.

### Ebola Virus

The Ebola viruses are filamentous viruses with a negative-sense RNA genome. Virions are cylindrical/tubular containing a viral envelope, matrix, and nucleocapsid components, approximately 80 nm in diameter and 800-1000 nm in length. Ebola is classified as a biosafety level 4 agent. The period of incubation for the Ebola virus hemorrhagic fever is usually 5-18 days, but may extend from 2-21 days depending on the viral strain contracted and the condition of the infected individual. The Ebola virus acts quickly. Initial symptoms of Ebola resemble symptoms of malaria, influenza, or various bacterial infections. Therefore, days or weeks may pass before Ebola is diagnosed. Secondary symptoms include diarrhea, red eyes, vomiting blood, bleeding from the nose, mouth or rectum, and even bleeding in the brain. About 50%-90% of those infected with the virus go on to systemic multi-organ failure and death.

### Patient Diagnosis and Monitoring

The condition of a patient as having or not having Ebola can be diagnosed by detecting an Ebola viral polynucleotide in a biological sample and correlating this detection with the existence of an Ebola infection. In one embodiment, a disease state of a patient having Ebola virus can be detected using the methods and compositions of the invention to detect Ebola virus in a biological sample of the patient. Exemplary biological samples include body fluids (e.g. saliva, sweat, tears, fluids accumulating in a bodily cavity, urine, ejaculate, vaginal secretion, cerebrospinal fluid, lymph, feces, sputum, decomposition fluid, vomit, sweat, breast milk, blood, serum, and plasma), tissue extracts, culture media (e.g., a liquid in which a cell, such as a pathogen cell, has been grown), or environmental samples obtained, for example, from a material that may be contaminated with a biological fluid of a subject.

In one embodiment, the invention provides a method of amplifying a target polynucleotide in a reverse transcriptase and nicking amplification reaction involving:
(a) contacting a target RNA molecule (e.g., Ebola virus genome) with a reverse transcriptase (RT) primer in the presence of a reverse transcriptase and dNTPs under conditions permissive for cDNA synthesis, thereby generating a cDNA; and
(b) contacting the cDNA with forward and reverse primers carrying at least one nicking enzyme recognition sequence within their respective 5′-terminal regions which specifically bind the cDNA with their respective 3′-terminal regions in the presence of a nicking enzyme, dNTPs, and a strand-displacement polymerase under conditions permissive for the isothermal amplification of the cDNA, thereby generating amplicons.

In one particular embodiment, the invention provides a method of detecting an Ebola virus in a biological sample involving:
(a) contacting a sample with an agent capable of extracting an Ebola RNA present in the sample (e.g., SDS, sodium lauryl sulfate, guanidium isothio-cyanate, guanidium hydrochloride) and an agent capable of stabilizing Ebola RNA against degradation (e.g., SDS, RNAase inhibitors, antibodies against RNAse, competitive RNAse inhibitor, or an agent capable of reversibly chemically modifying RNA in situ (e.g., acetic anhydride);
(b) contacting the Ebola RNA with a RT primer in the presence of a reverse transcriptase and dNTPs under conditions permissive for cDNA synthesis, thereby generating a cDNA copy of the Ebola RNA;
(c) contacting the Ebola cDNA with forward and reverse primers carrying at least one nicking enzyme recognition sequence within their respective 5′-terminal regions which specifically binds the Ebola cDNA with their respective 3′-terminal regions in the presence of a nicking enzyme, dNTPs, and a strand-displacement polymerase under conditions permissive for the isothermal amplification of the cDNA, thereby generating amplicons; and
(d) detecting the amplicons, wherein the presence of an Ebola virus amplicon detects an Ebola virus infection in the sample and failure to detect the amplicon indicates the absence of an Ebola virus infection.

In one embodiment, the methods of the invention described herein provide results in no longer than 5, 7, 10, 15, 20, 25 or 30 minutes. Advantageously, the methods of the invention can be applied to RNAs extracted—but not purified or isolated from—a crude biological sample (e.g., saliva, sweat, tears, fluids accumulating in a bodily cavity, urine, ejaculate, vaginal secretion, cerebrospinal fluid, lymph, feces, sputum, decomposition fluid, vomit, sweat, breast milk, blood, serum, and plasma). Because the test is carried out on-site (e.g., in a hospital, clinic, physician's office, urgent care center, home, community center, airport, ship (e.g., cruise ship or other vessel used for transporting humans or animals), train or train station, or point of entry into a nation (e.g., border crossing). Advantageously, in one embodiment, the testing is carried out in a portable battery powered device (e.g., Amplifire).

In one embodiment, the RNA is extracted from the biological sample using a chaotropic salt (e.g., GITC, GHCL) or a detergent (e.g., SDS, Tween and triton). If desired, an RNAse inhibitor is added before, during, or after the extraction step.

In another embodiment, the reverse transcriptase enzyme and the strand-displacement DNA polymerase are one and the same.

In another embodiment, no separate primer is required or the RT primer same as forward and/or reverse primers.

In another embodiment, the invention provides for detection of an EBOV or other viral amplicon using a Dual FRET molecular beacon for mRNA detection (e.g., as described by Santagelo, Nucleic Acids Res. 2004; 32(6): e57), turbidity release of pyrophosphate from DNTPs and precipitation with magnesium or calcium.

In another embodiment, the invention provides for detection of an Ebola viral amplicon using a lateral flow device where the Ebola virus amplicon comprises one member of a pair of binding partners (e.g., biotin and streptavidin) and the lateral flow device comprises the other member of the pair, and provides a means of detection (e.g., colorimetric) for the amplicon.

Reverse transcriptases used in the methods of the invention include, but are not limited to, a Maloney murine leukemia virus reverse transcriptase enzyme (MMLV RT) and derivatives or variants thereof comprising a mutation relative to wild-type MMLV RT; avian myeloblastosis virus (AMV RT) and derivatives or variants thereof comprising a mutation relative to that render them thermostable, Rous sarcoma virus (RSV) RT (e.g., Omniscript, Qiagen) and derivatives or variants thereof, and a pyroreverse transcriptase (e.g., Pyroscript luceigen) and derivatives or variants thereof, an RT described in U.S. Pat. No. 7,094,539, which is incorporated herein by reference in its entirety, or a commercially available High-fidelity Thermostable Reverse Transcriptase for RT PCR and Transcriptome analysis (e.g., Lucigen).

In one embodiment, at 56 degrees the primer and RNA or amplicon forms a stable complex. In one embodiment, more than 50% of primer sequence must be complementary to the target nucleic acid molecule. In one embodiment, the rT primer is about 18 bases in length. In one embodiment, the reverse transcriptase (RT) primer is a random primer (e.g., in each sequence position any one of four bases is possible, any of these primers hybridize with the target). In one embodiment, the rT primer is a hexamer, heptamer, octamer, or nonamer.

In one embodiment, the RT is derived from Geobacillus stearothermophilus, is M-MLV RT (i.e. Superscript/LifeTech, Maxima/Thermo-Fisher) and/or mutants/derivatives thereof, AMV RT (i.e. Thermoscript/LifeTech) and/or mutants/derivatives thereof, RSV RT (i.e. OmniScript/Qiagen) and/or mutants/derivatives thereof.

Methods of the invention provide a high degree of sensitivity. In one embodiment, EBOV is detected at 1×10³, 1×10⁴, 1×10⁵, 1×10⁶, 1×10⁷, or 1×10⁹ copies of EBOV RNA per ml in blood. In another embodiment, the invention provides for the detection of between about 1-10 (e.g, 1, 2, 3, 4, 5, 6, 7, 8, 9, 10) copies of RNA per reaction.

EBOV (or other virus) is detected by obtaining a sample (e.g., biological sample) from a subject having or suspected of having an EBOV infection or by obtaining an environmental sample from a home, hospital room, means of transportation that is or is suspected of being contaminated with an Ebola virus or EBOV-containing biological fluid. In one embodiment, a biological sample is obtained by obtaining a blood sample, mucous sample, feces, or by swabbing an affected tissue. Swabs can be taken from the nose, throat, eyes, or other mucosal membrane. At necropsy, samples can be collected from blood or tissues of the deceased.

Advantageously, the diagnostic methods of the invention are suitable for use in virtually any setting. EBOV is endemic in much of west Africa including Liberia, Nigeria, Guinea, and Sierra Leone. Many areas within west Africa lack access to basic medical facilities and diagnostic laboratories. The present invention can be used in a battery powered hand held device that is well-suited to testing of biological samples in areas where access to electricity is non-existent. Moreover, the present methods are simple enough that they can easily be carried out by health workers who have limited training in the use of diagnostic technologies.

The present invention provides methods for rapidly identifying an EBOV or other viral infection using an isothermal nucleic acid amplification reaction that can be carried out on extracted RNA in the context of a crude biological sample.

Early in the disease process, only low levels of virus are present in a biological sample of the subject, such as a blood sample. If desired, the virions present in the sample are enriched using methods known in the art, for example, by precipitating the virions from the sample by adding PEG and NaCl then filtering virions out of the sample using a nanopore filter, thereby providing for early detection of a viral polynucleotide.

The disease state or treatment of a subject that may have been exposed to Ebola virus (or other virus) can be monitored using the methods and compositions of the invention. In one embodiment, the detection of an Ebola virus polynucleotide (or other virus polynucleotide) is present in a bodily fluid, such as saliva, sweat, tears, fluids accumulating in a bodily cavity, urine, ejaculate, vaginal secretion, cerebrospinal fluid, lymph, feces, sputum, decomposition fluid, vomit, sweat, breast milk, blood, serum, and plasma, is monitored. Such monitoring may be useful, for example, in diagnosing the subject as having Ebola (or other virus), or determining the efficacy of a particular drug in a subject or in assessing disease progression.

### Nucleic Acid Amplification Methods

Nucleic acid amplification technologies have provided a means of understanding complex biological processes, detection, identification, and quantification of pathogenic organisms, such as EBOV or other RNA viruses. The present invention provides for the detection of an EBOV negative-sense RNA genome in a biological sample by using reverse transcriptase to synthesize an EBOV DNA molecule from the RNA genome and then amplifying the DNA in an isothermal nicking amplification reaction.

The polymerase chain reaction (PCR) is a common thermal cycling dependent nucleic acid amplification technology used to amplify DNA consisting of cycles of repeated heating and cooling of the reaction for DNA melting and enzymatic replication of the DNA using a DNA polymerase. Real-Time quantitative PCR (qPCR) is a technique used to quantify the number of copies of a given nucleic acid sequence in a biological sample. Currently, qPCR utilizes the detection of reaction products in real-time throughout the reaction and compares the amplification profile to the amplification of controls which contain a known quantity of nucleic acids at the beginning of each reaction (or a known relative ratio of nucleic acids to the unknown tested nucleic acid). The results of the controls are used to construct standard curves, typically based on the logarithmic portion of the standard reaction amplification curves. These values are used to interpolate the quantity of the unknowns based on where their amplification curves compared to the standard control quantities.

In addition to PCR, non-thermal cycling dependent amplification systems or isothermal nucleic acid amplification technologies exist including, without limitation: Nicking Amplification Reaction, Rolling Circle Amplification (RCA), Helicase-Dependent Amplification (HDA), Loop-Mediated Amplification (LAMP), Strand Displacement Amplification (SDA), Transcription-Mediated Amplification (TMA), Self-Sustained Sequence Replication (3SR), Nucleic Acid Sequence Based Amplification (NASBA), Single Primer Isothermal Amplification (SPIA), Q-P Replicase System, and Recombinase Polymerase Amplification (RPA).

Isothermal nicking amplification reactions have similarities to PCR thermocycling. Like PCR, nicking amplification reactions employ oligonucleotide sequences which are complementary to a target sequences referred to as primers. In addition, nicking amplification reactions of target sequences results in a logarithmic increase in the target sequence, just as it does in standard PCR. Unlike standard PCR, the nicking amplification reactions progress isothermally. In standard PCR, the temperature is increased to allow the two strands of DNA to separate. In nicking amplification reactions, the target nucleic acid sequence is nicked at specific nicking sites present in a test sample. The polymerase infiltrates the nick site and begins complementary strand synthesis of the nicked target nucleotide sequence (the added exogenous DNA) along with displacement of the existing complimentary DNA strand. The strand displacement replication process obviates the need for increased temperature. At this point, primer molecules anneal to the displaced complementary sequence from the added exogenous DNA. The polymerase now extends from the 3′ end of the template, creating a complementary strand to the previously displaced strand. The second oligonucleotide primer then anneals to the newly synthesized complementary strand and extends making a duplex of DNA which includes the nicking enzyme recognition sequence. This strand is then liable to be nicked with subsequent strand displacement extension by the polymerase, which leads to the production of a duplex of DNA which has nick sites on either side of the original target DNA. Once this is synthesized, the molecule continues to be amplified exponentially through replication of the displaced strands with new template molecules. In addition, amplification also proceeds linearly from each product molecule through the repeated action of the nick translation synthesis at the template introduced nick sites. The result is a very rapid increase in target signal amplification; much more rapid than PCR thermocycling, with amplification results in less than ten minutes.

### Nicking Amplification Assays

The invention provides for the detection of EBOV target nucleic acid molecules amplified in an isothermal nicking amplification assay.

Polymerases useful in the methods described herein are capable of catalyzing the incorporation of nucleotides to extend a 3′ hydroxyl terminus of an oligonucleotide (e.g., a primer) bound to a target nucleic acid molecule and/or a 3′ hydroxyl terminus at a nick site in a double-stranded DNA molecule in conjunction with strand displacement activity. Such polymerases also lack or have substantially reduced 5′-3′ exonuclease activity and may include those that are thermophilic. DNA polymerases useful in methods involving primers having 2′-modified nucleotides in the primer region comprising the six 3′-terminal nucleotides include derivatives and variants of the DNA polymerase I isolated from Bacillus stearothermophilus, also classified as Geobacillus stearothermophilus, and from closely related bacterial strains, isolates and species comprising the genus Geobacillus, which lack or have substantially reduced 5′-3′ exonuclease activity and have strand-displacement activity. Exemplary polymerases include, but are not limited to, the large fragments of Bst DNA polymerase I, Gst DNA polymerase I, and Gka DNA polymerase I.

A nicking agent useful in methods described herein is a chemical entity capable of recognizing and binding to a specific structure in double stranded nucleic acid molecules and breaking a phosphodiester bond between adjoining nucleotides on the top strand with a substantially higher rate than breaking the phosphodiester bond between adjoining nucleotides on the bottom strand upon binding to its recognized specific structure, thereby creating a free 3′-hydroxyl group on the terminal nucleotide preceding the nick site that can be extended by a 5′-3′-exonuclease deficient strand displacement polymerase. In a preferred embodiment of the methods disclosed herein, the top strand phosphodiester bond cleavage rate of the “nicking agent” approaches 100%, while the cleavage rate of the bottom strand phosphodiester bond approaches 0%. Nicking agents useful in methods described herein, can either be enzymes, i.e. self-regenerating catalysts turning over multiple substrate molecules, or non-regenerating catalysts turning over just a single substrate molecule at an equimolar ratio fashion.

A nicking enzyme binds double-stranded DNA and cleaves one strand of a double-stranded duplex. In the methods of the invention, the nicking enzyme cleaves the top stand (the strand comprising the 5′-3′ sequence of the nicking agent recognition site). In a particular embodiment of the invention disclosed herein, the nicking enzyme cleaves the top strand only and 3′ downstream of the recognition site. In exemplary embodiments, the reaction comprises the use of a nicking enzyme that cleaves or nicks downstream of the binding site such that the product sequence does not contain the nicking site. Using an enzyme that cleaves downstream of the binding site allows the polymerase to more easily extend without having to displace the nicking enzyme. Ideally, the nicking enzyme is functional under the same reaction conditions as the polymerase. Exemplary nicking enzymes include, but are not limited to, N.Bst9I, N.BstSFI, Nb.BbvCI(NFB), Nb.Bpu10I(Fermantas), Nb.BsmI(NFB), Nb.BsrDI(NFB), Nb.BtsI(NFB), Nt.AlwI(NFB), Nt.BbvCI(NFB), Nt.Bpu10I(Fermentas), Nt.BsmAI, Nt.BspD6I, Nt.BspQI(NFB), Nt.BstNBI(NFB), and Nt.CviPII(NFB). Sequences of nicking enzyme recognition sites are provided at Table 1.

TABLE 1
Nicking enzyme recognition sequences

| | |
|---|---|
| N.Bst9I | 5′-GAGTCNNNNN↓NN-3′ |
| | \|\|\|\|\|\|\|\|\|\| \|\| |
| | 3′-CTCAGNNNNN•NN-5′ |
| N.BstSEI | 5′-GAGTCNNNNN↓NN-3′ |
| | \|\|\|\|\|\|\|\|\|\| \|\| |
| | 3′-CTCAGNNNNN•NN-5′ |
| Nb.BbvCI(NEB) | 5'-CCTCA•GC-3' |
| | \|\|\|\|\| \|\| |
| | 3′-GGAGT↑CG-5′ |
| Nb.Bpu10I(Fermantas) | 5′-CCTNA•GC-3′ |
| | \|\|\|\|\| \|\| |
| | 3′-GGANT↑CG-5′ |
| Nb.BsmI(NEB) | 5′-GAATG•CN-3′ |
| | \|\|\|\|\| \|\| |
| | 3′-CTTAC↑GN-5′ |
| Nb.BsrDI(NEB) | 5′-GCAATG•NN-3′ |
| | \|\|\|\|\| \|\| |
| | 3′-CGTTAC↑NN-5′ |
| Nb.BtsI(NEB) | 5′-GCAGTG•NN-3′ |
| | \|\|\|\|\| \|\| |
| | 3′-CGTCAC↑NN-5′ |
| Nt.AlwI(NEB) | 5′-GGATCNNNN↓N-3′ |
| | \|\|\|\|\|\|\|\|\| \| |
| | 3′-CCTAGNNNN•N-5′ |
| Nt.BbvCI(NEB) | 5′-CC↓TCAGC-3′ |
| | \|\| \|\|\|\|\| |
| | 3′-GG•AGTCG-5′ |
| Nt.Bpu10I(Fermentas) | 5'-CC↓TNAGC-3′ |
| | \|\| \|\|\|\|\| |
| | 3′-GG•ANTCG-5′ |
| Nt.BsmAI | 5′-GTCTCN↓N-3′ |
| | \|\|\|\|\|\| \| |
| | 3′-CAGAGN•N-5′ |
| Nt.BspD6I | 5′-GAGTCNNNN↓N-3′ |
| | \|\|\|\|\|\|\|\|\| \| |
| | 3′-CTCAGNNNN•N-5′ |
| Nt.BspQI(NEB) | 5′-GCTCTTCN↓-3′ |
| | \|\|\|\|\|\|\|\| |
| | 3′-CGAGAAGN-5′ |
| Nt.BstNBI(NEB) | 5′-GAGTCNNNN↓N-3′ |
| | \|\|\|\|\|\|\|\|\| \| |
| | 3′-CTCAGNNNN•N-5′ |
| Nt.CviPII(NEB) | 5′-↓CCD-3′ |
| | \|\|\| |
| | 3′- GGH-5′ |

Nicking enzymes also include engineered nicking enzymes created by modifying the cleavage activity of restriction endonucleases (NEB expressions July 2006, vol 1.2). when restriction endonucleases bind to their recognition sequences in DNA, two catalytic sites within each enzyme for hydrolyzing each strand drive two independent hydrolytic reactions which proceed in parallel. Altered restriction enzymes can be engineered that hydrolyze only one strand of the duplex, to produce DNA molecules that are “nicked” (3′-hydroxyl, 5′-phosphate), rather than cleaved. Nicking enzymes may also include modified CRISPR/Cas proteins, Transcription activator-like effector nucleases (TALENs), and Zinc-finger nucleases having nickase activity.

A nicking amplification reaction typically comprises nucleotides, such as, for example, dideoxyribonucleoside triphosphates (dNTPs). The reaction may also be carried out in the presence of dNTPs that comprise a detectable moiety including but not limited to a radiolabel (e.g., ³²P, ³³P, ¹²⁵I, ³⁵S) an enzyme (e.g., alkaline phosphatase), a fluorescent label (e.g., fluorescein isothiocyanate (FITC)), biotin, avidin, digoxigenin, antigens, haptens, or fluorochromes. The reaction further comprises certain salts and buffers that provide for the activity of the nicking enzyme and polymerase.

Advantageously, the nicking amplification reaction is carried out under substantially isothermal conditions where the temperature of the reaction is more or less constant during the course of the amplification reaction. Because the temperature does not need to be cycled between an upper temperature and a lower temperature, the nicking amplification reaction can be carried out under conditions where it would be difficult to carry out conventional PCR. Typically, the reaction is carried out at about between 35 C and 90 C (e.g., about 35, 37, 42, 55, 60, 65, 70, 75, 80, or 85° C.). Advantageously, it is not essential that the temperature be maintained with a great degree of precision. Some variability in temperature is acceptable.

Sets of primers for amplification reactions are selected as having ΔΔG's≤−15, −16, 17, −18, −19, −20, −25, −30 kcal/mole or more. The performance characteristics of amplification reactions may be altered by increasing the concentration of one or more oligonucleotides (e.g., one or more primers and/or probes) and/or their ratios. High concentrations of primers also favor primer-dimer formation. In various embodiments, concentration of a primers is 100, 200, 300, 400, 500, 600, 700, 800, 900, 1000 nM or more. Melt temperature (Tm) and reaction rate modifiers may also be used to lower the melting temperature of the oligonucleotides, such as (but not limited to) ethylene glycol and glycerol. In addition, DNA polymerase reaction rate modifiers (such as dNTP and magnesium concentration) may be used to alter the reaction rate to lead to a greater quantification precision. In particular embodiments, the 5′ tail sequences of the forward and reverse primers have the same nucleic acid sequence.

This invention provides methods of monitoring a nicking amplification reaction in real time, including for example utilizing the amplification strategy as described herein. In one embodiment, quantitative nucleic acid amplification utilizes target nucleic acids amplification alongside a control amplification of known quantity. The amount of target nucleic acid can be calculated as an absolute quantification or a relative quantification (semi-quantitative) based on the source of the control (exogenous or endogenous control).

Quantification of the unknown nucleotide sequence can be achieved either through comparison of logarithmic threshold amplification of the unknown to a series of known target sequences in either a separate set of reactions or in the same reaction; or as an internal endogenous or exogenous co-amplification product, which produces a threshold value, indicative of either a positive result (if the unknown exceeds the threshold) or negative result (if the unknown does not exceed the threshold).

The invention also provides a method of designing a nicking agent-dependent isothermal strand-displacement amplification assay without experimental screening of a multitude of combinations of candidate forward primers and/or candidate reverse primers. A 35 to 70 bp long region within the target sequence is identified having a 12 to 20 bp sequence in the central portion with a Tm≥the assay temperature (e.g., ˜55° C.). Adjacent sequences 12 bp to 20 bp long immediately downstream and upstream of the 15 to 20 bp long central region are identified, according to the above criteria. The Tm of the chosen double stranded downstream and upstream adjacent sequences deviate from each other by less than ±3° C. A target-specific pair of forward and reverse primers are created by attaching a 5′-tail region for a stable dimer-forming primer to the 5′-terminus of the 12-20 base upstream adjacent sequence and to the 5′-terminus of the complementary strand of the 12-20 base downstream adjacent sequence. When combining the forward primer, reverse primer, and a probe, the primer driving the synthesis of the strand complementary to the probe is in excess over the other primer at a molar ratio of about 1.1:1 to 10:1. The combined concentration of a primer in the assay is no higher than 1000 nM. The assay design method can also be used to convert a pre-validated PCR assay for an amplicon ≤70 bp to a nicking agent-dependent isothermal strand-displacement amplification assay.

### Primer Design

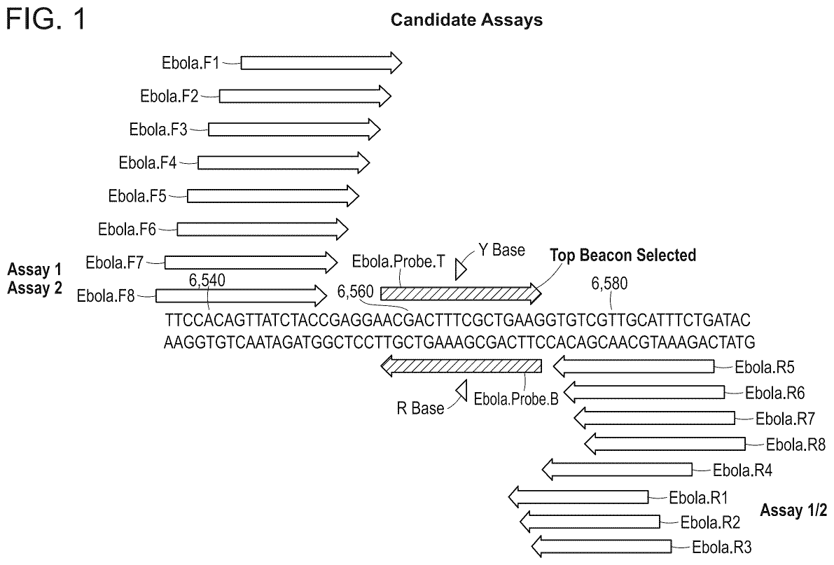

Conventional methods for primer design have focused on primer melting temperature, primer annealing temperature, GC (guaninine and cytosine) content, primer length, and minimizing interactions of the primer with all but the target nucleic acid (see e.g., premierbiosoft.com). Contrary to these methods, it has been found that primers that form stable primer/dimers, expressed in terms of free energy of formation (ΔG), function predictably in nucleic acid amplification reactions. While Free Energy (ΔG) and Melting Temperature (Tm) share primary components Enthalpy (AH) and Entropy (ΔS), ΔG and Tm values are derived differently and have no correlative relationship, and the only way to relate a given ΔG with a given Tm value is to explicitly know the value of ΔH and ΔS from which they are derived (Manthey, “mFold, Delta G, and Melting Temperature” ©2005 and 2011 Integrated DNA Technologies). FIGS. 1-11 relate to the design of optimal primers.

The free energy of formation (ΔG) for intermolecular primer structures may be calculated using formulas known in the art. A number of programs are available for determining the formation of various intramolecular and intermolecular primer structures and calculating their ΔG's, including for example mfold and UNAfold prediction algorithms (see e.g., Markham and Zuker. UNAFold: Software for Nucleic Acid Folding and Hybridization. Bioinformatics: Volume 2, Chapter 1, pp 3-31, Humana Press Inc., 2008; Zuker et al. Algorithms and Thermodynamics for RNA Secondary Structure Prediction: A Practical Guide In RNA Biochemistry and Biotechnology, 11-43, NATO ASI Series, Kluwer Academic Publishers, 1999; M. Zuker. Prediction of RNA Secondary Structure by Energy Minimization. Methods in Molecular Biology, 267-294, 1994; Jaeger et al. Predicting Optimal and Suboptimal Secondary Structure for RNA. In Molecular Evolution: Computer Analysis of Protein and Nucleic Acid Sequences, Methods in Enzymology 183, 281-306, 1990; Zuker. On Finding All Suboptimal Foldings of an RNA Molecule. Science 244, 48-52, 1989). OligoAnalyzer 3.1 is one such implementation of mfold for primer design (idtdna.com). For example, with reference to OligoAnalyzer 3.1, ΔG calculations may be performed using the following parameters: Target Type: DNA; Oligo Concentration 0.25 μM; Na⁺ Concentration: 60 mM; Mg⁺⁺ Concentration: 15 mM; and dNTPs Concentration: 0.3 mM.

### 3′ Recognition Region

The invention provides a primer having a 3′ recognition sequence whose primer-target formation is stable (ΔG≤about −20 kcal/mol or more) and has the potential to enhance nucleic acid amplification reaction performance. The 3′ recognition region specifically binds to the nucleic acid molecule, for example a complementary sequence of the nucleic acid molecule. In certain embodiments, the 3′ recognition region has a sequence that is complementary to 12, 13, 14, 15, 16, 17, 18, 19, or 20 bases or more of a nucleic acid sequence. In particular embodiments, the 3′ recognition region comprises one or more inosine bases. In specific embodiments, the 3′ recognition region comprises no more than 2/12 inosines. In various embodiments, the primer-target melting temperature is equal to or greater than 8° or 6° C. below the reaction or extension temperature of the assay (Tm≥assay temperature−8°). In particular embodiments, the 3′ recognition sequence comprises 12-20, 12-17, or 12-14 bases. In particular embodiments, the primer-target formation is more stable than self dimer formation (e.g., ΔΔG about −15, −16, −17, −18, −19, −20 kcal/mol or more). Preferably, the 3′ recognition sequence does not contain self-complementary sequences, short inverted repeats (e.g., >4 bases/repeat), or sequences that otherwise promote intramolecular interactions, which have the potential to interfere with primer-target annealing.

In one embodiment, a primer is designed having a Tm of 56° C. with 4 sequence specific bases at the end of the primer that may not contribute to annealing. In one embodiment, the primer is a 16, 17, 18, 19, 20 or 21-mer.

In particular, a primer of the invention having a 3′ recognition sequence is useful in nicking amplification assays. Additionally, the EBOV or other viral target specific 3′ recognition region comprises one or more 2′ modified nucleotides (e.g., 2′-O-methyl, 2′-methoxyethoxy, 2′-fluoro, 2′-alkyl, 2′-allyl, 2′-O-[2-(methylamino)-2-oxoethyl], 2′-hydroxyl (RNA), 4′-thio, 4′-CH₂—O-2′-bridge, 4′-(CH₂) 2-O-2′-bridge, and 2′-O—(N-methylcarbamate)). Without being bound to theory, it is hypothesized that incorporating one or more 2′ modified nucleotides in the recognition regions reduces or eliminates intermolecular and/or intramolecular interactions of primers/templates (e.g., primer-dimer formation), and, thereby, reduces or eliminates the background signal in isothermal amplification. The 2′ modified nucleotide preferably has a base that base pairs with the target sequence. In particular embodiments, two or more 2′ modified nucleotides (e.g., 2, 3, 4, 5 or more 2′ modified nucleotides) in the EBOV or other viral target specific recognition region are contiguous (e.g., a block of modified nucleotides). In some embodiments, the block of 2′ modified nucleotides is positioned at the 3′ end of the target specific recognition region. In other embodiments, the block of 2′ modified nucleotides is positioned at the 5′ end of the EBOV or other viral target specific recognition region. When the block of 2′ modified nucleotides is positioned at the 5′ end of the target specific recognition region, the 2′ modified nucleotides may be separated from the nick site by one or more non-modified nucleotides (e.g., 2, 3, 4, 5 or more 2′ unmodified nucleotides). Applicants have found that positioning of one or more 2′ modified nucleotides or of a block of 2′ modified nucleotides alters the kinetics of amplification. When the one or more 2′ modified nucleotides or block of 2′ modified nucleotides are positioned at or near the 5′ end of the recognition region or proximal to the nick site, real-time amplification reactions showed decreased time to detection. Additionally, the signal curve is contracted and the slope of the curve shifted.

In a related embodiment, ratios of a primer having one or more 2′ modified nucleotides can be used to alter the time-to-detection and/or the efficiency of the reaction for the ‘tuning’ of reactions, resulting in a predictable control over reaction kinetics. Increasing the ratio of primer having one or more 2′ modified nucleotides at the 3′ end of the recognition sequence to primer having one or more 2′ modified nucleotides at the 5′ end of the recognition sequence contracted the signal curve and shifted the slope of the curve. It is advantageous to be able to “tune” a reaction providing a means to manipulate both the time-to-detection as well as the efficiency of the reaction. Relative quantification using an internal control requires that two important conditions be met. First, it is beneficial to be able to modify a reaction's time-to-detection creating a non-competitive reaction condition. Thus, by affecting the control reaction to be detectable at a later time-point (relative to the target of interest) the control reaction does not out-compete the specific target of interest even when the target of interest is in low initial abundance. Second, to ensure a true relative abundance calculation, it is required that the control and specific target reactions have matched efficiencies. By controlling the efficiency of each reaction using a “tuning” condition enables reactions to be matched allowing for satisfactory relative quantification calculations. Tuning the reactions can be used to match efficiencies of target nucleic acid amplification and reference nucleic amplification (e.g., internal standard) in quantitative PCR (qPCR). Additionally, amplification curves of the target nucleic acid and the internal standard may be altered so time of detection of their amplification products are separated, while providing the same efficiency for target nucleic acid amplification and internal standard amplification. Through the use of specific combinations and ratios of oligonucleotide structures within a reaction it is possible to create conditions which enable tuned reaction performance.

### 5′ Tail Dimerization Region

The invention provides a primer having a 5′ tail region capable of self-dimerization that enhances EBOV or other viral nucleic acid amplification reaction performance. Without being bound to theory, in a nucleic acid amplification reaction the primer anneals to the target nucleic acid as a primer-dimer. For example, nicking amplification primers have a nicking agent recognition site present at the 5′ end that is unrelated to the binding specificity of the primer for the target recognition sequence. Non-specific background products from non-specific primer interactions have the potential to sequester reaction components that would otherwise have been utilized for the amplification of the specific product. In various embodiments, homodimer formation is stable (e.g., ΔG about −30, −35, −40, −45, −50, −55, −60 kcal/mol or more). In various embodiments, the homodimer has a melting temperature higher than the extension reaction temperature. In particular embodiments, the 5′ tail region has a sequence that is a palindrome. In further embodiments, the 5′ tail region is at least 12 bases (e.g., 12, 13, 14, 15, 16, 17, 18, 19, 20, 21, 22, 23, 24 bases) in length. In additional embodiments, the 5′ tail region has a GC content of 80-90%. In certain embodiments, homodimer formation is more stable than formation of other less stable primer dimer conformations formation (e.g., ΔΔG about −12, −13, −14, −15, −16, −17, −18, −19, −20, −25, −30, −35, −40 kcal/mol or more).

In particular, a primer of the invention having a 5′ tail sequence is useful in nicking amplification reactions. For use in nicking amplification reactions, the 5′ tail region comprises one or more nicking agent recognition sites and the 5′ tail region has a symmetrically inverted sequence. In particular embodiments, the 5′ tail region contains an even number of nucleotides (e.g., 22, 24 nucleotides). The nick site is designed to be positioned between the nucleotide at the 3′ end of the 5′ tail region and the nucleotide at the 5′ end of the 3′ recognition region. Without being bound to theory, the nicking enzyme does not cleave at the nick site when the 3′ recognition is single-stranded. However, cleavage at the nick site occurs when the 3′ recognition region is double stranded (e.g., when the primer is incorporated into a double-stranded target nucleic acid molecule during the course of the nucleic acid amplification reaction).

In various embodiments, the 5′ tail sequence comprises from 5′ to 3′ an inverted nicking enzyme recognition sequence that is operatively linked to a palindromic sequence (or self-complementary sequence) that is operatively linked to a nicking enzyme recognition sequence. In certain embodiments, the spacer region is an even number of nucleotides (e.g., 2, 4, 6, etc.). Exemplary 5′ tails based on the Nt.BstNBI nicking enzyme recognition sequence (5′-GAGTC-3′) having a 2, 4, and 6 nucleotide spacers comprise a nucleic acid sequences according to the formula below:

(SEQ ID NO: 31)
5′-GACTCN₁N_{1′}GAGTC-3′
(SEQ ID NO: 32)
5′-GACTCN₂N₁N_{1′}N_{2′}GAGTC-3′
(SEQ ID NO: 33)
5′-GACTCN₃N₂N₁N_{1′}N_{2′}N_{3′}GAGTC-3′

where “N” is any nucleotide (e.g., having an adenine (A), thymine (T), cytosine (C), or guanine (G) nucleobase), and Ni is complementary to N_{1′}, N₂ is complementary to N_{2′}, and N₃ is complementary to N_{3′}, etc.

Exemplary 5′ tail region sequences 24 nucleotides in length having a Nt.BstNBI recognition sequence can be generated based on the following template 5′-NNNNGACTCNNNNNNGAGTCNNNN-3′ (SEQ ID NO: 34). Based on this template, there are 537,824 5′ tail sequences having the following properties: ΔG=−48 Kcal/mole to −62 kcal/mole; ΔΔG<−40 kcal/mole; and GC content 68% to 84%. Of these, 1050 selected sequences are provided, representing 0.2% of the entire sequence space (248,832).

Exemplary 5′ tail region sequences 22 nucleotides in length having a Nt.BstNBI recognition sequence and based on the following template 5′-NNNNGACTCNNNNGAGTCNNNN-3′ (SEQ ID NO: 35). Based on this template, there are 248,832 5′ tail sequences having the following properties: ΔG=−47 Kcal/mole to −55 kcal/mole; ΔΔG<−40 kcal/mole; and GC content 72% to 82%. Of these, 200 selected sequences are provided, representing 0.08% of the entire sequence space (248,832).

### Target Nucleic Acid Molecules

Methods and compositions of the invention are useful for the amplification and/or identification of an EBOV or other viral nucleic acid molecule in a test sample. The target sequences are amplified from virtually any samples that comprises a viral nucleic acid molecule, including a EBOV nucleic acid molecule. In particular, the methods and compositions of the invention are useful for the amplification and/or identification of RNA viruses. In addition to EBOV, exemplary RNA viruses that can be detected using the methods and compositions of the invention include, without limitation, Human Immunodeficiency Virus (HIV), Dengue virus, influenza virus (e.g., influenza B), Bovine Viral Diarrhea virus (e.g., BVDV Genotype 1), Yellow Fever virus, West Nile Virus, Hepatitis C, Lassa virus, Flaviviridae, Arenaviridae, and single-stranded RNA viruses.

Exemplary test samples include body fluids (e.g. bsaliva, sweat, tears, fluids accumulating in a bodily cavity, urine, ejaculate, vaginal secretion, cerebrospinal fluid, lymph, feces, sputum, decomposition fluid, vomit, sweat, breast milk, blood, serum, and plasma), tissue extracts, culture media (e.g., a liquid in which a cell, such as a pathogen cell, has been grown), environmental samples, agricultural products or other foodstuffs, and their extracts, and DNA identification tags. If desired, the sample is purified prior to inclusion in a nicking amplification reaction using any standard method typically used for isolating a nucleic acid molecule from a biological sample.

In one embodiment, primers amplify a target nucleic acid of a pathogen to detect the presence of EBOV or other virus in a sample. For environmental applications, test samples may include water, liquid extracts of building materials (e.g., drywall, ceiling tiles, wall board, fabrics, wall paper, and floor coverings) that may have been exposed to a subject infected with EBOV, environmental swabs, or any other sample.

Methods of the invention provide for the detection of 1×10³, 1×10⁴, 1×10⁵, 1×10⁶, 1×10⁷, or 1×10⁹ copies of EBOV RNA per ml in blood.

### Applications

Target nucleic acid amplification using primers of the invention have characteristics useful for rapid detection of viral (e.g., EBOV) nucleic acid molecules. Compositions and methods of the invention are useful in human diagnostics, where a rapid diagnostic answer is desired (e.g., detectable amplification in under 20, 15, 10, 9, 8, 7, 6, 5 minutes or less). In particular embodiments, the invention provides for the use of an EBOV nicking amplification reaction assay in human or veterinary diagnostics in clinical settings or in the field. In other embodiments, the invention provides for the use of nicking amplification reaction assays in diagnostic field work, where access to thermocycling equipment is unavailable or would be prohibitively expensive. In still other embodiments, the invention provides for the use of nicking amplification reaction assays in a clinical setting where rapid quantitative answers are desired.

### Detectable Oligonucleotide Probes

The present invention provides for the detection of target nucleic acid molecules or amplicons thereof in a nicking amplification reaction using non-amplifiable detectable polynucleotide probes comprising at least one polymerase-arresting molecule (e.g., nucleotide modification or other moiety that renders the oligonucleotide capable of binding a target nucleic acid molecule, but incapable of supporting polymerase extension utilizing the detectable oligonucleotide probe as a target). Without wishing to be bound by theory, the presence of one or more moieties which does not allow polymerase progression likely causes polymerase arrest in non-nucleic acid backbone additions to the oligonucleotide or through stalling of a replicative polymerase (i.e. C3-spacer, damaged DNA bases, other spacer moiety, O-2-Me bases). These constructs thus prevent or reduce illegitimate amplification of the probe during the course of a nicking amplification reaction. This distinguishes them from conventional detection probes, which must be added at the end of the nicking amplification reaction to prevent their amplification.

Conventional detection probes have proven impractical for detecting a nicking amplification reaction in real time. If conventional detection probes are incorporated into the nicking amplification reaction, these conventional detection probes are amplified concurrently with the target. The amplification of these detection molecules masks the detection of legitimate target amplicons due to the number of starting molecules of the detection probe at the start of the reaction.

The invention provides non-amplifiable detectable polynucleotide probe that comprise least one polymerase-arresting molecule. A polymerase-arresting molecule of the invention includes, but is not limited to, a nucleotide modification or other moiety that blocks extension by replicative DNA polymerases, thereby preventing the amplification of detection molecules; but can allow proper hybridization or nucleotide spacing to the target molecule or amplified copies of the target molecule. In one embodiment, a detectable oligonucleotide probe of the invention comprises a 3 carbon spacer (C3-spacer) that prevents or reduces the illegitimate amplification of a detection molecule.

In one embodiment, a detectable oligonucleotide probe comprises one or more modified nucleotide bases having enhanced binding affinity to a complementary nucleotide. Examples of modified bases include, but are not limited to 2′ Fluoro amidites, and 2′OMe RNA amidites (also functioning as a polymerase arresting molecule). Detectable oligonucleotide probes of the invention can be synthesized with different colored fluorophores and may be designed to hybridize with virtually any target sequence. In view of their remarkable specificity, a non-amplifiable detectable polynucleotide probe of the invention is used to detect a single target nucleic acid molecule in a sample, or is used in combination with detectable oligonucleotide probes each of which binds a different target nucleic acid molecule. Accordingly, the non-amplifiable detectable polynucleotide probes of the invention may be used to detect one or more target nucleic acid molecules in the same reaction, allowing these targets to be detected simultaneously. The present invention encompasses the use of such fluorophores in conjunction with the detectable oligonucleotide probes described herein.

Implementation in Hardware and/or Software

The methods described herein can be implemented on general-purpose or specially programmed hardware or software. For example, the methods can be implemented by a computer readable medium. Accordingly, the present invention also provides a software and/or a computer program product configured to perform the algorithms and/or methods according to any embodiment of the present invention. It is well-known to a skilled person in the art how to configure software which can perform the algorithms and/or methods provided in the present invention. The computer-readable medium can be non-transitory and/or tangible. For example, the computer readable medium can be volatile memory (e.g., random access memory and the like) or non-volatile memory (e.g., read-only memory, hard disks, floppy discs, magnetic tape, optical discs, paper table, punch cards, and the like). The computer executable instructions may be written in a suitable computer language or combination of several languages. Basic computational biology methods are described in, for example Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2^{nd} ed., 2001).

The present invention may also make use of various computer program products and software for a variety of purposes, such as probe design, management of data, analysis, and instrument operation. (See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.) Additionally, the present invention may have preferred embodiments that include methods for providing genetic information over networks such as the Internet as shown in U.S. Ser. Nos. 10/197,621, 10/063,559 (US Pub No 20020183936), Ser. Nos. 10/065,856, 10/065,868, 10/328,818, 10/328,872, 10/423,403, and 60/482,389.

### Kits

The invention also provides kits for the amplification of an EBOV or other RNA virus nucleic acid molecule. Such kits are useful for the detection or quantitation of an EBOV or other RNA nucleic acid in a biological sample obtained from a subject. Kits of the present invention may comprise, for example, one or more of reverse transcriptase, DNA polymerases, forward and reverse primers, and one or more nicking enzymes, as described herein, and a detectable probe. Where EBOV or other RNA is to be amplified, one or two nicking enzymes may be included in the kit. Where multiple pathogen sequences are to be amplified, and the templates designed for those target sequences comprise the nicking enzyme sites for the same nicking enzyme, then one or two nicking enzymes may be included. Where the templates are recognized by different nicking enzymes, more nicking enzymes may be included in the kit, such as, for example, 3 or more.

In one aspect, the invention provides a kit for nucleic acid amplification comprising a reverse transcriptase, DNA polymerase; a primary primer, a secondary primer, a nicking enzyme with specificity to a nicking enzyme binding site within the primers, and deoxynucleotide triphosphates (dNTP's) (e.g., in a buffered solution containing components sufficient for amplification. In various embodiments, the primary primer and secondary primer, each have a 3′-end specific recognition region sequence complementary or substantially complementary to the target sequence, where the end specific recognition region comprises one or more 2′ modified nucleotides; a 5′-end tail region containing a nicking enzyme binding site upstream of the 3′-end specific recognition region sequences that is able to dimerize with itself (e.g., self-complementary). In particular embodiments, one or more primers are in a primer-dimer configuration (e.g., produced by heating about Tm and slow cooling).

The kits of the present invention may also comprise one or more of the components in any number of separate containers, packets, tubes (e.g., <0.2 ml, 0.2 ml, 0.6 ml, 1.5 ml, 5.0 ml, >5.0 ml), vials, microtiter plates (e.g., <96-well, 96-well, 384-well, 1536-well, >1536-well), ArrayTape, and the like, or the components may be combined in various combinations in such containers. In various embodiments, the kit further comprises a pair of primers capable of binding to and amplifying a reference sequence. In particular embodiments, the kit comprises one or more primers in a primer-dimer configuration (e.g., produced by heating about Tm and slow cooling). In yet other embodiments, the kit comprises a sterile container which contains the primers; such containers can be boxes, ampules, bottles, vials, tubes, bags, pouches, blister-packs, or other suitable container form known in the art. Such containers can be made of plastic, glass, laminated paper, metal foil, or other materials suitable for holding nucleic acids.

The components of the kit may, for example, be present in one or more containers, for example, all of the components may be in one container, or, for example, the enzymes may be in a separate container from the primers. The components may, for example, be dried (e.g., powder) or in a stable buffer (e.g., chemically stabilized, thermally stabilized). Dry components may, for example, be prepared by lyophilization, vacuum and centrifugal assisted drying and/or ambient drying. In various embodiments, the polymerase and nicking enzymes are in lyophilized form in a single container, and the primers are either lyophilized, freeze dried, or in buffer, in a different container. In some embodiments, the polymerase, nicking enzymes, and the primers are, in lyophilized form, in a single container. In other embodiments, the polymerase and the nicking enzyme may be separated into different containers.

Kits may further comprise, for example, dNTPs used in the reaction, or modified nucleotides, cuvettes or other containers used for the reaction, or a vial of water or buffer for re-hydrating lyophilized components. The buffer used may, for example, be appropriate for both polymerase and nicking enzyme activity.

The kits of the present invention may also comprise instructions for performing one or more methods described herein and/or a description of one or more compositions or reagents described herein. Instructions and/or descriptions may be in printed form and may be included in a kit insert. A kit also may include a written description of an Internet location that provides such instructions or descriptions.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1. One-Step and Two-Step Real-Time Reverse Transcription-Isothermal Amplification Assays for EBOV

A one-step reaction refers to a reverse transcriptase (RT) reaction in which reverse transcription and amplification occur in a single reaction protocol. A two-step reaction refers to a reverse transcriptase reaction in which the reverse transcription is carried out first; followed by a transfer to a second amplification reaction.

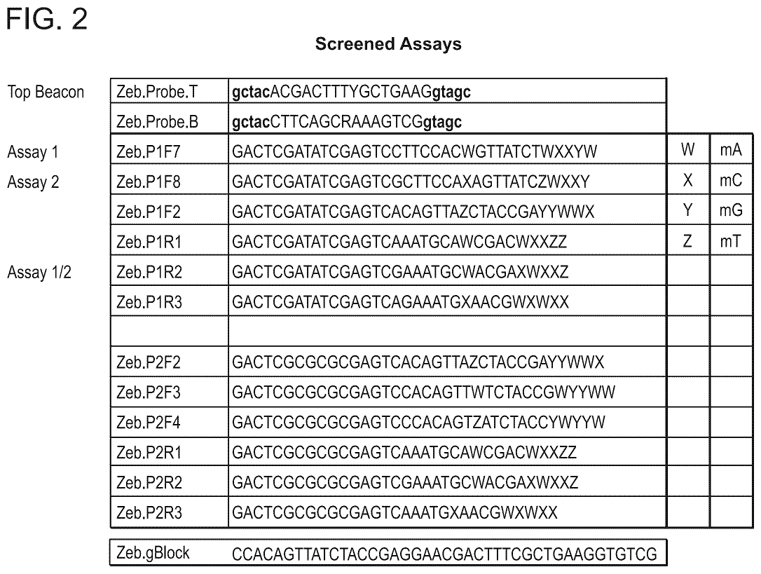

Assays 1 and 2 were tested for their ability to detect an EBOV polynucleotide EBOV (FIG. 1). Primers for these assays are shown at FIG. 2.

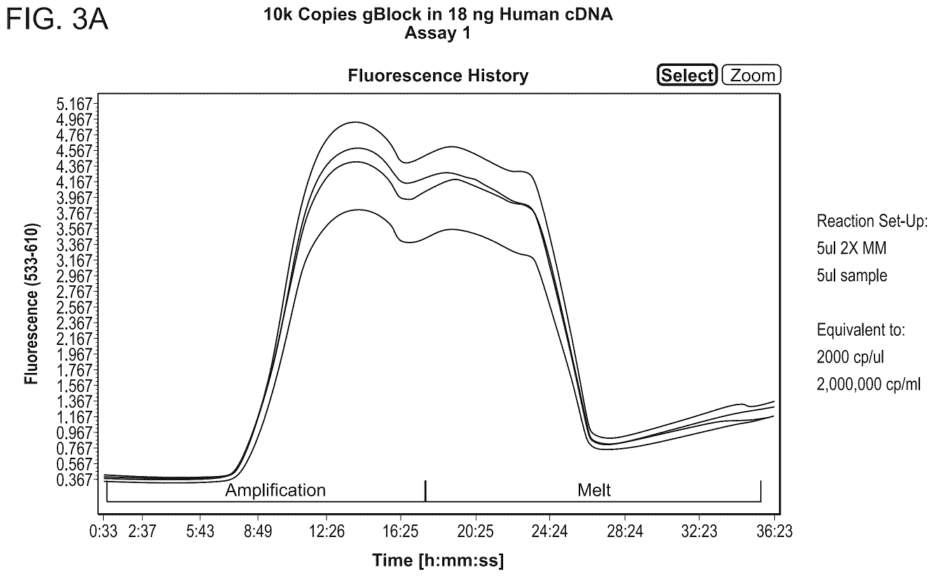

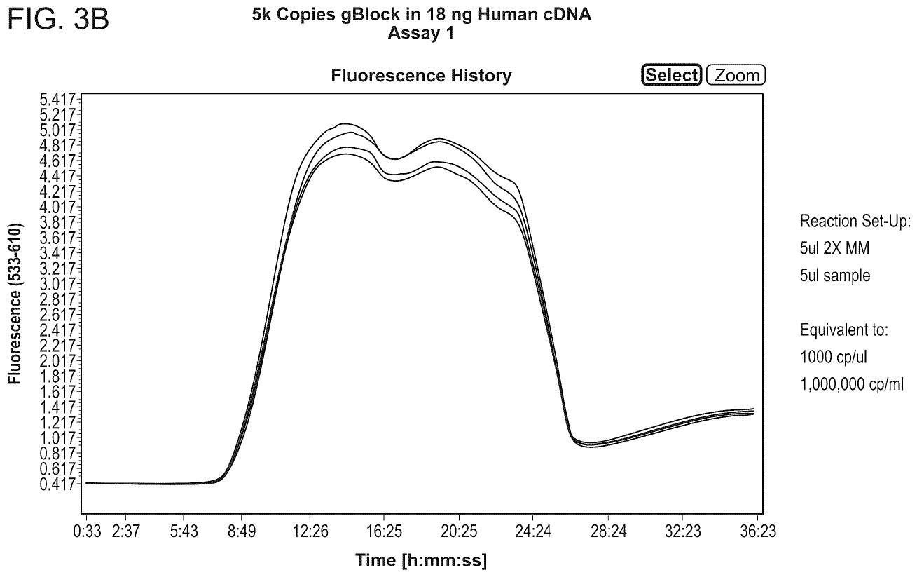

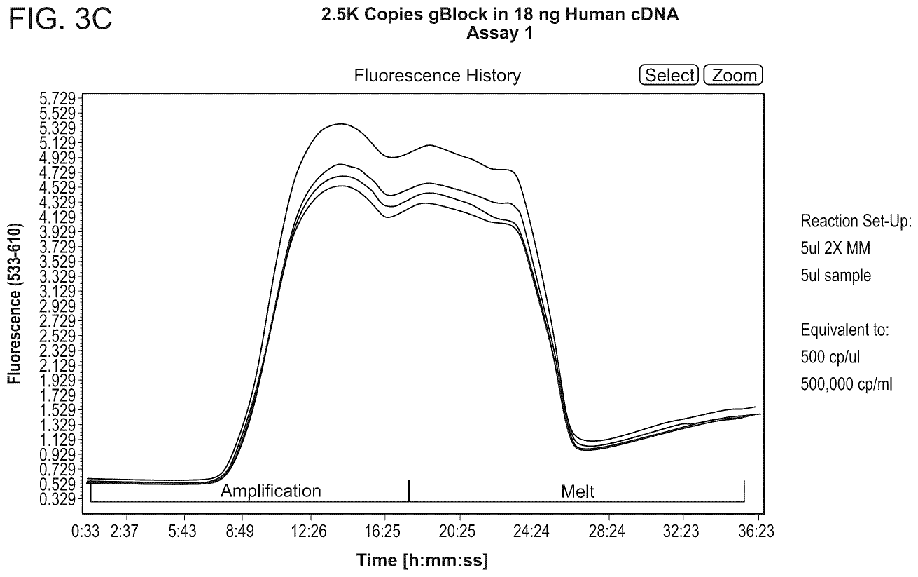

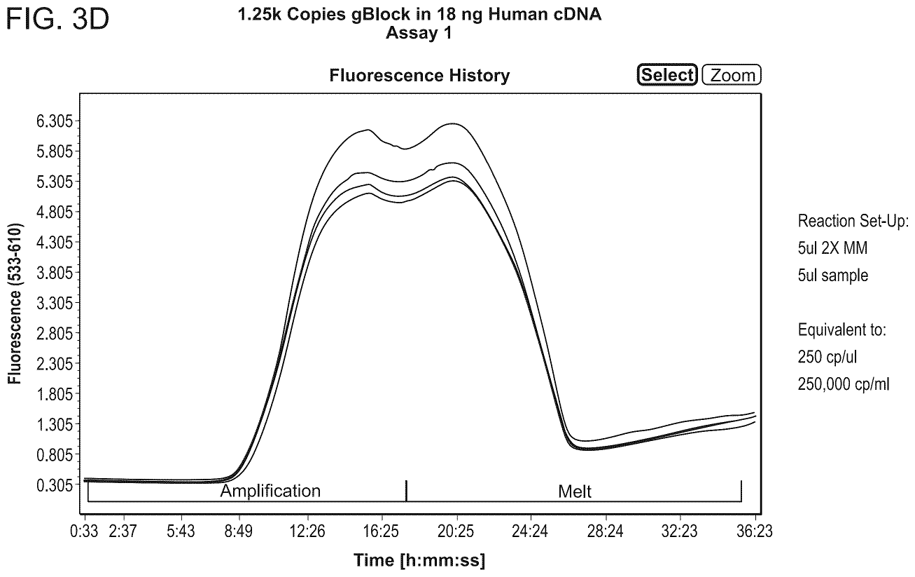

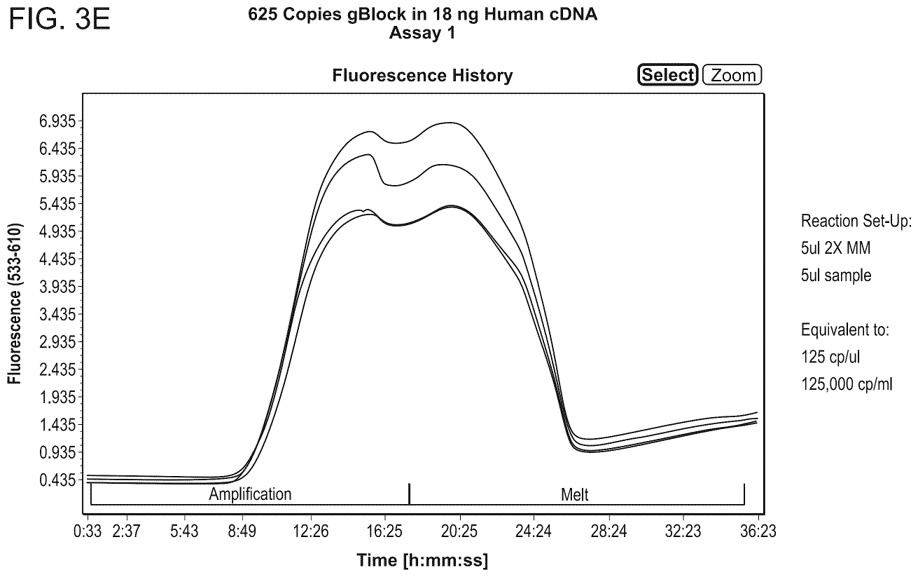

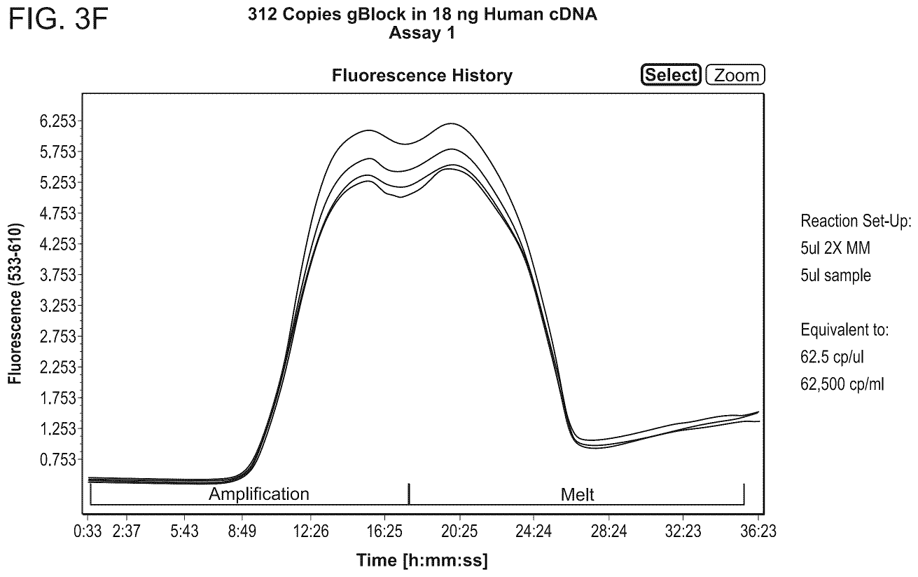

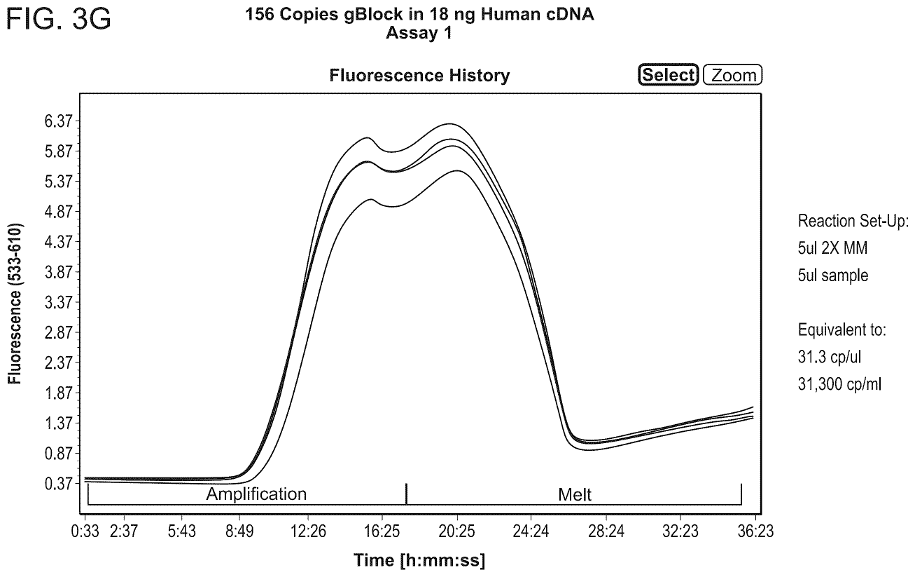

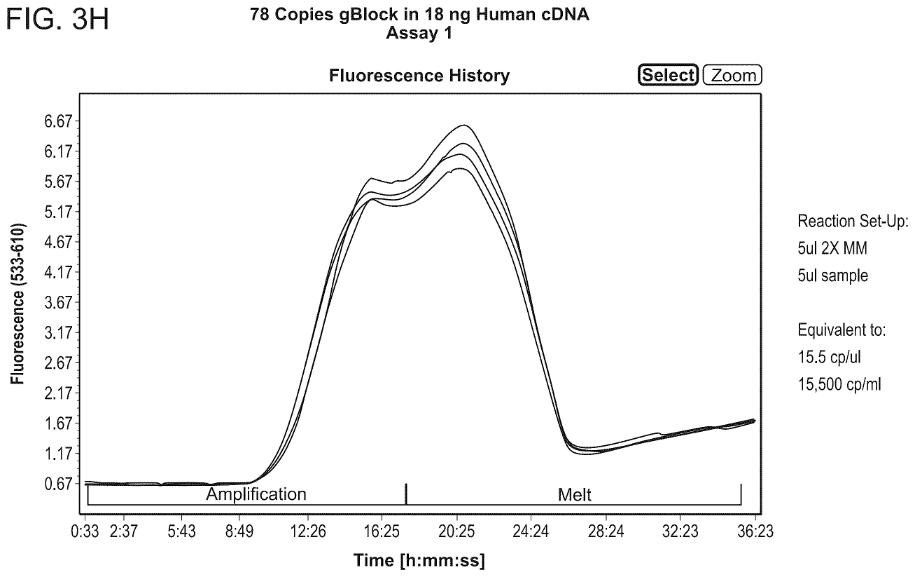

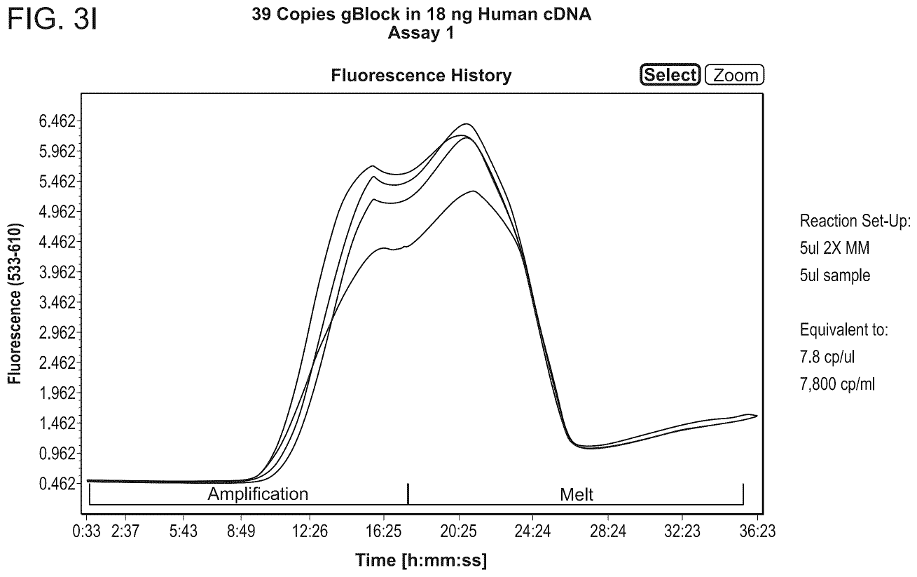

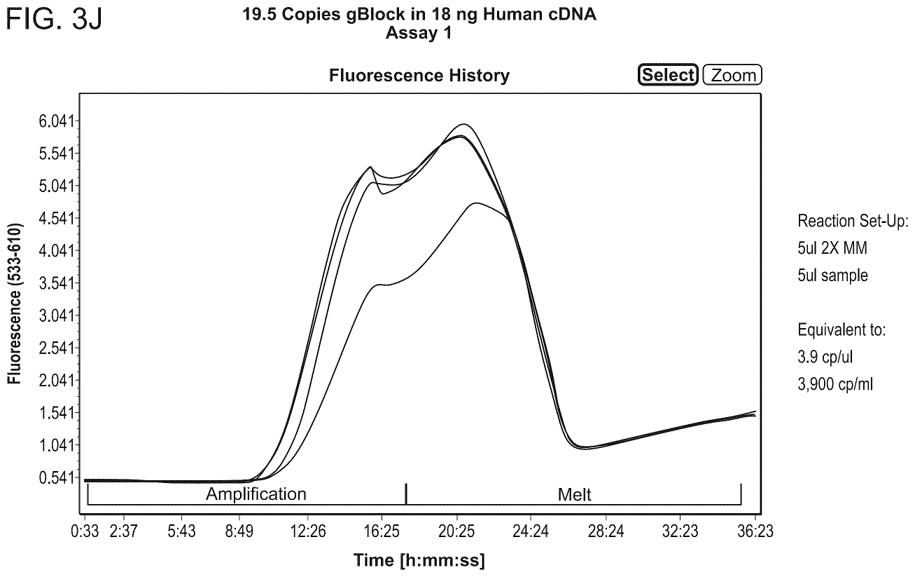

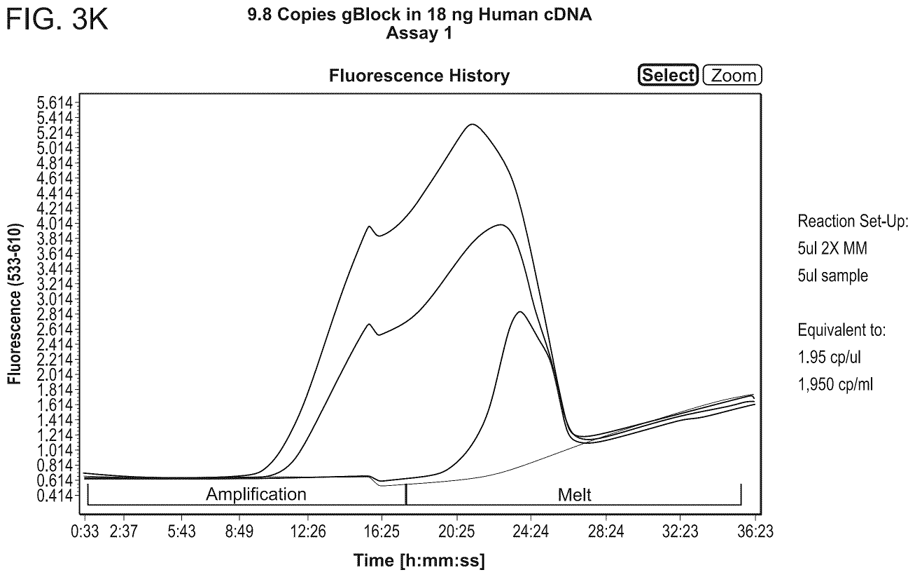

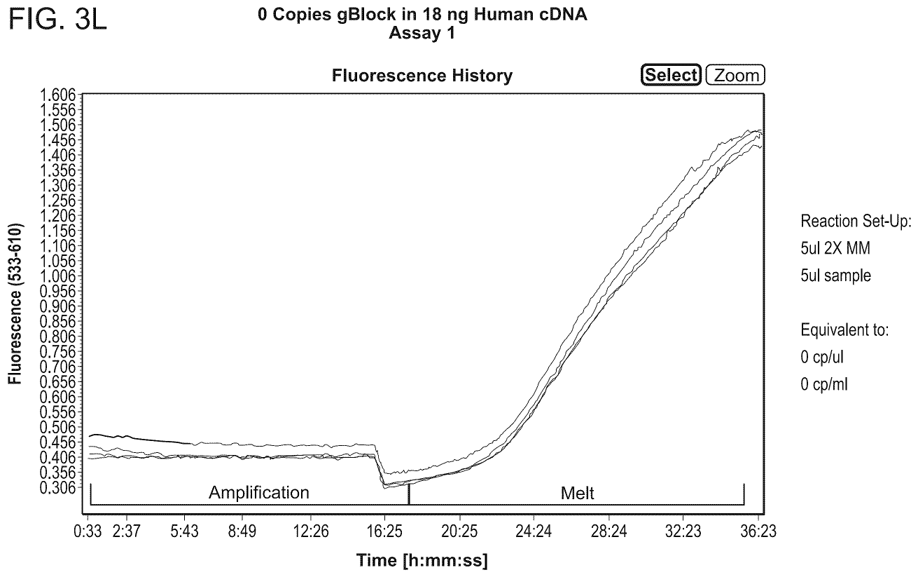

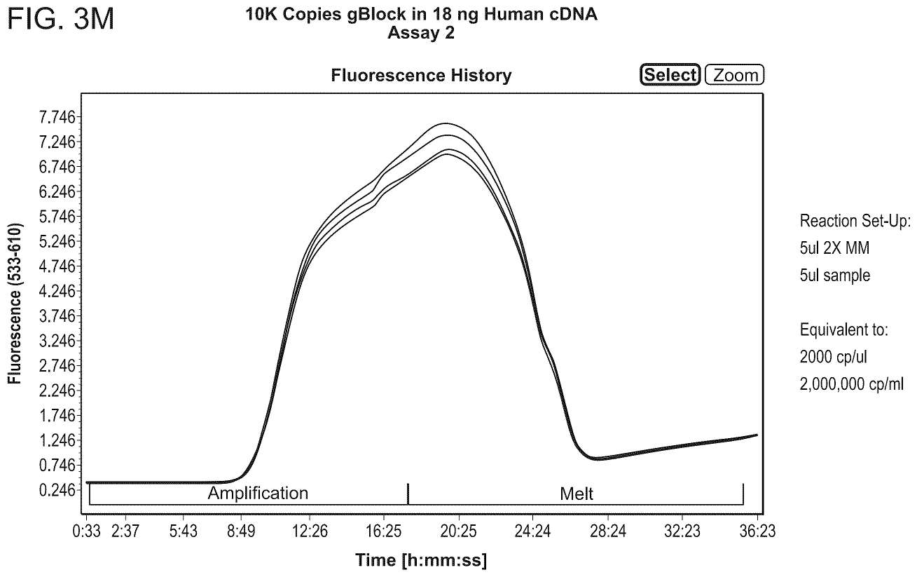

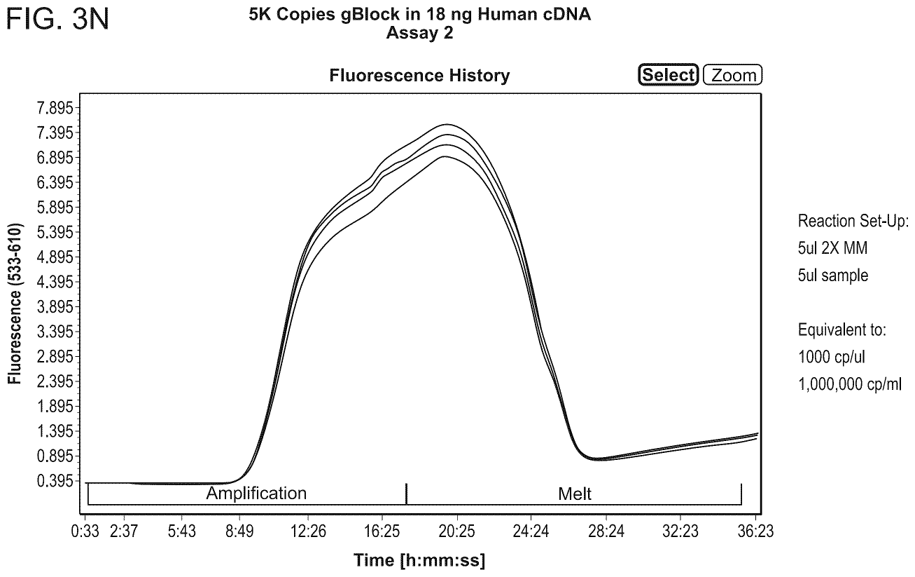

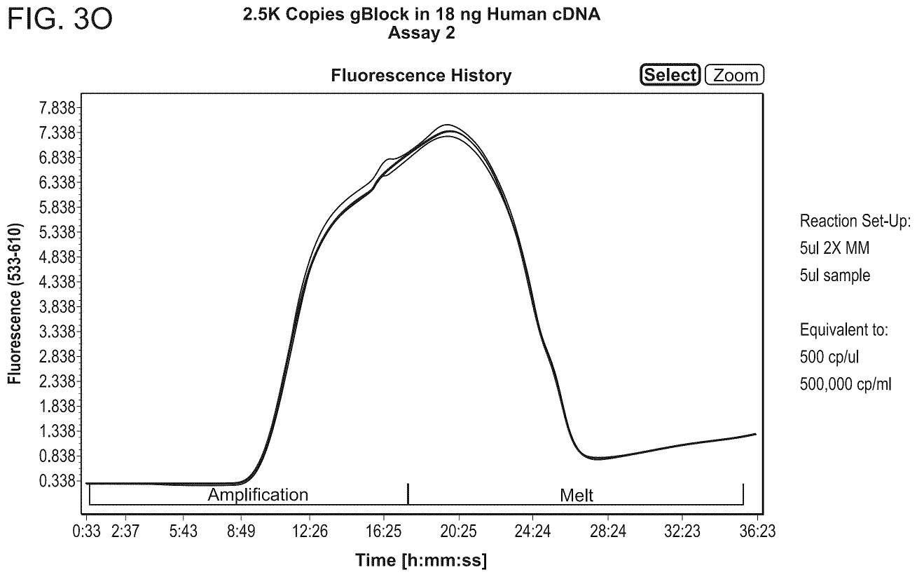

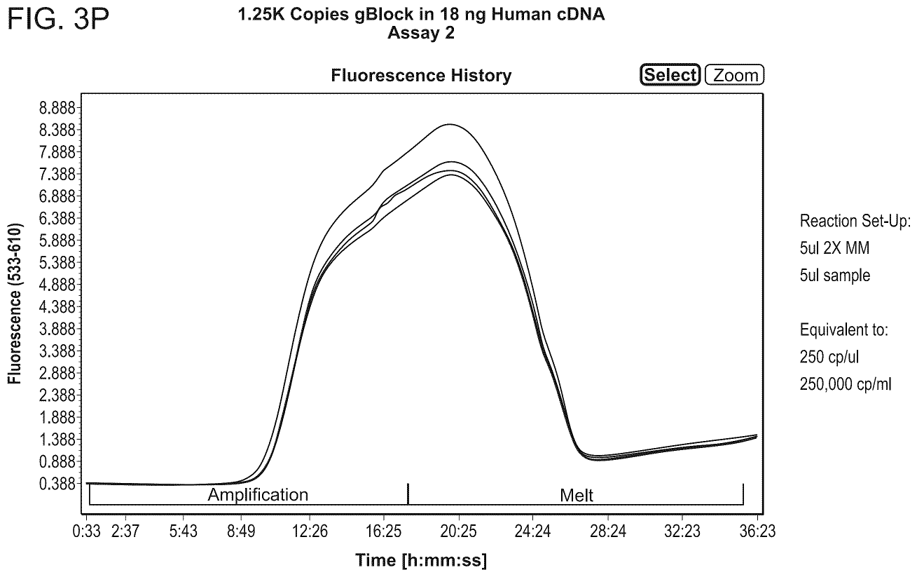

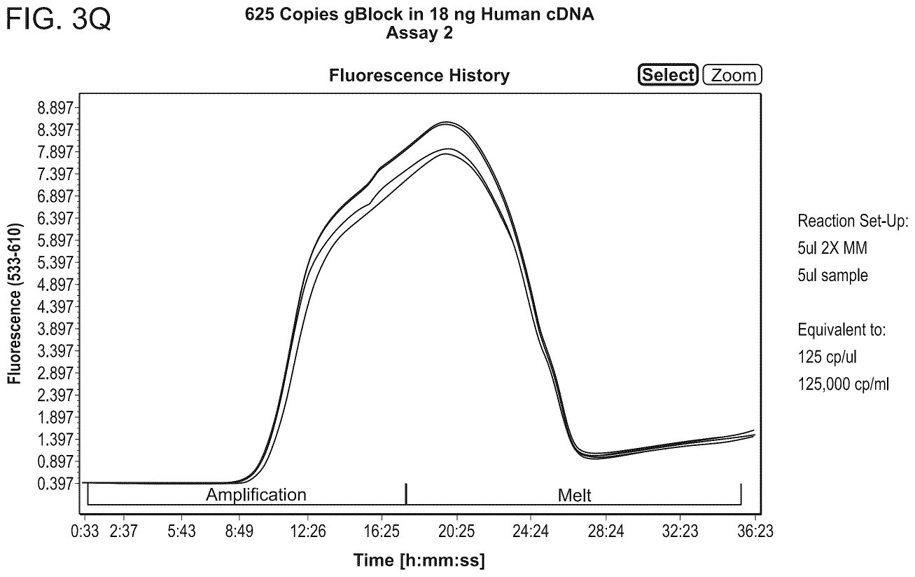

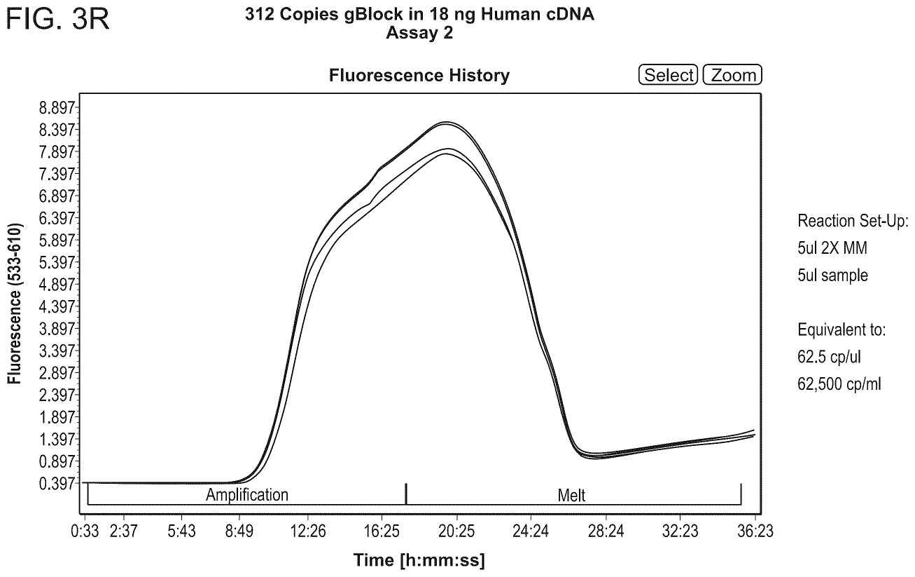

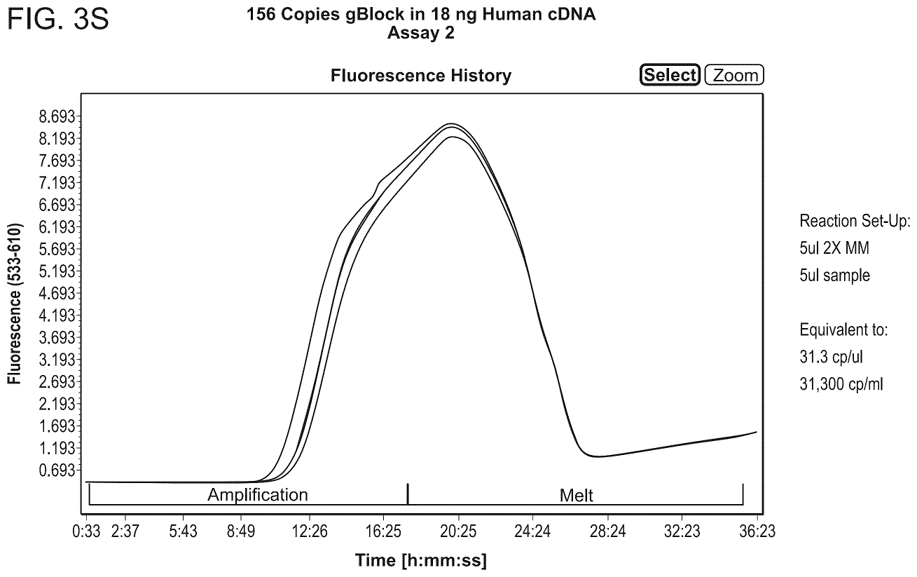

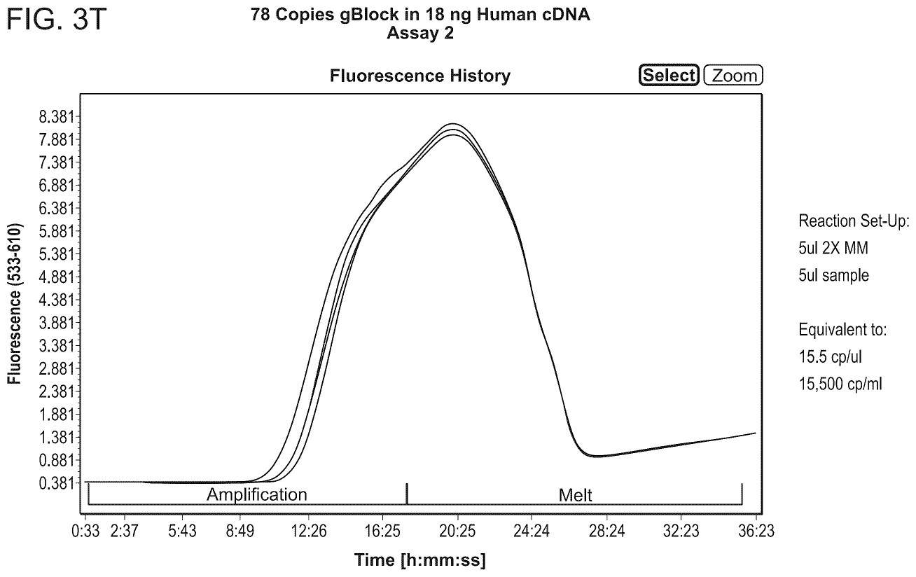

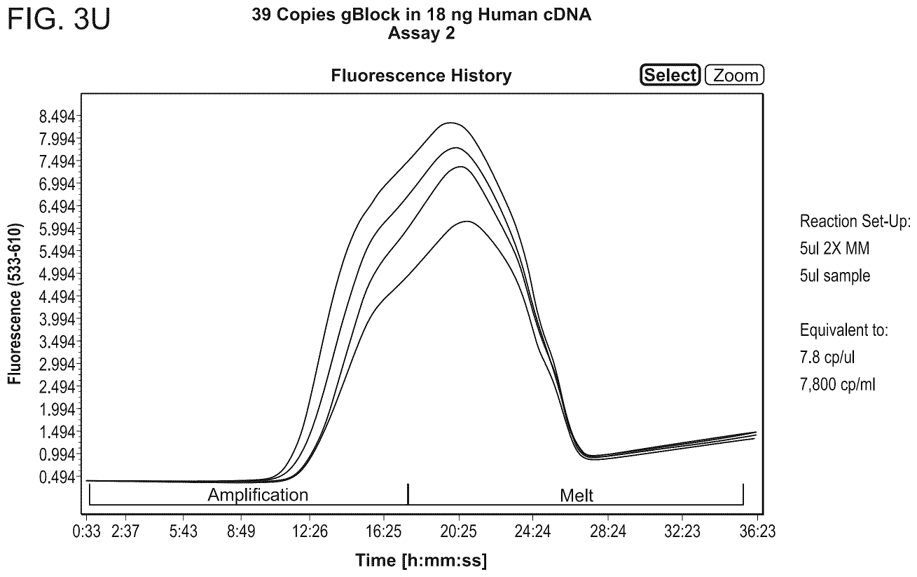

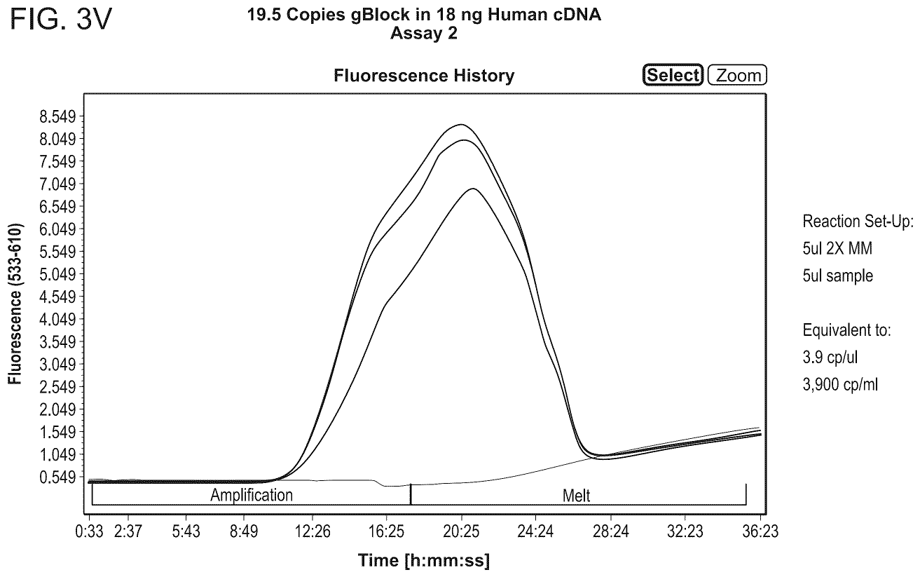

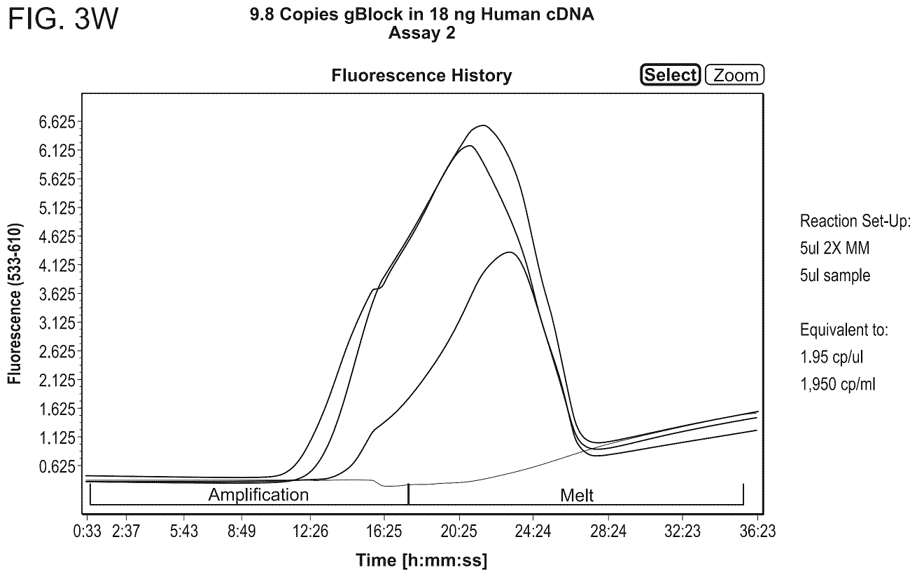

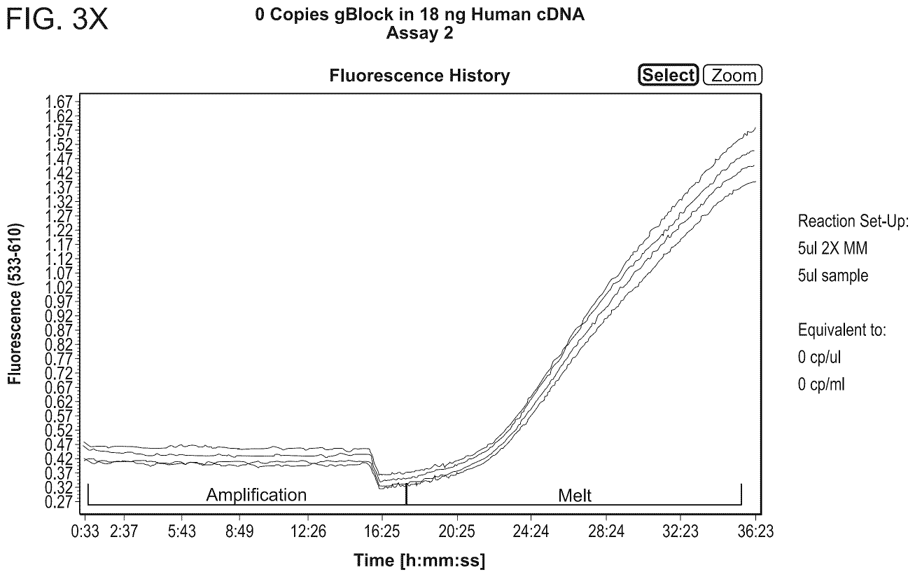

FIGS. 3A-3X show amplification results obtained with Assay 1 and Assay 2. The assays were used to detect copies of EBOV gBlock in a background of human cDNA.

Reaction conditions are specified.

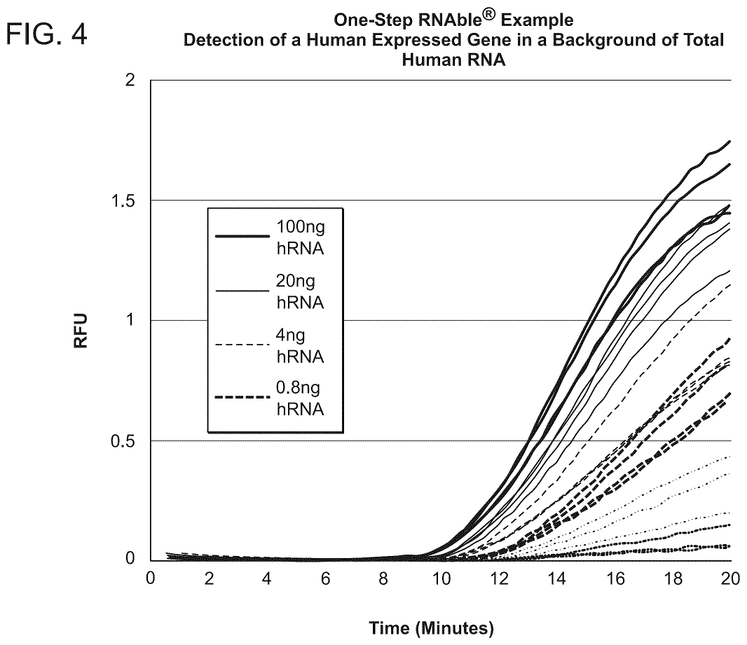

FIG. 4 shows detection of EBOV in a background of total human RNA in a one-step assay.

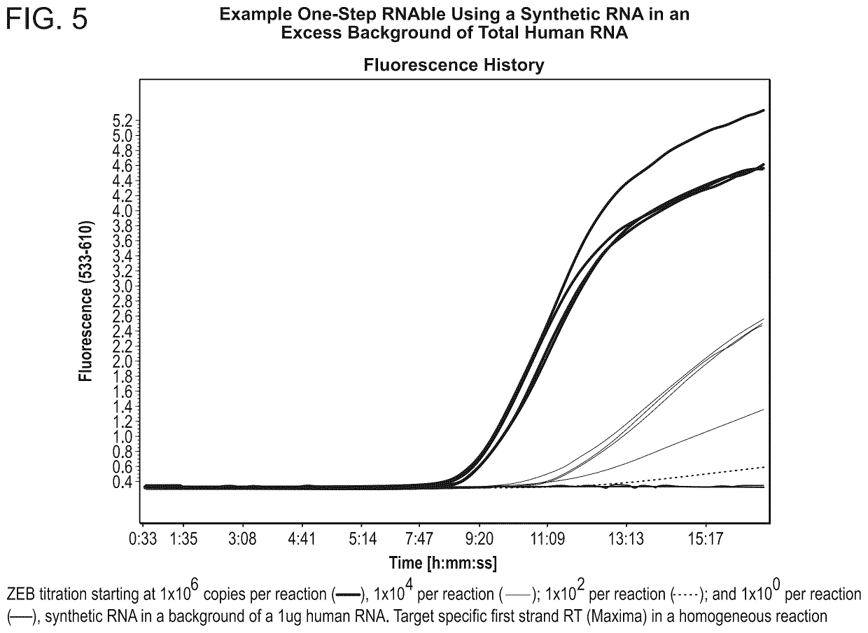

FIG. 5 shows detection of synthetic EBOV RNA in a background of total human RNA in a one-step assay.

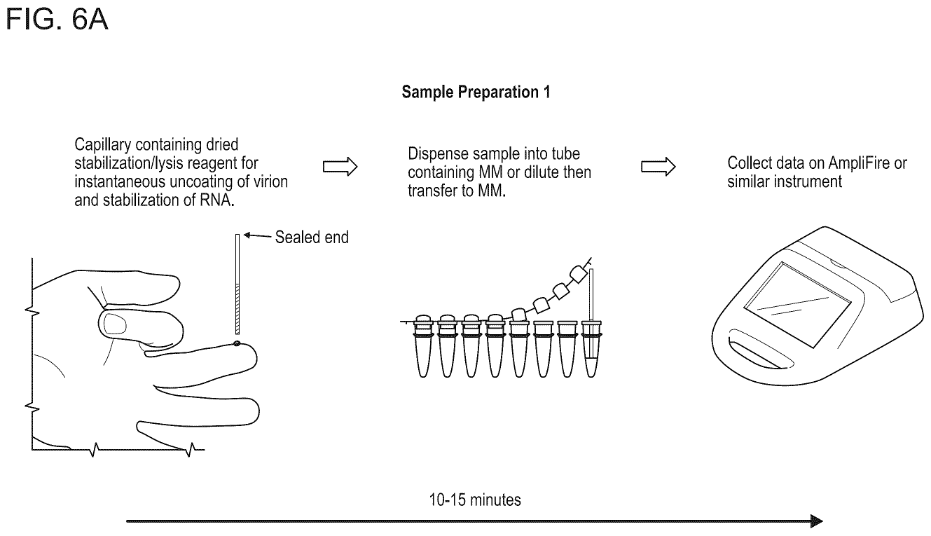

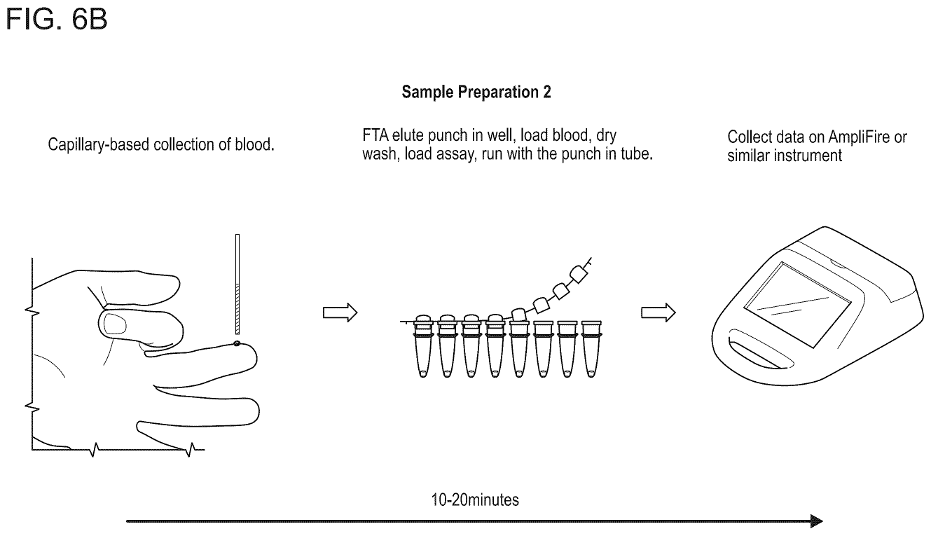

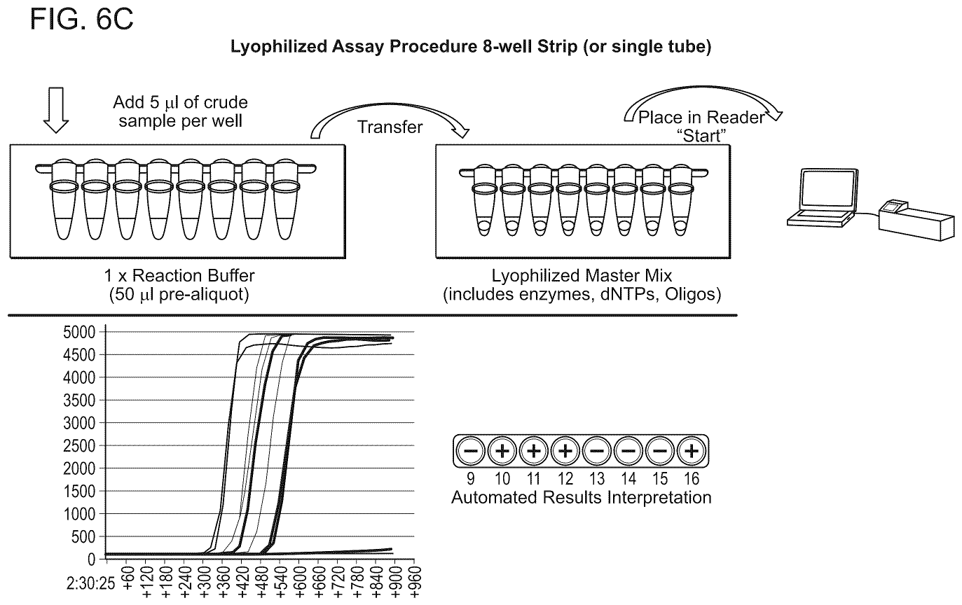

FIGS. 6A-6C provide flow charts illustrating sample processing for use in an amplification and detection instrument.

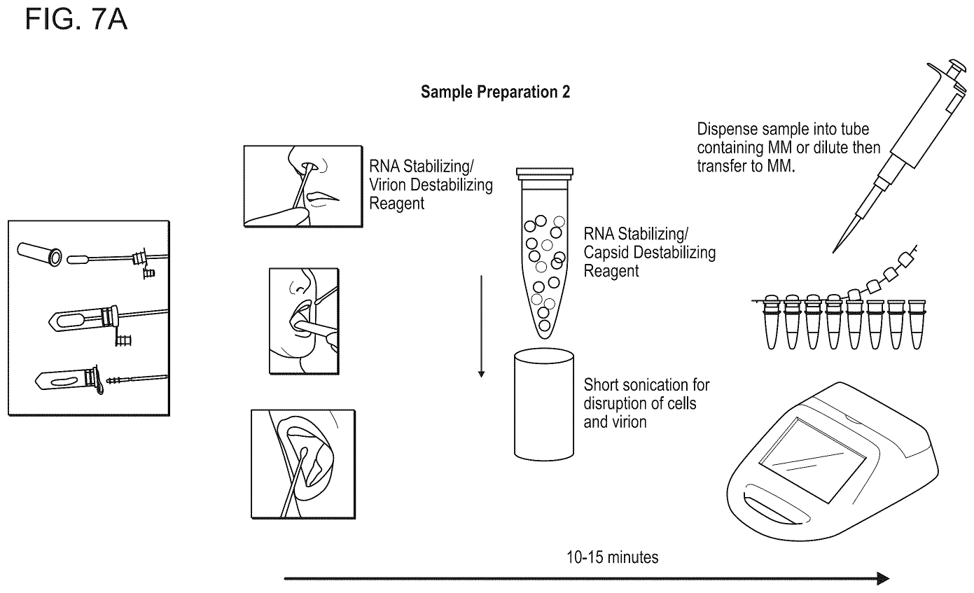

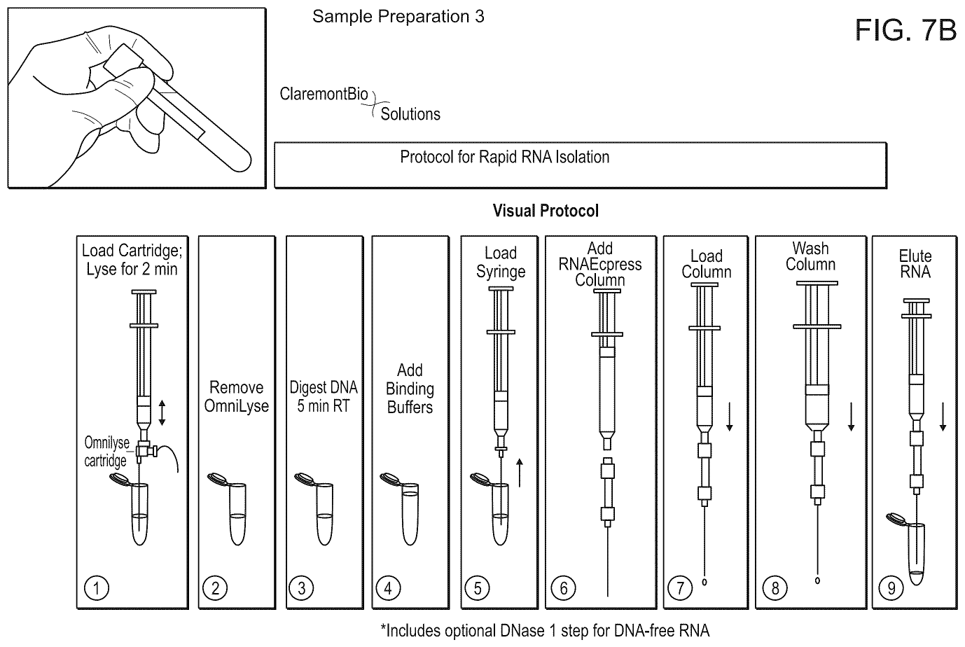

FIGS. 7A and 7B show sample preparation methods from swabs and blood, respectively.

In one working example, Ebola virus was detected using the following primers and probe sequences:

| | | |
|---|---|---|
| Forward | GACTCGATATCGAGTCGCTTCCA[MeOC]AGTTATC | SEQ ID NO: 1 |
| Primer | [MeOU][MeOA][MeOC][MeOC][MeOG] | |
| Reverse | GACTCGATATCGAGTCGAAATGC[MeOA]ACGA[MeOC] | SEQ ID NO: 2 |
| Primer | [MeOA][MeOC][MeOC][MeOU] | |
| Probe | gctacACGACTTTYGCTGAAGgtagc | SEQ ID NO: 3 |
| External | CTTCTTAGCTTGGGGCAGTATCA | SEQ ID NO: 36 |
| Primer | | |

Primers: [MeON] indicates methoxy base
Probe sequence: lowercase = stems, uppercase = recognition
1:5 1000 nM 0.3 U/μl nicking enzyme

In the sequences above, GAGTC is the nicking enzyme recognition site.

Pyrimidine provides for degeneracy detection of Ebola strains including Zaire.

Synthetic DNA target (SEQ ID NO: 37):
AAGATGACTGCAGGAGTCAATGCGCAGTTGGTCCCGGCAGACCAGGCGAAC
ATTACCGAATTTTACAACAAGTCCCTTTCATCCTACAAGGAGAATGAGGAG
AACATCCAGTGTGGGGAGAACTTCATGGACATGGAGTGCTTCATGATTCTG
AACCCCAGTCAGCAGCTGGCAATTGCCGTCTTGTCTCTCACACTGGGCACC
TTCACAGTTCTGGAGAACTTGCTGGTGCTGTGTGTCACCACAGTTATCTAC
CGAGGAACGACTTTCGCTGAAGGTGTCGTTGCATTTCTGATTCCTTCACTC
CCGCAGCCTCCGCTGCCGGCCCTCTTACCACTTCATCATTAGCCTGGCCGT
GGCCGACCTTCTGGGGAGTGTCATTTTTGTCTACAGCTTTGTTGACTTTCA
TGTGTTCCACCGCAAGGACAGCCCCAACGTCTTTCTCTTCAAATTGGGTGG
GGTCACCGCCTCCTTCACGGCCTCTGTAGGCAGCCTCTTCC
Synthetic RNA target (SEQ ID NO: 38):
rGrArCrUrGrCrArGrGrArGrUrCrUrGrCrUrGrCrUrUrCrCrArCr
ArGrUrUrArUrCrUrArCrCrGrArGrGrArArCrGrArCrUrUrUrCrG
rCrUrGrArArGrGrUrGrUrCrGrUrUrGrCrArUrUrUrCrUrGrArUr
UrCrCrUrUrCrArCrUrCrCrCrG

Both 1-step and 2-step reactions contained a final concentration of 166.7 nM forward primer and a final concentration of 833.3 nM reverse primer with 200 nM concentration of Probe and 0.1×SYBR green (final concentration). In addition both 1 and 2 step reactions contained a final concentration of 1× Extract Buffer 2, comprising Tris pH 8.0, NH₄⁺, Na⁺, and Mg²⁺; dNTPs; 0.4 U/μl BST polymerase; and 0.3 U/μl Nt.BSTnbi nicking enzyme. In addition to these components; the 1 step reactions contained 10 U/μl of Maxima Reverse Transcriptase enzyme; 1.0 U/μl of an RNAse inhibitor (SUBERase IN by life technologies). Synthetic RNA had 1.0 U/μl of RNAse inhibitor added as well to prevent degradation. All water used was purchased nuclease free.

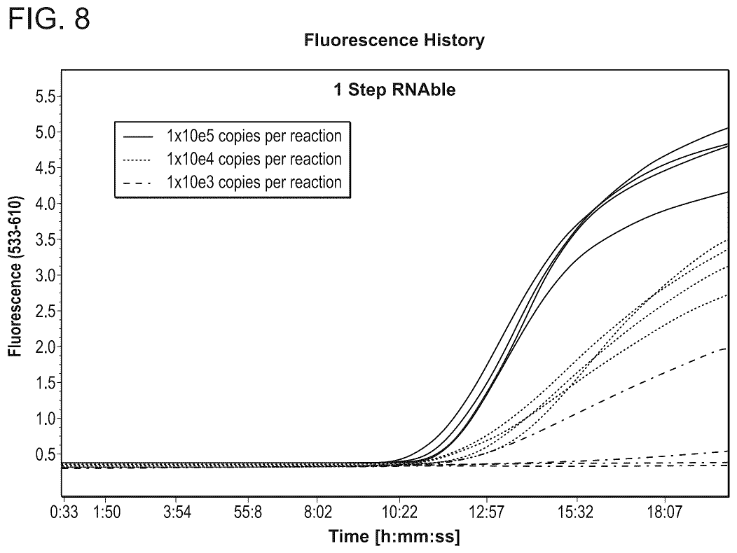

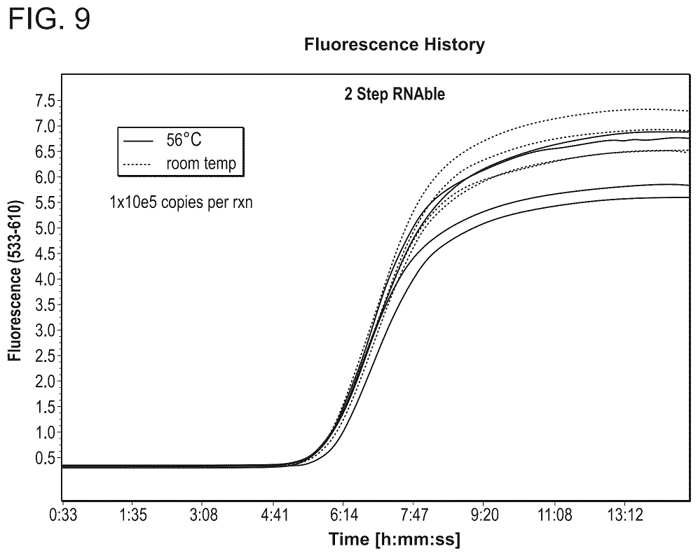

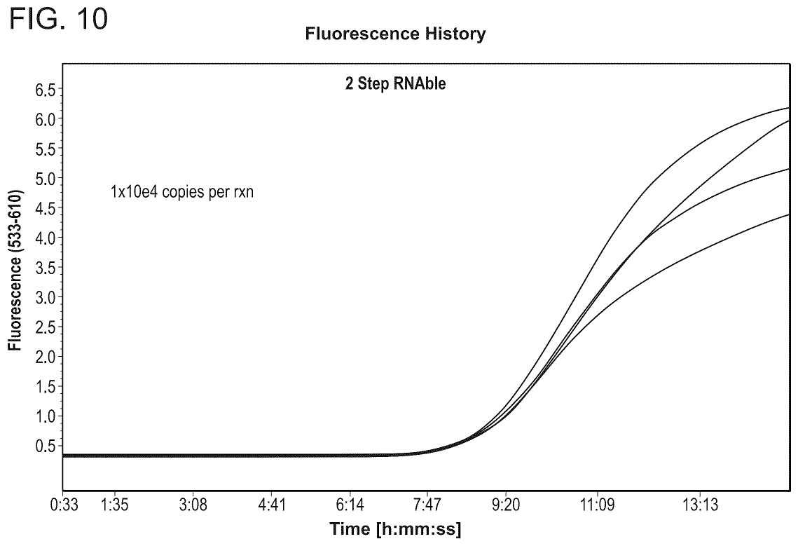

Reactions were mixed on ice and kept cold until run on the Roche LC480. The Roche LC480 was run under a two color detection to detect the calfluor red 610 beacon signal (Abs 590 nm/Em 610 nm) and the SYBR green signal (Abs 495 nm/Em 520 nm).
1-Step reactions were carried out at 56° C. for 20 minutes. Results are shown at FIG. 8.
2-step reactions were carried out with the reverse transcriptase step at 56° C. for 5 minutes (in a heat block) or at room temperature for 5 minutes following the setup condition outlined by New England Biolabs (www.neb.com) (FIG. 9). Both these RT temperatures produced signal for 1×10⁵ copies of target per reaction. The reverse transcriptase step was followed by an amplification step at 56° C. for 15 minutes on the LC480. Results of this assay are shown at FIG. 10. The copy number indicated per reaction was determined using the suppliers' calculations.

### Example 2. Detection of a Target RNA in a Complex RNA Mixture

To determine whether the 1-step and 2-step reactions could detect a target RNA in complex mixtures of RNA molecules, assays were designed for the detection of RPPH1 (RNase P RNA Component) in total human RNA. In one working example, RPPH1 was detected using the following primers and probe sequences:

| | | | |
|---|---|---|---|
| Forward | RPPH1.Fc | GACTCGATATCGAGTCCACGAGCmUG | SEQ ID NO: 39 |
| Primer | | AGTGCmGmUmCmCmUmG | |
| Reverse | RPPH1.Rc | GACTCGATATCGAGTCAGACCTTmCC | SEQ ID NO: 40 |
| Primer | | CAAGGmGmAmCmAmU | |
| Probe | RPPH1.Probe.T | CCACGCCTGTCACTCCACTCCGCGTGG | SEQ ID NO: 41 |
| External | rpph1extprimR | CCTCTGGCCCTAGTCTCAG | SEQ ID NO: 42 |
| Primer | | | |

Primers: mN indicates methoxy base

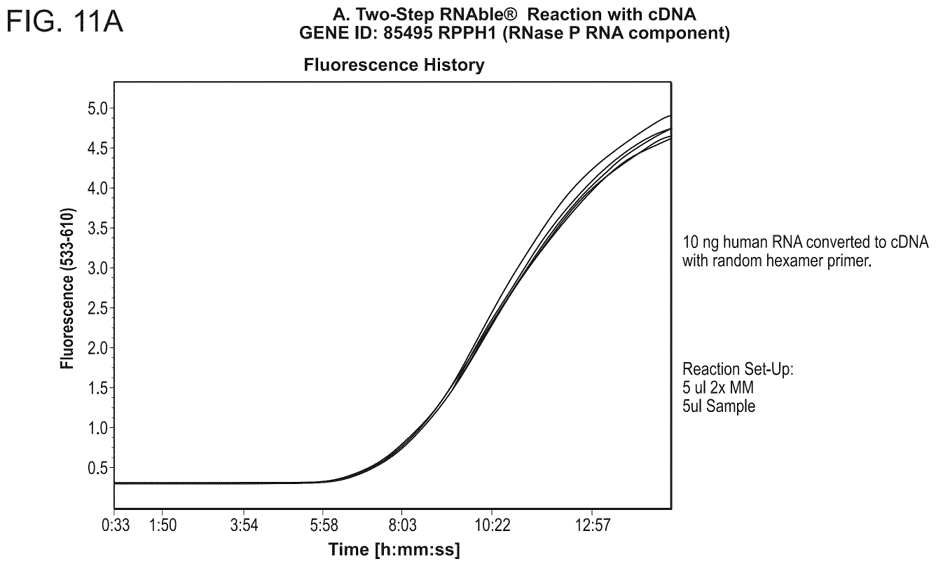

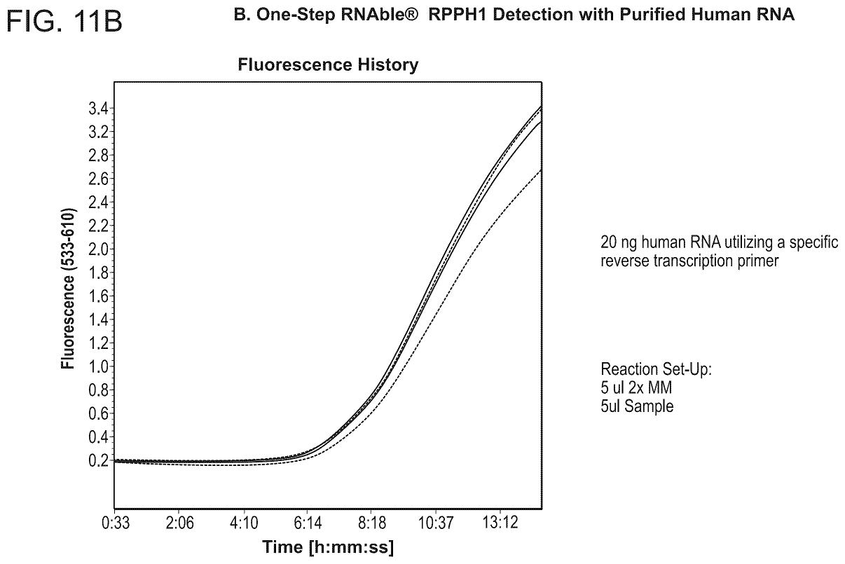

In a 2-step reaction, human RNA (10 ng) was converted to cDNA with random hexamer primer and RPPH1 was detected by amplification of a target specific sequence (FIG. 11A). In a 1-step reaction, human RNA (20 ng) was detected by amplification of a target specific sequence using specific reverse transcription primers (FIG. 11B).

### Example 3: Detection of Ebola Virus in a Biological Sample

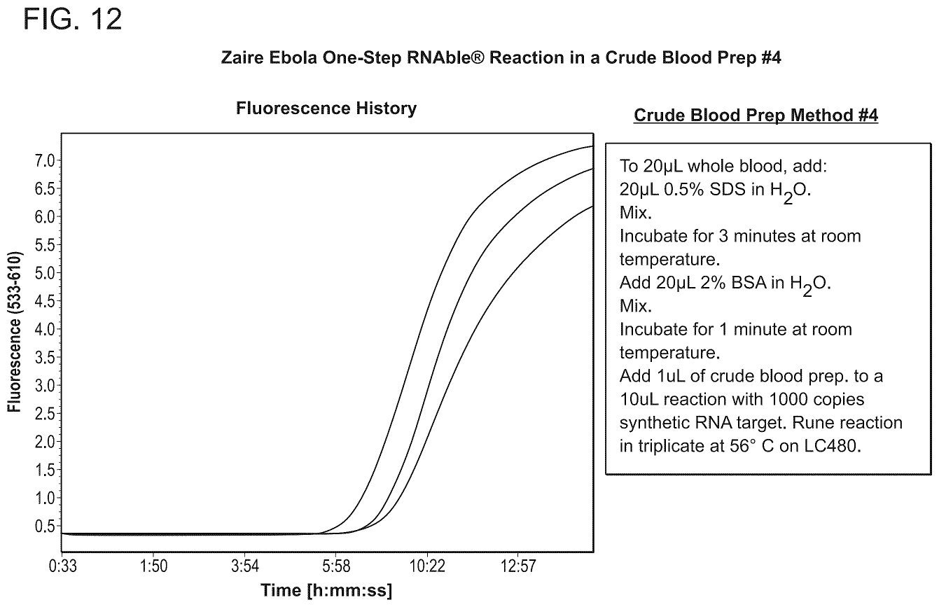

In another working example, a synthetic Ebola virus RNA target was detected when mixed with a crude blood preparation using the 1-step assay. The crude blood preparation was prepared by mixing whole blood (20 μl) and sodium dodecyl sulfate (0.5% SDS; 20 μl) and incubating the mixture at room temperature (3 min.). After incubation, bovine serum albumin was added (2% BSA; 20 μl) and the resulting mixture was incubated at room temperature (1 min.). The crude blood preparation (1 μl) was spiked with a synthetic Ebola virus RNA target (1000 copies). Reactions were run in triplicate at 56° C. on a Roche LC480. Results are shown at FIG. 12.

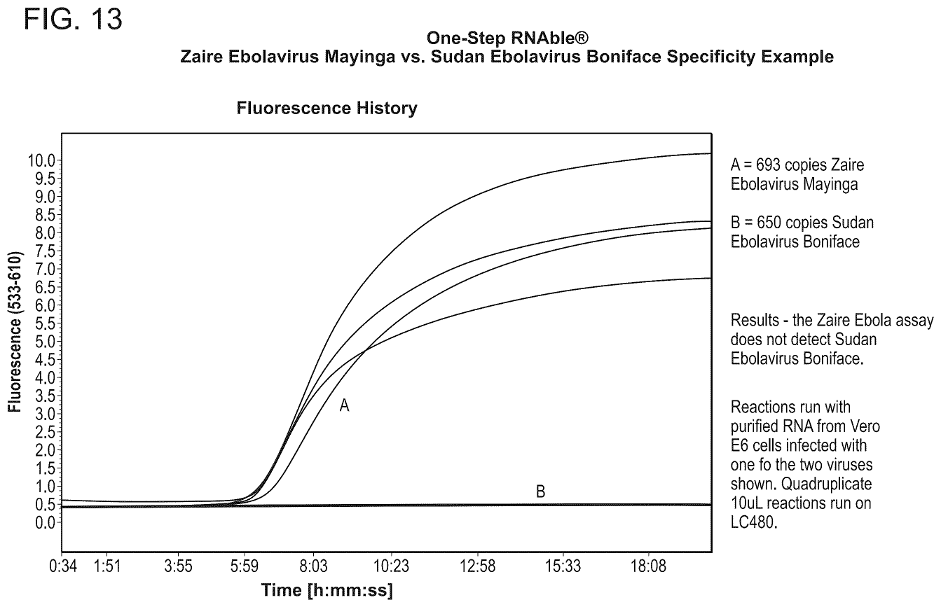

In an additional experiment, the 1-step assay was able to distinguish between Zaire Ebolavirus Mayinga and Sudan Ebolavirus Boniface RNA molecules. Vero E6 cells were infected with Zaire Ebolavirus Mayinga or Sudan Ebolavirus Boniface virus and viral RNAs were purified. Sets of reactions were run using purified RNA Zaire Ebolavirus Mayinga (683 copies) or Sudan Ebolavirus Boniface virus (650 copies). For each set, quadruplicate reactions (10 μl) were run on a Roche LC480. The assay detected Zaire Ebolavirus Mayinga RNA (FIG. 13; set of curves denoted A) and did not detect Sudan Ebolavirus Boniface RNA (FIG. 13; set of curves denoted B). Thus, the Zaire Ebola assay was specific for the detection of Zaire Ebola Mayinga.

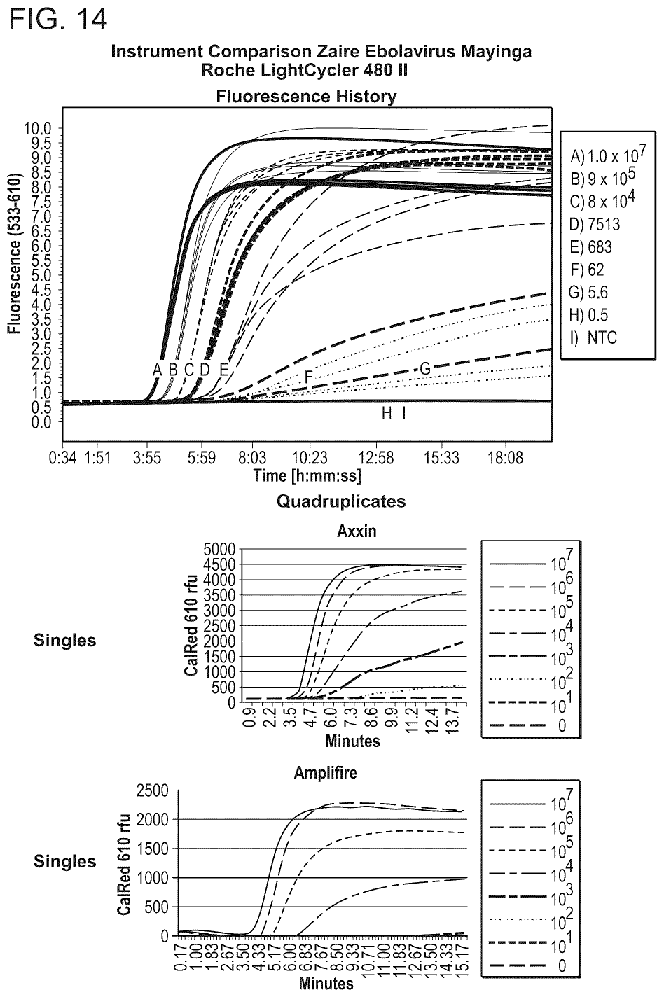

An instrument comparison was run using various dilutions of Zaire Ebolavirus Mayinga RNA from about 10¹-10⁷ copies of target RNA, including a no target control (NTC) sample. Instruments tested included the Roche Lightcycler 480 II, Axxin Detector, and Douglas Scientific Amplifire amplification and detection instruments. While some instrument variability was observed, all instruments could detect copies of EBOV RNA over a wide range (FIG. 14).

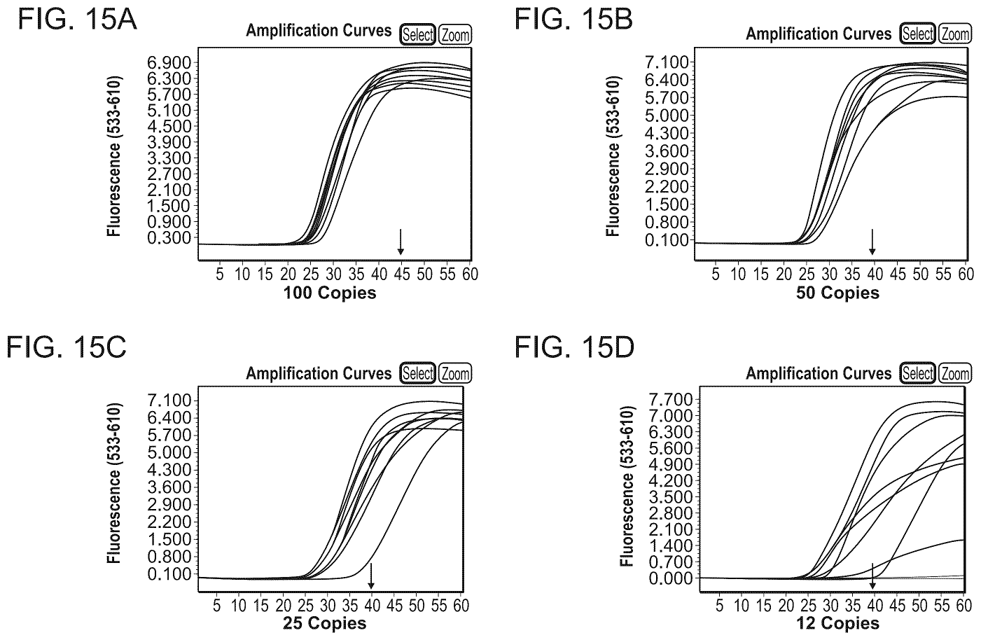

To determine a lower limit of detection for the 1-step EBOV assay, serially diluted samples containing 100, 50, 25, and 12 copies of Zaire Ebolavirus Mayinga RNA were tested. The 1-step reactions (10 μl) were run on the Roche LightCycler 480 using at least 10 technical replicates for each dilution. Twenty (20) technical replicates were run for the 100 and 50 copy reactions. Forty (40) technical replicates were run for the 25 copy reactions. A 100% (20/20) detection rate was observed for the detection of 100 and 50 copies per reaction (FIGS. 15A and 15B). A 95% (38/40) detection rate was observed for the detection of 25 copies per reaction (FIG. 15C). Thus, these results show the sensitivity and specificity of the EBOV assay, even when performed as a 1-step assay.

### Example 4: Detection of Human Immunodeficiency Virus (HIV) in a Biological Sample

In one working example, human immunodeficiency virus (HIV) was detected in a 1-step RNAble® assay targeting a gag protein sequence. Purified HIV RNA (subtype C) was obtained (SeraCare), and known quantities of the HIV RNA, as quantified by COBAS TaqMan HIV-1 v2.0 test, were tested in the 1-step HIV RNAble® assay. The following primers and probe sequences were used:

Hgag (HIV gag target)

| Primer | Sequence | SEQ ID NO |
|---|---|---|
| Hgag.F2a | GACTCGATATCGAGTCTGACTAGmCGGAGGmCmTm | SEQ ID NO: 6 |
| | AmGmAmAmG | |
| Hgag.F2b | GACTCGATATCGAGTCTGACTAGmCAGAGGmCmTm | SEQ ID NO: 7 |
| | AmGmAmAmG | |
| Hgag.R1a | GACTCGATATCGAGTCTATTGACmGCTCmTmCmGm | SEQ ID NO: 8 |
| | CmAmC | |
| Hgag.R1b | GACTCGATATCGAGTCTACTGACmGCTCmTmCmGm | SEQ ID NO: 9 |
| | CmAmC | |
| Hgag.rt3.subC* | GCATCTAATTTTTCGCC (external primer) | SEQ ID NO: 43 |
| Hgag.probe.T | cgcaagGGAGAGAGATGGGTGcttgcg | SEQ ID NO: 10 |

Primers: mN indicates methoxy base
Probe sequence: lowercase = stems, uppercase = recognition
*External primer sequence is specific to HIV subtype C (for the purified RNA sample used)

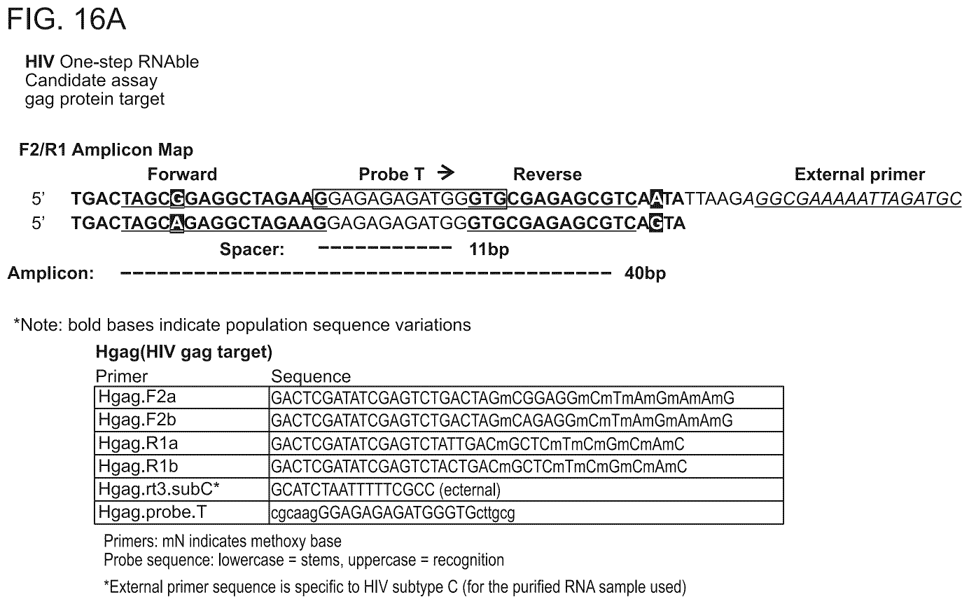

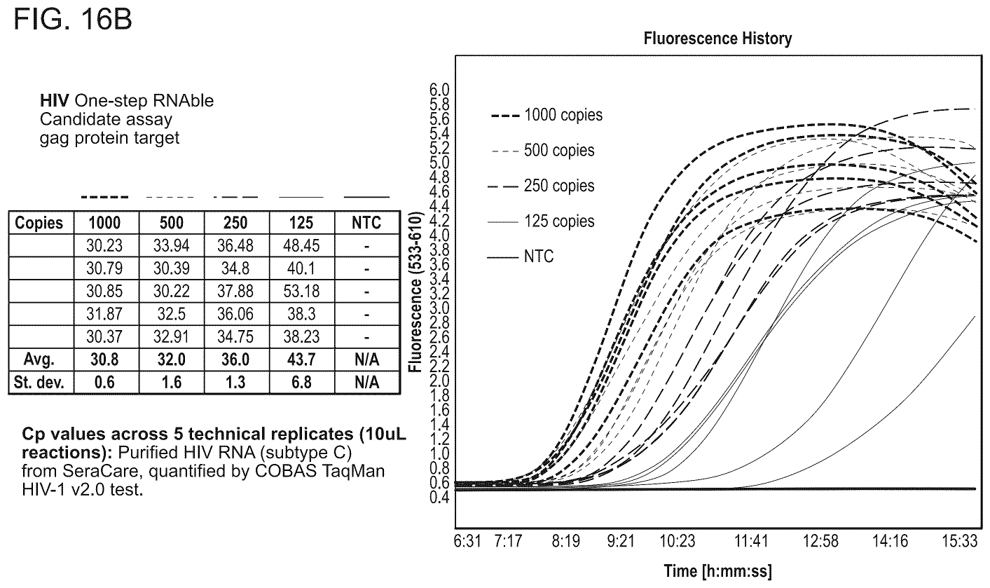

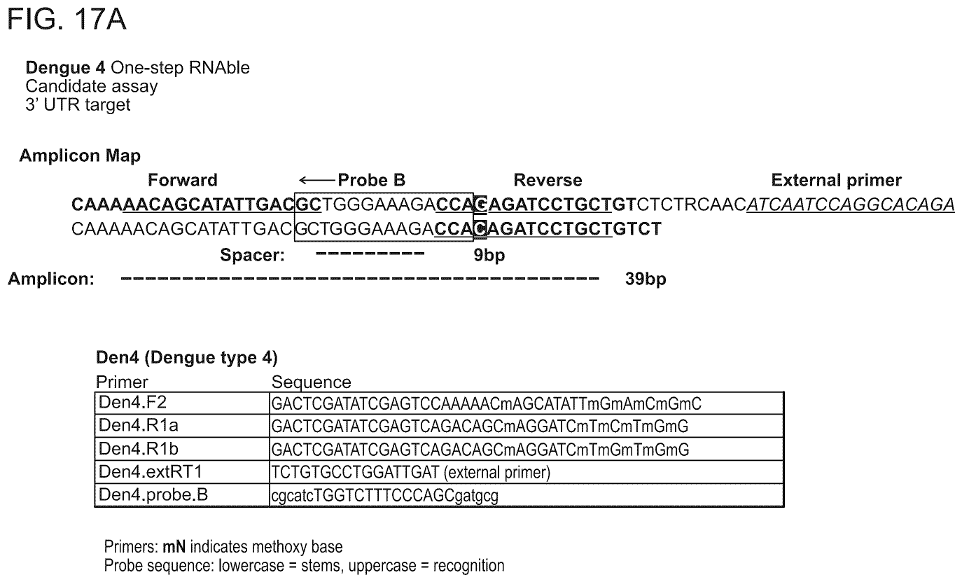

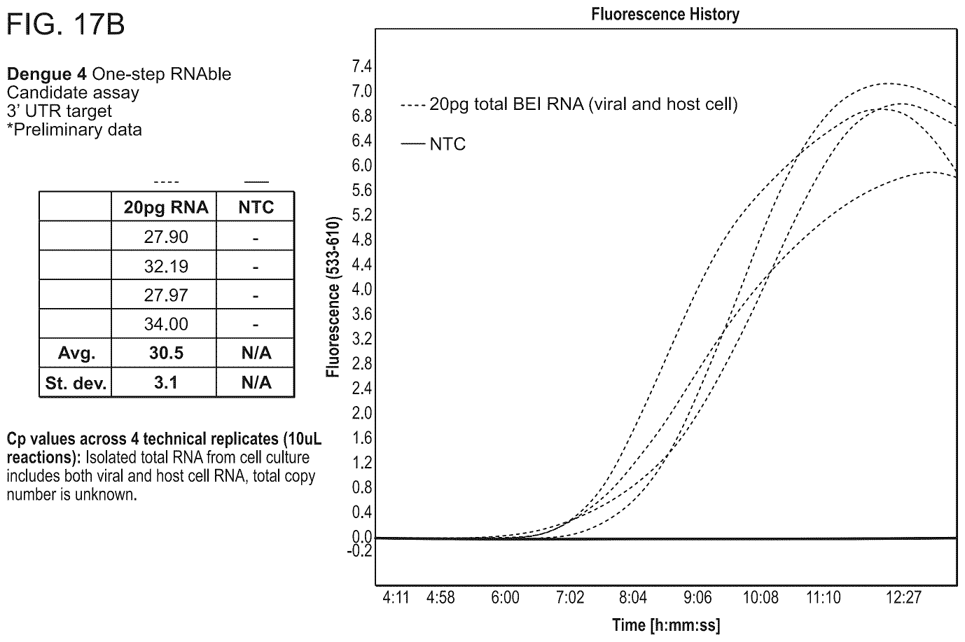

FIG. 16A is a map of the amplicon showing the locations of the sequences used. The sequence of the external primer was specific to HIV subtype C (for the purified RNA sample used). The 1-step HIV RNAble® assay was designed with two sets of forward and reverse primers to account for sequence variations in the population. In the sequences above, GAGTC is the nicking enzyme recognition site.

The 1-step reactions contained a final concentration of 9 nM forward primer (1:1 mix of Hgag.F2a+Hgag.F2b) and a final concentration of 91 nM reverse primer (1:1 mix of Hgag.R1a+Hgag.R1b) in a primer ratio of about 1:10 forward to reverse primers. Final concentrations of 200 nM probe and 100 nM external primer were used. In addition, the 1 step reactions contained a final concentration of 1×Run Buffer, comprising Tris, K⁺, and Mg²⁺; Guanidinium thiocyanate (GITC); dNTPs; 0.4 U/μl BST polymerase; and 0.3 U/μl Nt.BSTnbi nicking enzyme. In addition to these components; the 1 step reactions contained 0.2 U/μl of AMV Reverse Transcriptase High Spec Activity XL (Life Sciences Advanced Technologies) and 0.5 U/μl of an RNAse inhibitor (Superasin; Thermo Fisher). All water used was nuclease free.

Reactions were run using real-time detection of calfluor red 610 beacon signal (Abs 590 nm/Em 610 nm). 1-Step reactions were carried out at 56° C. for 20 minutes using 125, 250, 500, and 1000 copies of HIV RNA. Specific detection of HIV RNA at all copy numbers was demonstrated in the 1-step HIV RNAble® assay (FIG. 16B).

### Example 5: Detection of Dengue Virus Type 4 (DENV-4) in a Biological Sample

In one working example, dengue virus type 4 (DENV-4) was detected in a 1-step RNAble® assay targeting a 3′ UTR sequence. Total RNA was isolated from cell culture, which included both viral and host cell RNA, and used in the 1-step DENV-4 RNAble® assay. Thus, total copy number was unknown. The following primers and probe sequences were used:

Den4 (Dengue type 4)

| Primer | Sequence | SEQ ID NO |
|---|---|---|
| Den4.F2 | GACTCGATATCGAGTCCAAAAACmAGCATATTmGm | SEQ ID NO: 11 |
| | AmCmGmC | |
| Den4.R1a | GACTCGATATCGAGTCAGACAGCmAGGATCmTmCm | SEQ ID NO: 12 |
| | TmGmG | |
| Den4.R1b | GACTCGATATCGAGTCAGACAGCmAGGATCmTmGm | SEQ ID NO: 13 |
| | TmGmG | |
| Den4.extRT1 | TCTGTGCCTGGATTGAT (external primer) | SEQ ID NO: 44 |
| Den4.probe.B | cgcatcTGGTCTTTCCCAGCgatgcg | SEQ ID NO: 14 |

Primers: mN indicates methoxy base
Probe sequence: lowercase = stems, uppercase = recognition

FIG. 17A is a map of the amplicon showing the locations of the sequences used. The 1-step DENV-4 RNAble® assay was designed with two sets of reverse primers to account for sequence variations in the population. In the sequences above, GAGTC is the nicking enzyme recognition site.

The 1-step reactions contained a final concentration of 83 nM forward primer and a final concentration of 17 nM reverse primer (1:1 mix of Den4.R1a+Den4.R1b) in a primer ratio of about 5:1 forward to reverse primers. Final concentrations of 200 nM probe and 100 nM external primer were used. In addition, the 1 step reactions contained a final concentration of 1× Run Buffer, comprising Tris, K⁺, and Mg²⁺; dNTPs; 0.4 U/μl BST polymerase; and 0.3 U/μl Nt.BSTnbi nicking enzyme. In addition to these components; the 1 step reactions contained 0.2 U/μl of AMV Reverse Transcriptase High Spec Activity XL (Life Sciences Advanced Technologies) and 0.5 U/μl of an RNAse inhibitor (Superasin; Thermo Fisher). All water used was nuclease free.

Reactions were run using real-time detection of calfluor red 610 beacon signal (Abs 590 nm/Em 610 nm). 1-Step reactions were carried out at 56° C. for 20 minutes using 20 pg total RNA. Specific detection of Dengue 4 RNA was demonstrated in the 1-step DENV-4 RNAble® assay (FIG. 17B).

### Example 6: Detection of Influenza B in a Biological Sample

In one working example, influenza B was detected in a 1-step RNAble® assay targeting an influenza Segment 7 sequence. Total RNA was isolated from cell culture, which included both viral and host cell RNA, and used in the 1-step influenza B RNAble® assay. Thus, total copy number was unknown. The following primers and probe sequences were used:

FluB (influenza B)

| Primer | Sequence | SEQ ID NO |
|---|---|---|
| FluB.F2a | GACTCGATATCGAGTCAAATGCAmGATGGTCTCmA | SEQ ID NO: 15 |
| | mGmCmTmA | |
| FluB.F2b | GACTCGATATCGAGTCAAATGCAmAATGGTCTCmA | SEQ ID NO: 16 |
| | mGmCmTmA | |
| FluB.F2c | GACTCGATATCGAGTCAAATGCAmGATGGTTTCmA | SEQ ID NO: 17 |
| | mGmCmTmA | |
| FluB.R3a | GACTCGATATCGAGTCCTCCTTTmTCCCATTCCAT | SEQ ID NO: 18 |
| | mTmCmAmTmT | |
| FluB.R3b | GACTCGATATCGAGTCCTCCCTTmTCCCATTCCAT | SEQ ID NO: 19 |
| | mTmCmAmTmT | |
| FluB.R3c | GACTCGATATCGAGTCCTCCTTTmCCCCATTCCAT | SEQ ID NO: 20 |
| | mTmCmAmTmT | |
| FluB.extRT1a | TTTTGGACGTCTTCTCC (external primer) | SEQ ID NO: 45 |
| FluB.extRT1b | TTTTGAACGTCTTCTCC (external primer) | SEQ ID NO: 46 |
| FluB.probe.T | gccaaGCTATGAACACAGCAAActtggc | SEQ ID NO: 21 |

Primers: mN indicates methoxy base
Probe sequence: lowercase = stems, uppercase = recognition

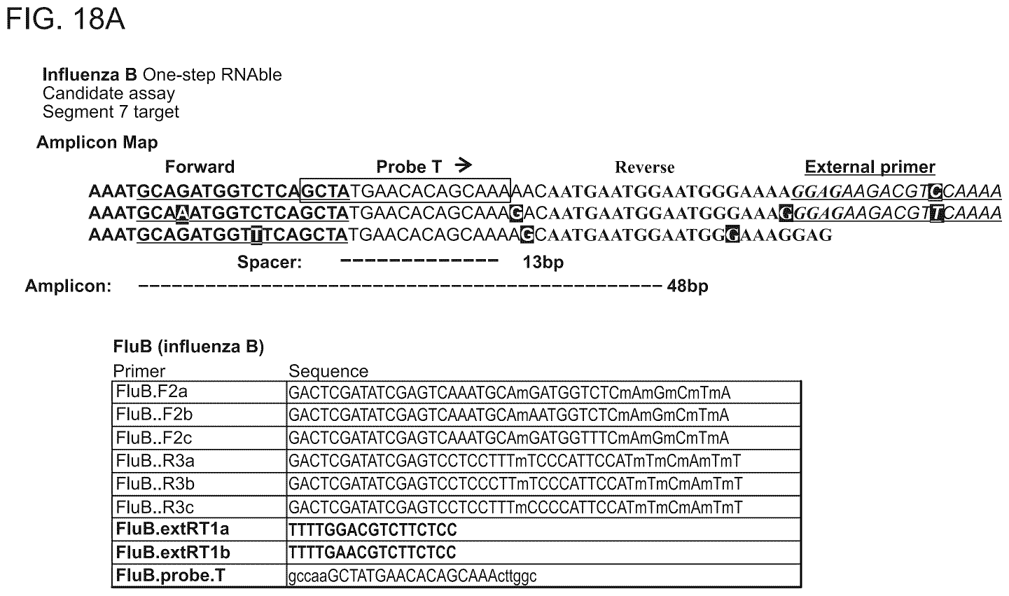

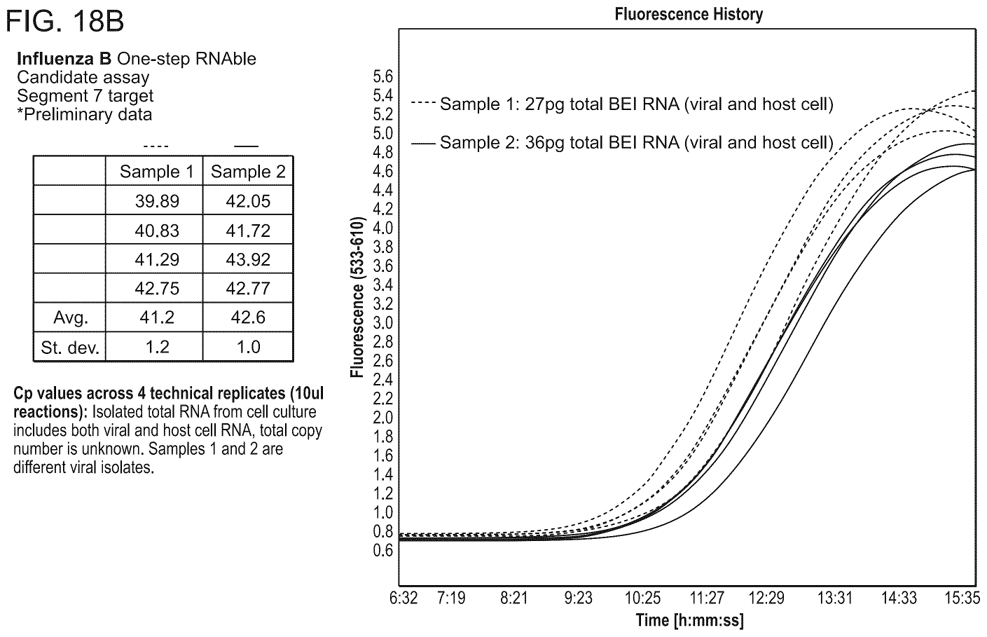

FIG. 18A is a map of the amplicon showing the locations of the sequences used. The 1-step Influenza B RNAble® assay was designed with three sets of forward and reverse primers and two external primers to account for sequence variations in the population. In the sequences above, GAGTC is the nicking enzyme recognition site.

The 1-step reactions contained a final concentration of 9 nM forward primer (1:1:1 mix of FluB.F2a+FluB.F2b+FluB.F2c) and a final concentration of 91 nM reverse primer (1:1:1 mix of F1uB.R3a+F1uB.R3b+F1uB.R3c) in a primer ratio of about 1:10 forward to reverse primers. Final concentrations of 200 nM probe and 100 nM external primer were used.

In addition, the 1 step reactions contained a final concentration of 1× Run Buffer, comprising Tris, K⁺, and Mg²⁺; dNTPs; 0.4 U/μl BST polymerase; and 0.3 U/μl Nt.BSTnbi nicking enzyme. In addition to these components; the 1 step reactions contained 0.2 U/μl of AMV Reverse Transcriptase High Spec Activity XL (Life Sciences Advanced Technologies) and 0.5 U/μl of an RNAse inhibitor (Superasin; Thermo Fisher). All water used was nuclease free.

Reactions were run using real-time detection of calfluor red 610 beacon signal (Abs 590 nm/Em 610 nm). 1-Step reactions were carried out at 56° C. for 20 minutes using total RNA from different isolates. Specific detection of influenza B in all isolates was demonstrated in the 1-step influenza B RNAble® assay (FIG. 18B).

### Example 7: Detection of Bovine Viral Diarrhea Virus Genotype 1 (BVDV1) in a Biological Sample

In one working example, Bovine Viral Diarrhea Virus Genotype 1 (BVDV1) was detected in a 1-step RNAble® assay targeting an influenza Segment 7 sequence. Total RNA was isolated from cell culture, which included both viral and host cell RNA, and used in the 1-step BVDV1 RNAble® assay. Thus, total copy number was unknown. The following primers and probe sequences were used:

Bovine Viral Diarrhea Virus Type 1 (BVDV1)

| Primer | Sequence | SEQ ID NO |
|---|---|---|
| BVDV1.F1a | GACTCGATATCGAGTCGGCCCACmTGTATTGCT | SEQ ID NO: 22 |
| | mAmCmTmGmAmAmA | |
| BVDV1.F1b | GACTCGATATCGAGTCGGCCCACmTGCACTGCT | SEQ ID NO: 23 |
| | mAmCmTmAmAmAmA | |
| BVDV1.R1 | GACTCGATATCGAGTCTGTGATCmAACTCCmAm | SEQ ID NO: 24 |
| | TmGmTmGmCmC | |
| BVDV1.RT1v | TATGTTTTGTATAAAAGTTCATTTG | SEQ ID NO: 47 |
| | (external primer) | |
| BVDV1.ProbeT | cgctacATCTCTGCTGTACATGgtagcg | SEQ ID NO: 25 |

Primers: mN indicates methoxy base
Probe sequence: lowercase = stems, uppercase = recognition

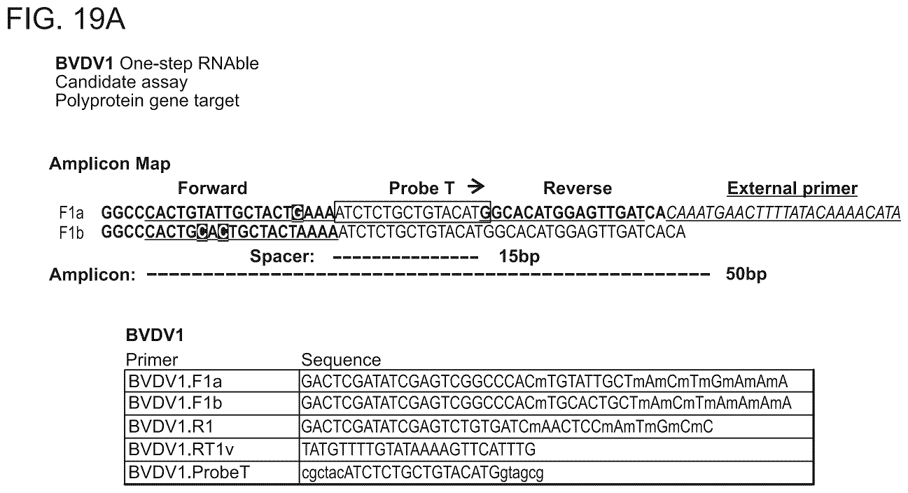

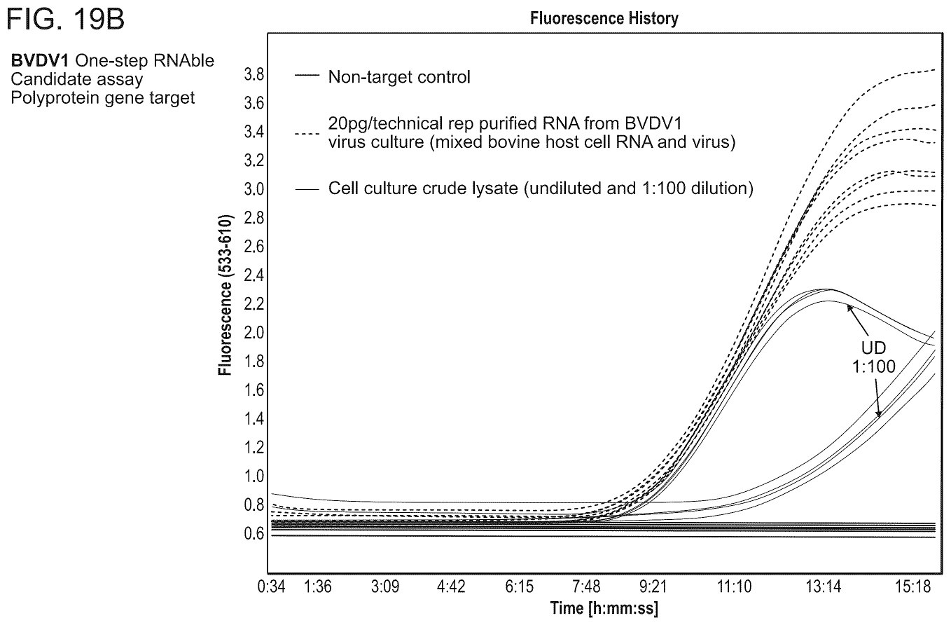

FIG. 19A is a map of the amplicon showing the locations of the sequences used. The 1-step BVDV1 RNAble® assay was designed with two sets of forward primers to account for sequence variations in the population. In the sequences above, GAGTC is the nicking enzyme recognition site.

The 1-step reactions contained a final concentration of 9 nM forward primer (1:1 mix of BVDV1.F1a+BVDV1.F1b) and a final concentration of 91 nM reverse primer in a primer ratio of about 1:10 forward to reverse primers. Final concentrations of 200 nM probe and 100 nM external primer were used. In addition, the 1 step reactions contained a final concentration of 1× Run Buffer, comprising Tris, K⁺, and Mg²⁺; dNTPs; 0.4 U/μl BST polymerase; and 0.3 U/μl Nt.BSTnbi nicking enzyme. In addition to these components; the 1 step reactions contained 0.2 U/μl of AMV Reverse Transcriptase High Spec Activity XL (Life Sciences Advanced Technologies) and 0.5 U/μl of an RNAse inhibitor (Superasin; Thermo Fisher). All water used was nuclease free.

Reactions were run using real-time detection of calfluor red 610 beacon signal (Abs 590 nm/Em 610 nm). 1-Step reactions were carried out at 56° C. for 20 minutes using purified RNA (20 pg/technical replicate) from BVDV1 virus culture (mixed bovine host cell RNA and virus) and cell culture crude lysate (undiluted and 1:100 dilution). Specific detection of BVDV1 virus in all samples was demonstrated in the 1-step BVDV1 RNAble® assay (FIG. 19B).

### Example 8: Molecular Beacon Recognition of Ebola Strains

### Exemplary Beacon BLAST Alignment Output and Strains List:

Zaire ebolavirus isolate. Hapiens-tc/COD/1976Nambuku-Ecran complete genome
Sequence ID: gb[KM655246.1] Length: 18797 Number of Matches: 5

Range 1: 6513 to 6528 GenBank Graphics
▾ Next Match ▴ Previous Match

| Score | Expect | Identities | Gaps | Strand |
|---|---|---|---|---|
| 29.4 | 0.017 | 15/16(94%) | 0/16(0%) | Plus/Plus |
| bits(14) | | | | |

| | | | |
|---|---|---|---|
| Query | 1 | ACGACTTTYGCTGAAG | 16 |
| | | \|\|\|\|\|\|\|\| \|\|\|\|\|\|\| | |
| Sbjct | 6513 | ACGACTTTCGCTGAAG | 6528 |

Query 1 (SEQ ID NO: 48)
Subject (SEQ ID NO: 49)

### Beacon Alone Strains List:

Zaire ebolavirus isolate H. sapiens-tc/COD/1976/Yambuku-Ecran, complete genome

| | | | | | |
|---|---|---|---|---|---|
| 29.4 | 89.2 | 100% | 0.017 | 94% | KM655246.1 |

Zaire ebolavirus isolate H. sapiens-wt/GIN/2014/Gueckedou-C05, complete genome

| | | | | | |
|---|---|---|---|---|---|
| 29.4 | 104 | 100% | 0.017 | 94% | KJ660348.2 |

Zaire ebolavirus isolate H. sapiens-wt/GIN/2014/Gueckedou-C07, complete genome

| | | | | | |
|---|---|---|---|---|---|
| 29.4 | 104 | 100% | 0.017 | 94% | KJ660347.2 |

Zaire ebolavirus isolate H. sapiens-wt/GIN/2014/Kissidougou-C15, complete genome

| | | | | | |
|---|---|---|---|---|---|
| 29.4 | 104 | 100% | 0.017 | 94% | KJ660346.2 |

Zaire ebolavirus isolate Ebola virus H. sapiens-wt/SLE/2014/ManoRiver-NM042.3, partial genome

| | | | | | |
|---|---|---|---|---|---|
| 29.4 | 104 | 100% | 0.017 | 94% | KM233118.1 |

Zaire ebolavirus isolate Ebola virus H. sapiens-wt/SLE/2014/ManoRiver-NM042.2, partial genome

| | | | | | |
|---|---|---|---|---|---|
| 29.4 | 104 | 100% | 0.017 | 94% | KM233117.1 |

Zaire ebolavirus isolate Ebola virus H. sapiens-wt/SLE/2014/ManoRiver-NM042.1, partial genome

| | | | | | |
|---|---|---|---|---|---|
| 29.4 | 104 | 100% | 0.017 | 94% | KM233116.1 |

Zaire ebolavirus isolate Ebola virus H. sapiens-wt/SLE/2014/ManoRiver-G3857, partial genome

| | | | | | |
|---|---|---|---|---|---|
| 29.4 | 104 | 100% | 0.017 | 94% | KM233115.1 |

Zaire ebolavirus isolate Ebola virus H. sapiens-wt/SLE/2014/ManoRiver-G3856.3, partial genome

| | | | | | |
|---|---|---|---|---|---|
| 29.4 | 104 | 100% | 0.017 | 94% | KM233114.1 |

Zaire ebolavirus isolate Ebola virus H. sapiens-wt/SLE/2014/ManoRiver-G3856.1, partial genome

| | | | | | |
|---|---|---|---|---|---|
| 29.4 | 104 | 100% | 0.017 | 94% | KM233113.1 |

Zaire ebolavirus isolate Ebola virus H. sapiens-wt/SLE/2014/ManoRiver-G3851, partial genome

| | | | | | |
|---|---|---|---|---|---|
| 29.4 | 104 | 100% | 0.017 | 94% | KM233112.1 |

Zaire ebolavirus isolate Ebola virus H. sapiens-wt/SLE/2014/ManoRiver-G3850, partial genome

| | | | | | |
|---|---|---|---|---|---|
| 29.4 | 104 | 100% | 0.017 | 94% | KM233111.1 |

Zaire ebolavirus isolate Ebola virus H. sapiens-wt/SLE/2014/ManoRiver-G3848, partial genome

| | | | | | |
|---|---|---|---|---|---|
| 29.4 | 104 | 100% | 0.017 | 94% | KM233110.1 |

Zaire ebolavirus isolate Ebola virus H. sapiens-wt/SLE/2014/ManoRiver-G3846, partial genome

| | | | | | |
|---|---|---|---|---|---|
| 29.4 | 104 | 100% | 0.017 | 94% | KM233109.1 |

Zaire ebolavirus isolate Ebola virus H. sapiens-wt/SLE/2014/ManoRiver-G3841, partial genome

| | | | | | |
|---|---|---|---|---|---|
| 29.4 | 104 | 100% | 0.017 | 94% | KM233107.1 |

Zaire ebolavirus isolate Ebola virus H. sapiens-wt/SLE/2014/ManoRiver-G3840, partial genome

| | | | | | |
|---|---|---|---|---|---|
| 29.4 | 104 | 100% | 0.017 | 94% | KM233106.1 |

Zaire ebolavirus isolate Ebola virus H. sapiens-wt/SLE/2014/ManoRiver-G3838, partial genome

| | | | | | |
|---|---|---|---|---|---|
| 29.4 | 104 | 100% | 0.017 | 94% | KM233105.1 |

Zaire ebolavirus isolate Ebola virus H. sapiens-wt/SLE/2014/ManoRiver-G3834, partial genome

| | | | | | |
|---|---|---|---|---|---|
| 29.4 | 104 | 100% | 0.017 | 94% | KM233104.1 |

Zaire ebolavirus isolate Ebola virus H. sapiens-wt/SLE/2014/ManoRiver-G3831, partial genome

| | | | | | |
|---|---|---|---|---|---|
| 29.4 | 104 | 100% | 0.017 | 94% | KM233103.1 |

Zaire ebolavirus isolate Ebola virus H. sapiens-wt/SLE/2014/ManoRiver-G3829, partial genome

| | | | | | |
|---|---|---|---|---|---|
| 29.4 | 104 | 100% | 0.017 | 94% | KM233102.1 |

Zaire ebolavirus isolate Ebola virus H. sapiens-wt/SLE/2014/ManoRiver-G3827, partial genome

| | | | | | |
|---|---|---|---|---|---|
| 29.4 | 104 | 100% | 0.017 | 94% | KM233101.1 |

Zaire ebolavirus isolate Ebola virus H. sapiens-wt/SLE/2014/ManoRiver-G3826, partial genome

| | | | | | |
|---|---|---|---|---|---|
| 29.4 | 104 | 100% | 0.017 | 94% | KM233100.1 |

Zaire ebolavirus isolate Ebola virus H. sapiens-wt/SLE/2014/ManoRiver-G3825.2, partial genome

| | | | | | |
|---|---|---|---|---|---|
| 29.4 | 104 | 100% | 0.017 | 94% | KM233099.1 |

Zaire ebolavirus isolate Ebola virus H. sapiens-wt/SLE/2014/ManoRiver-G3825.1, partial genome

| | | | | | |
|---|---|---|---|---|---|
| 29.4 | 104 | 100% | 0.017 | 94% | KM233098.1 |

Zaire ebolavirus isolate Ebola virus H. sapiens-wt/SLE/2014/ManoRiver-G3823, partial genome

| | | | | | |
|---|---|---|---|---|---|
| 29.4 | 104 | 100% | 0.017 | 94% | KM233097.1 |

Zaire ebolavirus isolate Ebola virus H. sapiens-wt/SLE/2014/ManoRiver-G3822, partial genome

| | | | | | |
|---|---|---|---|---|---|
| 29.4 | 104 | 100% | 0.017 | 94% | KM233096.1 |

Zaire ebolavirus isolate Ebola virus H. sapiens-wt/SLE/2014/ManoRiver-G3821, partial genome

| | | | | | |
|---|---|---|---|---|---|
| 29.4 | 104 | 100% | 0.017 | 94% | KM233095.1 |

Zaire ebolavirus isolate Ebola virus H. sapiens-wt/SLE/2014/ManoRiver-G3819, partial genome

| | | | | | |
|---|---|---|---|---|---|
| 29.4 | 104 | 100% | 0.017 | 94% | KM233093.1 |

Zaire ebolavirus isolate Ebola virus H. sapiens-wt/SLE/2014/ManoRiver-G3818, partial genome

| | | | | | |
|---|---|---|---|---|---|
| 29.4 | 104 | 100% | 0.017 | 94% | KM233092.1 |

Zaire ebolavirus isolate Ebola virus H. sapiens-wt/SLE/2014/ManoRiver-G3817, partial genome

| | | | | | |
|---|---|---|---|---|---|
| 29.4 | 104 | 100% | 0.017 | 94% | KM233091.1 |

Zaire ebolavirus isolate Ebola virus H. sapiens-wt/SLE/2014/ManoRiver-G3816, partial genome

| | | | | | |
|---|---|---|---|---|---|
| 29.4 | 104 | 100% | 0.017 | 94% | KM233090.1 |

Zaire ebolavirus isolate Ebola virus H. sapiens-wt/SLE/2014/ManoRiver-G3814, partial genome

| | | | | | |
|---|---|---|---|---|---|
| 29.4 | 104 | 100% | 0.017 | 94% | KM233089.1 |

Zaire ebolavirus isolate Ebola virus H. sapiens-wt/SLE/2014/ManoRiver-G3810.2, partial genome

| | | | | | |
|---|---|---|---|---|---|
| 29.4 | 104 | 100% | 0.017 | 94% | KM233088.1 |

Zaire ebolavirus isolate Ebola virus H. sapiens-wt/SLE/2014/ManoRiver-G3810.1, partial genome

| | | | | | |
|---|---|---|---|---|---|
| 29.4 | 104 | 100% | 0.017 | 94% | KM233087.1 |

Zaire ebolavirus isolate Ebola virus H. sapiens-wt/SLE/2014/ManoRiver-G3809, partial genome

| | | | | | |
|---|---|---|---|---|---|
| 29.4 | 104 | 100% | 0.017 | 94% | KM233086.1 |

Zaire ebolavirus isolate Ebola virus H. sapiens-wt/SLE/2014/ManoRiver-G3808, partial genome

| | | | | | |
|---|---|---|---|---|---|
| 29.4 | 104 | 100% | 0.017 | 94% | KM233085.1 |

Zaire ebolavirus isolate Ebola virus H. sapiens-wt/SLE/2014/ManoRiver-G3807, partial genome

| | | | | | |
|---|---|---|---|---|---|
| 29.4 | 104 | 100% | 0.017 | 94% | KM233084.1 |

Zaire ebolavirus isolate Ebola virus H. sapiens-wt/SLE/2014/ManoRiver-G3805.2, partial genome

| | | | | | |
|---|---|---|---|---|---|
| 29.4 | 104 | 100% | 0.017 | 94% | KM233083.1 |

Zaire ebolavirus isolate Ebola virus H. sapiens-wt/SLE/2014/ManoRiver-G3805.1, partial genome

| | | | | | |
|---|---|---|---|---|---|
| 29.4 | 104 | 100% | 0.017 | 94% | KM233082.1 |

Zaire ebolavirus isolate Ebola virus H. sapiens-wt/SLE/2014/ManoRiver-G3800, partial genome

| | | | | | |
|---|---|---|---|---|---|
| 29.4 | 104 | 100% | 0.017 | 94% | KM233081.1 |

Zaire ebolavirus isolate Ebola virus H. sapiens-wt/SLE/2014/ManoRiver-G3799, partial genome

| | | | | | |
|---|---|---|---|---|---|
| 29.4 | 104 | 100% | 0.017 | 94% | KM233080.1 |

Zaire ebolavirus isolate Ebola virus H. sapiens-wt/SLE/2014/ManoRiver-G3798, partial genome

| | | | | | |
|---|---|---|---|---|---|
| 29.4 | 104 | 100% | 0.017 | 94% | KM233079.1 |

Zaire ebolavirus isolate Ebola virus H. sapiens-wt/SLE/2014/ManoRiver-G3795, partial genome

| | | | | | |
|---|---|---|---|---|---|
| 29.4 | 104 | 100% | 0.017 | 94% | KM233077.1 |

Zaire ebolavirus isolate Ebola virus H. sapiens-wt/SLE/2014/ManoRiver-G3789.1, partial genome

| | | | | | |
|---|---|---|---|---|---|
| 29.4 | 104 | 100% | 0.017 | 94% | KM233076.1 |

Zaire ebolavirus isolate Ebola virus H. sapiens-wt/SLE/2014/ManoRiver-G3788, partial genome

| | | | | | |
|---|---|---|---|---|---|
| 29.4 | 104 | 100% | 0.017 | 94% | KM233075.1 |

Zaire ebolavirus isolate Ebola virus H. sapiens-wt/SLE/2014/ManoRiver-G3787, partial genome

| | | | | | |
|---|---|---|---|---|---|
| 29.4 | 104 | 100% | 0.017 | 94% | KM233074.1 |

Zaire ebolavirus isolate Ebola virus H. sapiens-wt/SLE/2014/ManoRiver-G3786, partial genome

| | | | | | |
|---|---|---|---|---|---|
| 29.4 | 104 | 100% | 0.017 | 94% | KM233073.1 |

Zaire ebolavirus isolate Ebola virus H. sapiens-wt/SLE/2014/ManoRiver-G3782, partial genome

| | | | | | |
|---|---|---|---|---|---|
| 29.4 | 104 | 100% | 0.017 | 94% | KM233072.1 |

Zaire ebolavirus isolate Ebola virus H. sapiens-wt/SLE/2014/ManoRiver-G3771, partial genome

| | | | | | |
|---|---|---|---|---|---|
| 29.4 | 104 | 100% | 0.017 | 94% | KM233071.1 |

Zaire ebolavirus isolate Ebola virus H. sapiens-wt/SLE/2014/ManoRiver-G3770.2, partial genome

| | | | | | |
|---|---|---|---|---|---|
| 29.4 | 104 | 100% | 0.017 | 94% | KM233070.1 |

Zaire ebolavirus isolate Ebola virus H. sapiens-wt/SLE/2014/ManoRiver-G3770.1, partial genome

| | | | | | |
|---|---|---|---|---|---|
| 29.4 | 104 | 100% | 0.017 | 94% | KM233069.1 |

Zaire ebolavirus isolate Ebola virus H. sapiens-wt/SLE/2014/ManoRiver-G3769.3, partial genome

| | | | | | |
|---|---|---|---|---|---|
| 29.4 | 104 | 100% | 0.017 | 94% | KM233067.1 |

Zaire ebolavirus isolate Ebola virus H. sapiens-wt/SLE/2014/ManoRiver-G3769.2, partial genome

| | | | | | |
|---|---|---|---|---|---|
| 29.4 | 104 | 100% | 0.017 | 94% | KM233066.1 |

Zaire ebolavirus isolate Ebola virus H. sapiens-wt/SLE/2014/ManoRiver-G3769.1, partial genome

| | | | | | |
|---|---|---|---|---|---|
| 29.4 | 104 | 100% | 0.017 | 94% | KM233065.1 |

Zaire ebolavirus isolate Ebola virus H. sapiens-wt/SLE/2014/ManoRiver-G3765.2, partial genome

| | | | | | |
|---|---|---|---|---|---|
| 29.4 | 104 | 100% | 0.017 | 94% | KM233064.1 |

Zaire ebolavirus isolate Ebola virus H. sapiens-wt/SLE/2014/ManoRiver-G3764, partial genome

| | | | | | |
|---|---|---|---|---|---|
| 29.4 | 104 | 100% | 0.017 | 94% | KM233063.1 |

Zaire ebolavirus isolate Ebola virus H. sapiens-wt/SLE/2014/ManoRiver-G3758, partial genome

| | | | | | |
|---|---|---|---|---|---|
| 29.4 | 104 | 100% | 0.017 | 94% | KM233062.1 |

Zaire ebolavirus isolate Ebola virus H. sapiens-wt/SLE/2014/ManoRiver-G3752, partial genome

| | | | | | |
|---|---|---|---|---|---|
| 29.4 | 104 | 100% | 0.017 | 94% | KM233061.1 |

Zaire ebolavirus isolate Ebola virus H. sapiens-wt/SLE/2014/ManoRiver-G3750.3, partial genome

| | | | | | |
|---|---|---|---|---|---|
| 29.4 | 104 | 100% | 0.017 | 94% | KM233060.1 |

Zaire ebolavirus isolate Ebola virus H. sapiens-wt/SLE/2014/ManoRiver-G3750.2, partial genome

| | | | | | |
|---|---|---|---|---|---|
| 29.4 | 104 | 100% | 0.017 | 94% | KM233059.1 |

Zaire ebolavirus isolate Ebola virus H. sapiens-wt/SLE/2014/ManoRiver-G3750.1, partial genome

| | | | | | |
|---|---|---|---|---|---|
| 29.4 | 104 | 100% | 0.017 | 94% | KM233058.1 |

Zaire ebolavirus isolate Ebola virus H. sapiens-wt/SLE/2014/ManoRiver-G3735.2, partial genome

| | | | | | |
|---|---|---|---|---|---|
| 29.4 | 104 | 100% | 0.017 | 94% | KM233057.1 |

Zaire ebolavirus isolate Ebola virus H. sapiens-wt/SLE/2014/ManoRiver-G3735.1, partial genome

| | | | | | |
|---|---|---|---|---|---|
| 29.4 | 104 | 100% | 0.017 | 94% | KM233056.1 |

Zaire ebolavirus isolate Ebola virus H. sapiens-wt/SLE/2014/ManoRiver-G3734.1, partial genome

| | | | | | |
|---|---|---|---|---|---|
| 29.4 | 104 | 100% | 0.017 | 94% | KM233055.1 |

Zaire ebolavirus isolate Ebola virus H. sapiens-wt/SLE/2014/ManoRiver-G3729, partial genome

| | | | | | |
|---|---|---|---|---|---|
| 29.4 | 104 | 100% | 0.017 | 94% | KM233054.1 |

Zaire ebolavirus isolate Ebola virus H. sapiens-wt/SLE/2014/ManoRiver-G3724, partial genome

| | | | | | |
|---|---|---|---|---|---|
| 29.4 | 104 | 100% | 0.017 | 94% | KM233053.1 |

Zaire ebolavirus isolate Ebola virus H. sapiens-wt/SLE/2014/ManoRiver-G3713.4, partial genome

| | | | | | |
|---|---|---|---|---|---|
| 29.4 | 104 | 100% | 0.017 | 94% | KM233052.1 |

Zaire ebolavirus isolate Ebola virus H. sapiens-wt/SLE/2014/ManoRiver-G3713.3, partial genome

| | | | | | |
|---|---|---|---|---|---|
| 29.4 | 104 | 100% | 0.017 | 94% | KM233051.1 |

Zaire ebolavirus isolate Ebola virus H. sapiens-wt/SLE/2014/ManoRiver-G3713.2, partial genome

| | | | | | |
|---|---|---|---|---|---|
| 29.4 | 104 | 100% | 0.017 | 94% | KM233050.1 |

Zaire ebolavirus isolate Ebola virus H. sapiens-wt/SLE/2014/ManoRiver-G3707, partial genome

| | | | | | |
|---|---|---|---|---|---|
| 29.4 | 104 | 100% | 0.017 | 94% | KM233049.1 |

Zaire ebolavirus isolate Ebola virus H. sapiens-wt/SLE/2014/ManoRiver-EM124.4, partial genome

| | | | | | |
|---|---|---|---|---|---|
| 29.4 | 104 | 100% | 0.017 | 94% | KM233048.1 |

Zaire ebolavirus isolate Ebola virus H. sapiens-wt/SLE/2014/ManoRiver-EM124.3, partial genome

| | | | | | |
|---|---|---|---|---|---|
| 29.4 | 104 | 100% | 0.017 | 94% | KM233047.1 |

Zaire ebolavirus isolate Ebola virus H. sapiens-wt/SLE/2014/ManoRiver-EM124.2, partial genome

| | | | | | |
|---|---|---|---|---|---|
| 29.4 | 104 | 100% | 0.017 | 94% | KM233046.1 |

Zaire ebolavirus isolate Ebola virus H. sapiens-wt/SLE/2014/ManoRiver-EM124.1, partial genome

| | | | | | |
|---|---|---|---|---|---|
| 29.4 | 104 | 100% | 0.017 | 94% | KM233045.1 |

Zaire ebolavirus isolate Ebola virus H. sapiens-wt/SLE/2014/ManoRiver-EM121, partial genome

| | | | | | |
|---|---|---|---|---|---|
| 29.4 | 104 | 100% | 0.017 | 94% | KM233044.1 |

Zaire ebolavirus isolate Ebola virus H. sapiens-wt/SLE/2014/ManoRiver-EM120, partial genome

| | | | | | |
|---|---|---|---|---|---|
| 29.4 | 104 | 100% | 0.017 | 94% | KM233043.1 |

Zaire ebolavirus isolate Ebola virus H. sapiens-wt/SLE/2014/ManoRiver-EM119, partial genome

| | | | | | |
|---|---|---|---|---|---|
| 29.4 | 104 | 100% | 0.017 | 94% | KM233042.1 |

Zaire ebolavirus isolate Ebola virus H. sapiens-wt/SLE/2014/ManoRiver-EM115, partial genome

| | | | | | |
|---|---|---|---|---|---|
| 29.4 | 104 | 100% | 0.017 | 94% | KM233041.1 |

Zaire ebolavirus isolate Ebola virus H. sapiens-wt/SLE/2014/ManoRiver-EM113, partial genome

| | | | | | |
|---|---|---|---|---|---|
| 29.4 | 104 | 100% | 0.017 | 94% | KM233040.1 |

Zaire ebolavirus isolate Ebola virus H. sapiens-wt/SLE/2014/ManoRiver-EM112, partial genome

| | | | | | |
|---|---|---|---|---|---|
| 29.4 | 104 | 100% | 0.017 | 94% | KM233039.1 |

Zaire ebolavirus isolate Ebola virus H. sapiens-wt/SLE/2014/ManoRiver-EM111, partial genome

| | | | | | |
|---|---|---|---|---|---|
| 29.4 | 104 | 100% | 0.017 | 94% | KM233038.1 |

Zaire ebolavirus isolate Ebola virus H. sapiens-wt/SLE/2014/ManoRiver-EM110, partial genome

| | | | | | |
|---|---|---|---|---|---|
| 29.4 | 104 | 100% | 0.017 | 94% | KM233037.1 |

Zaire ebolavirus isolate Ebola virus H. sapiens-wt/SLE/2014/ManoRiver-EM106, partial genome

| | | | | | |
|---|---|---|---|---|---|
| 29.4 | 104 | 100% | 0.017 | 94% | KM233036.1 |

Zaire ebolavirus isolate Ebola virus H. sapiens-wt/SLE/2014/ManoRiver-EM104, partial genome

| | | | | | |
|---|---|---|---|---|---|
| 29.4 | 104 | 100% | 0.017 | 94% | KM233035.1 |

Zaire ebolavirus isolate Ebola virus H. sapiens-wt/SLE/2014/ManoRiver-G3687.1, partial genome

| | | | | | |
|---|---|---|---|---|---|
| 29.4 | 104 | 100% | 0.017 | 94% | KM034563.1 |

Zaire ebolavirus isolate Ebola virus H. sapiens-wt/SLE/2014/ManoRiver-G3686.1, partial genome

| | | | | | |
|---|---|---|---|---|---|
| 29.4 | 104 | 100% | 0.017 | 94% | KM034562.1 |

Zaire ebolavirus isolate Ebola virus H. sapiens-wt/SLE/2014/ManoRiver-G3683.1, partial genome

| | | | | | |
|---|---|---|---|---|---|
| 29.4 | 104 | 100% | 0.017 | 94% | KM034561.1 |

Zaire ebolavirus isolate Ebola virus H. sapiens-wt/SLE/2014/ManoRiver-G3682.1, partial genome

| | | | | | |
|---|---|---|---|---|---|
| 29.4 | 104 | 100% | 0.017 | 94% | KM034560.1 |

Zaire ebolavirus isolate Ebola virus H. sapiens-wt/SLE/2014/ManoRiver-G3680.1, partial genome

| | | | | | |
|---|---|---|---|---|---|
| 29.4 | 104 | 100% | 0.017 | 94% | KM034559.1 |

Zaire ebolavirus isolate Ebola virus H. sapiens-wt/SLE/2014/ManoRiver-G3679.1, partial genome

| | | | | | |
|---|---|---|---|---|---|
| 29.4 | 104 | 100% | 0.017 | 94% | KM034558.1 |

Zaire ebolavirus isolate Ebola virus H. sapiens-wt/SLE/2014/ManoRiver-G3677.2, partial genome

| | | | | | |
|---|---|---|---|---|---|
| 29.4 | 104 | 100% | 0.017 | 94% | KM034557.1 |

Zaire ebolavirus isolate Ebola virus H. sapiens-wt/SLE/2014/ManoRiver-G3677.1, partial genome

| | | | | | |
|---|---|---|---|---|---|
| 29.4 | 104 | 100% | 0.017 | 94% | KM034556.1 |

Zaire ebolavirus isolate Ebola virus H. sapiens-wt/SLE/2014/ManoRiver-G3676.2, partial genome

| | | | | | |
|---|---|---|---|---|---|
| 29.4 | 104 | 100% | 0.017 | 94% | KM034555.1 |

Zaire ebolavirus isolate Ebola virus H. sapiens-wt/SLE/2014/ManoRiver-G3676.1, partial genome

| | | | | | |
|---|---|---|---|---|---|
| 29.4 | 104 | 100% | 0.017 | 94% | KM034554.1 |

Zaire ebolavirus isolate Ebola virus H. sapiens-wt/SLE/2014/ManoRiver-G3670.1, partial genome

| | | | | | |
|---|---|---|---|---|---|
| 29.4 | 104 | 100% | 0.017 | 94% | KM034553.1 |

Zaire ebolavirus isolate Ebola virus H. sapiens-wt/SLE/2014/ManoRiver-EM098, partial genome

| | | | | | |
|---|---|---|---|---|---|
| 29.4 | 104 | 100% | 0.017 | 94% | KM034552.1 |

Zaire ebolavirus isolate Ebola virus H. sapiens-wt/SLE/2014/ManoRiver-EM096, partial genome

| | | | | | |
|---|---|---|---|---|---|
| 29.4 | 104 | 100% | 0.017 | 94% | KM034551.1 |

Zaire ebolavirus isolate Ebola virus H. sapiens-wt/SLE/2014/ManoRiver-EM095, partial genome

| | | | | | |
|---|---|---|---|---|---|
| 29.4 | 104 | 100% | 0.017 | 94% | KM034550.1 |

Zaire ebolavirus isolate H. sapiens-wt/SLE/2014/ManoRiver-EM095B, partial genome

| | | | | | |
|---|---|---|---|---|---|
| 29.4 | 104 | 100% | 0.017 | 94% | KM034549.1 |

Mutant Zaire ebolavirus, complete sequence

| | | | | | |
|---|---|---|---|---|---|
| 29.4 | 89.2 | 100% | 0.017 | 94% | KF827427.1 |

### Amplicon Similarity Analysis Across Ebola Strains

### Exemplary BLAST Alignment Output and Strains List:

Zaire ebolavirus: isolate H. sapiens-tc/COD/1976/Yarmbuku-Ecran. complete genome
Sequence ID: gb[KM55245.1] Length: 18797 Number of Matches: 1

Range 1: 6492 to 6547 GenBank Graphics ▾ Next Match ▴ Previous Match

| Score | Expect | Identities | Gaps | Strand |
|---|---|---|---|---|
| 102 | 1e−23 | 56/56(100%) | 0/56(0%) | Plus/Plus |
| bits (112) | | | | |

| | | | |
|---|---|---|---|
| Query | 1 | TCCACAGTTATCTACCGAGGAACGACTTTCGCTGAAGGTGTCGTTGCATTTCTGAT | 56 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct | 6492 | TCCACAGTTATCTACCGAGGAACGACTTTCGCTGAAGGTGTCGTTGCATTTCTGAT | 6547 |

(SEQ ID NO: 50)

### Strains List:

Zaire ebolavirus isolate H. sapiens-tc/COD/1976/Yambuku-Ecran, complete genome

| | | | | | |
|---|---|---|---|---|---|
| 102 | 102 | 100% | 1e-23 | 100% | KM655246.1 |

Zaire ebolavirus isolate H. sapiens-wt/GIN/2014/Gueckedou-C05, complete genome

| | | | | | |
|---|---|---|---|---|---|
| 102 | 102 | 100% | 1e-23 | 100% | KJ660348.2 |

Zaire ebolavirus isolate H. sapiens-wt/GIN/2014/Gueckedou-C07, complete genome

| | | | | | |
|---|---|---|---|---|---|
| 102 | 102 | 100% | 1e-23 | 100% | KJ660347.2 |

Zaire ebolavirus isolate H. sapiens-wt/GIN/2014/Kissidougou-C15, complete genome

| | | | | | |
|---|---|---|---|---|---|
| 102 | 102 | 100% | 1e-23 | 100% | KJ660346.2 |

Zaire ebolavirus isolate Ebola virus H. sapiens-wt/SLE/2014/ManoRiver-NM042.3, partial genome

| | | | | | |
|---|---|---|---|---|---|
| 102 | 102 | 100% | 1e-23 | 100% | KM233118.1 |

Zaire ebolavirus isolate Ebola virus H. sapiens-wt/SLE/2014/ManoRiver-NM042.2, partial genome

| | | | | | |
|---|---|---|---|---|---|
| 102 | 102 | 100% | 1e-23 | 100% | KM233117.1 |

Zaire ebolavirus isolate Ebola virus H. sapiens-wt/SLE/2014/ManoRiver-NM042.1, partial genome

| | | | | | |
|---|---|---|---|---|---|
| 102 | 102 | 100% | 1e-23 | 100% | KM233116.1 |

Zaire ebolavirus isolate Ebola virus H. sapiens-wt/SLE/2014/ManoRiver-G3857, partial genome

| | | | | | |
|---|---|---|---|---|---|
| 102 | 102 | 100% | 1e-23 | 100% | KM233115.1 |

Zaire ebolavirus isolate Ebola virus H. sapiens-wt/SLE/2014/ManoRiver-G3856.3, partial genome

| | | | | | |
|---|---|---|---|---|---|
| 102 | 102 | 100% | 1e-23 | 100% | KM233114.1 |

Zaire ebolavirus isolate Ebola virus H. sapiens-wt/SLE/2014/ManoRiver-G3856.1, partial genome

| | | | | | |
|---|---|---|---|---|---|
| 102 | 102 | 100% | 1e-23 | 100% | KM233113.1 |

Zaire ebolavirus isolate Ebola virus H. sapiens-wt/SLE/2014/ManoRiver-G3851, partial genome

| | | | | | |
|---|---|---|---|---|---|
| 102 | 102 | 100% | 1e-23 | 100% | KM233112.1 |

Zaire ebolavirus isolate Ebola virus H. sapiens-wt/SLE/2014/ManoRiver-G3850, partial genome

| | | | | | |
|---|---|---|---|---|---|
| 102 | 102 | 100% | 1e-23 | 100% | KM233111.1 |

Zaire ebolavirus isolate Ebola virus H. sapiens-wt/SLE/2014/ManoRiver-G3848, partial genome

| | | | | | |
|---|---|---|---|---|---|
| 102 | 102 | 100% | 1e-23 | 100% | KM233110.1 |

Zaire ebolavirus isolate Ebola virus H. sapiens-wt/SLE/2014/ManoRiver-G3846, partial genome

| | | | | | |
|---|---|---|---|---|---|
| 102 | 102 | 100% | 1e-23 | 100% | KM233109.1 |

Zaire ebolavirus isolate Ebola virus H. sapiens-wt/SLE/2014/ManoRiver-G3841, partial genome

| | | | | | |
|---|---|---|---|---|---|
| 102 | 102 | 100% | 1e-23 | 100% | KM233107.1 |

Zaire ebolavirus isolate Ebola virus H. sapiens-wt/SLE/2014/ManoRiver-G3840, partial genome

| | | | | | |
|---|---|---|---|---|---|
| 102 | 102 | 100% | 1e-23 | 100% | KM233106.1 |

Zaire ebolavirus isolate Ebola virus H. sapiens-wt/SLE/2014/ManoRiver-G3838, partial genome

| | | | | | |
|---|---|---|---|---|---|
| 102 | 102 | 100% | 1e-23 | 100% | KM233105.1 |

Zaire ebolavirus isolate Ebola virus H. sapiens-wt/SLE/2014/ManoRiver-G3834, partial genome

| | | | | | |
|---|---|---|---|---|---|
| 102 | 102 | 100% | 1e-23 | 100% | KM233104.1 |

Zaire ebolavirus isolate Ebola virus H. sapiens-wt/SLE/2014/ManoRiver-G3831, partial genome

| | | | | | |
|---|---|---|---|---|---|
| 102 | 102 | 100% | 1e-23 | 100% | KM233103.1 |

Zaire ebolavirus isolate Ebola virus H. sapiens-wt/SLE/2014/ManoRiver-G3829, partial genome

| | | | | | |
|---|---|---|---|---|---|
| 102 | 102 | 100% | 1e-23 | 100% | KM233102.1 |

Zaire ebolavirus isolate Ebola virus H. sapiens-wt/SLE/2014/ManoRiver-G3827, partial genome

| | | | | | |
|---|---|---|---|---|---|
| 102 | 102 | 100% | 1e-23 | 100% | KM233101.1 |

Zaire ebolavirus isolate Ebola virus H. sapiens-wt/SLE/2014/ManoRiver-G3826, partial genome

| | | | | | |
|---|---|---|---|---|---|
| 102 | 102 | 100% | 1e-23 | 100% | KM233100.1 |

Zaire ebolavirus isolate Ebola virus H. sapiens-wt/SLE/2014/ManoRiver-G3825.2, partial genome

| | | | | | |
|---|---|---|---|---|---|
| 102 | 102 | 100% | 1e-23 | 100% | KM233099.1 |

Zaire ebolavirus isolate Ebola virus H. sapiens-wt/SLE/2014/ManoRiver-G3825.1, partial genome

| | | | | | |
|---|---|---|---|---|---|
| 102 | 102 | 100% | 1e-23 | 100% | KM233098.1 |

Zaire ebolavirus isolate Ebola virus H. sapiens-wt/SLE/2014/ManoRiver-G3823, partial genome

| | | | | | |
|---|---|---|---|---|---|
| 102 | 102 | 100% | 1e-23 | 100% | KM233097.1 |

Zaire ebolavirus isolate Ebola virus H. sapiens-wt/SLE/2014/ManoRiver-G3822, partial genome

| | | | | | |
|---|---|---|---|---|---|
| 102 | 102 | 100% | 1e-23 | 100% | KM233096.1 |

Zaire ebolavirus isolate Ebola virus H. sapiens-wt/SLE/2014/ManoRiver-G3821, partial genome

| | | | | | |
|---|---|---|---|---|---|
| 102 | 102 | 100% | 1e-23 | 100% | KM233095.1 |

Zaire ebolavirus isolate Ebola virus H. sapiens-wt/SLE/2014/ManoRiver-G3819, partial genome

| | | | | | |
|---|---|---|---|---|---|
| 102 | 102 | 100% | 1e-23 | 100% | KM233093.1 |

Zaire ebolavirus isolate Ebola virus H. sapiens-wt/SLE/2014/ManoRiver-G3818, partial genome

| | | | | | |
|---|---|---|---|---|---|
| 102 | 102 | 100% | 1e-23 | 100% | KM233092.1 |

Zaire ebolavirus isolate Ebola virus H. sapiens-wt/SLE/2014/ManoRiver-G3817, partial genome

| | | | | | |
|---|---|---|---|---|---|
| 102 | 102 | 100% | 1e-23 | 100% | KM233091.1 |

Zaire ebolavirus isolate Ebola virus H. sapiens-wt/SLE/2014/ManoRiver-G3816, partial genome

| | | | | | |
|---|---|---|---|---|---|
| 102 | 102 | 100% | 1e-23 | 100% | KM233090.1 |

Zaire ebolavirus isolate Ebola virus H. sapiens-wt/SLE/2014/ManoRiver-G3814, partial genome

| | | | | | |
|---|---|---|---|---|---|
| 102 | 102 | 100% | 1e-23 | 100% | KM233089.1 |

Zaire ebolavirus isolate Ebola virus H. sapiens-wt/SLE/2014/ManoRiver-G3810.2, partial genome

| | | | | | |
|---|---|---|---|---|---|
| 102 | 102 | 100% | 1e-23 | 100% | KM233088.1 |

Zaire ebolavirus isolate Ebola virus H. sapiens-wt/SLE/2014/ManoRiver-G3810.1, partial genome

| | | | | | |
|---|---|---|---|---|---|
| 102 | 102 | 100% | 1e-23 | 100% | KM233087.1 |

Zaire ebolavirus isolate Ebola virus H. sapiens-wt/SLE/2014/ManoRiver-G3809, partial genome

| | | | | | |
|---|---|---|---|---|---|
| 102 | 102 | 100% | 1e-23 | 100% | KM233086.1 |

Zaire ebolavirus isolate Ebola virus H. sapiens-wt/SLE/2014/ManoRiver-G3808, partial genome

| | | | | | |
|---|---|---|---|---|---|
| 102 | 102 | 100% | 1e-23 | 100% | KM233085.1 |

Zaire ebolavirus isolate Ebola virus H. sapiens-wt/SLE/2014/ManoRiver-G3807, partial genome

| | | | | | |
|---|---|---|---|---|---|
| 102 | 102 | 100% | 1e-23 | 100% | KM233084.1 |

Zaire ebolavirus isolate Ebola virus H. sapiens-wt/SLE/2014/ManoRiver-G3805.2, partial genome

| | | | | | |
|---|---|---|---|---|---|
| 102 | 102 | 100% | 1e-23 | 100% | KM233083.1 |

Zaire ebolavirus isolate Ebola virus H. sapiens-wt/SLE/2014/ManoRiver-G3805.1, partial genome

| | | | | | |
|---|---|---|---|---|---|
| 102 | 102 | 100% | 1e-23 | 100% | KM233082.1 |

Zaire ebolavirus isolate Ebola virus H. sapiens-wt/SLE/2014/ManoRiver-G3800, partial genome

| | | | | | |
|---|---|---|---|---|---|
| 102 | 102 | 100% | 1e-23 | 100% | KM233081.1 |

Zaire ebolavirus isolate Ebola virus H. sapiens-wt/SLE/2014/ManoRiver-G3799, partial genome

| | | | | | |
|---|---|---|---|---|---|
| 102 | 102 | 100% | 1e-23 | 100% | KM233080.1 |

Zaire ebolavirus isolate Ebola virus H. sapiens-wt/SLE/2014/ManoRiver-G3798, partial genome

| | | | | | |
|---|---|---|---|---|---|
| 102 | 102 | 100% | 1e-23 | 100% | KM233079.1 |

Zaire ebolavirus isolate Ebola virus H. sapiens-wt/SLE/2014/ManoRiver-G3795, partial genome

| | | | | | |
|---|---|---|---|---|---|
| 102 | 102 | 100% | 1e-23 | 100% | KM233077.1 |

Zaire ebolavirus isolate Ebola virus H. sapiens-wt/SLE/2014/ManoRiver-G3789.1, partial genome

| | | | | | |
|---|---|---|---|---|---|
| 102 | 102 | 100% | 1e-23 | 100% | KM233076.1 |

Zaire ebolavirus isolate Ebola virus H. sapiens-wt/SLE/2014/ManoRiver-G3788, partial genome

| | | | | | |
|---|---|---|---|---|---|
| 102 | 102 | 100% | 1e-23 | 100% | KM233075.1 |

Zaire ebolavirus isolate Ebola virus H. sapiens-wt/SLE/2014/ManoRiver-G3787, partial genome

| | | | | | |
|---|---|---|---|---|---|
| 102 | 102 | 100% | 1e-23 | 100% | KM233074.1 |

Zaire ebolavirus isolate Ebola virus H. sapiens-wt/SLE/2014/ManoRiver-G3786, partial genome

| | | | | | |
|---|---|---|---|---|---|
| 102 | 102 | 100% | 1e-23 | 100% | KM233073.1 |

Zaire ebolavirus isolate Ebola virus H. sapiens-wt/SLE/2014/ManoRiver-G3782, partial genome

| | | | | | |
|---|---|---|---|---|---|
| 102 | 102 | 100% | 1e-23 | 100% | KM233072.1 |

Zaire ebolavirus isolate Ebola virus H. sapiens-wt/SLE/2014/ManoRiver-G3771, partial genome

| | | | | | |
|---|---|---|---|---|---|
| 102 | 102 | 100% | 1e-23 | 100% | KM233071.1 |

Zaire ebolavirus isolate Ebola virus H. sapiens-wt/SLE/2014/ManoRiver-G3770.2, partial genome

| | | | | | |
|---|---|---|---|---|---|
| 102 | 102 | 100% | 1e-23 | 100% | KM233070.1 |

Zaire ebolavirus isolate Ebola virus H. sapiens-wt/SLE/2014/ManoRiver-G3770.1, partial genome

| | | | | | |
|---|---|---|---|---|---|
| 102 | 102 | 100% | 1e-23 | 100% | KM233069.1 |

Zaire ebolavirus isolate Ebola virus H. sapiens-wt/SLE/2014/ManoRiver-G3769.3, partial genome

| | | | | | |
|---|---|---|---|---|---|
| 102 | 102 | 100% | 1e-23 | 100% | KM233067.1 |

Zaire ebolavirus isolate Ebola virus H. sapiens-wt/SLE/2014/ManoRiver-G3769.2, partial genome

| | | | | | |
|---|---|---|---|---|---|
| 102 | 102 | 100% | 1e-23 | 100% | KM233066.1 |

Zaire ebolavirus isolate Ebola virus H. sapiens-wt/SLE/2014/ManoRiver-G3769.1, partial genome

| | | | | | |
|---|---|---|---|---|---|
| 102 | 102 | 100% | 1e-23 | 100% | KM233065.1 |

Zaire ebolavirus isolate Ebola virus H. sapiens-wt/SLE/2014/ManoRiver-G3765.2, partial genome

| | | | | | |
|---|---|---|---|---|---|
| 102 | 102 | 100% | 1e-23 | 100% | KM233064.1 |

Zaire ebolavirus isolate Ebola virus H. sapiens-wt/SLE/2014/ManoRiver-G3764, partial genome

| | | | | | |
|---|---|---|---|---|---|
| 102 | 102 | 100% | 1e-23 | 100% | KM233063.1 |

Zaire ebolavirus isolate Ebola virus H. sapiens-wt/SLE/2014/ManoRiver-G3758, partial genome

| | | | | | |
|---|---|---|---|---|---|
| 102 | 102 | 100% | 1e-23 | 100% | KM233062.1 |

Zaire ebolavirus isolate Ebola virus H. sapiens-wt/SLE/2014/ManoRiver-G3752, partial genome

| | | | | | |
|---|---|---|---|---|---|
| 102 | 102 | 100% | 1e-23 | 100% | KM233061.1 |

Zaire ebolavirus isolate Ebola virus H. sapiens-wt/SLE/2014/ManoRiver-G3750.3, partial genome

| | | | | | |
|---|---|---|---|---|---|
| 102 | 102 | 100% | 1e-23 | 100% | KM233060.1 |

Zaire ebolavirus isolate Ebola virus H. sapiens-wt/SLE/2014/ManoRiver-G3750.2, partial genome

| | | | | | |
|---|---|---|---|---|---|
| 102 | 102 | 100% | 1e-23 | 100% | KM233059.1 |

Zaire ebolavirus isolate Ebola virus H. sapiens-wt/SLE/2014/ManoRiver-G3750.1, partial genome

| | | | | | |
|---|---|---|---|---|---|
| 102 | 102 | 100% | 1e-23 | 100% | KM233058.1 |

Zaire ebolavirus isolate Ebola virus H. sapiens-wt/SLE/2014/ManoRiver-G3735.2, partial genome

| | | | | | |
|---|---|---|---|---|---|
| 102 | 102 | 100% | 1e-23 | 100% | KM233057.1 |

Zaire ebolavirus isolate Ebola virus H. sapiens-wt/SLE/2014/ManoRiver-G3735.1, partial genome

| | | | | | |
|---|---|---|---|---|---|
| 102 | 102 | 100% | 1e-23 | 100% | KM233056.1 |

Zaire ebolavirus isolate Ebola virus H. sapiens-wt/SLE/2014/ManoRiver-G3734.1, partial genome

| | | | | | |
|---|---|---|---|---|---|
| 102 | 102 | 100% | 1e-23 | 100% | KM233055.1 |

Zaire ebolavirus isolate Ebola virus H. sapiens-wt/SLE/2014/ManoRiver-G3729, partial genome

| | | | | | |
|---|---|---|---|---|---|
| 102 | 102 | 100% | 1e-23 | 100% | KM233054.1 |

Zaire ebolavirus isolate Ebola virus H. sapiens-wt/SLE/2014/ManoRiver-G3724, partial genome

| | | | | | |
|---|---|---|---|---|---|
| 102 | 102 | 100% | 1e-23 | 100% | KM233053.1 |

Zaire ebolavirus isolate Ebola virus H. sapiens-wt/SLE/2014/ManoRiver-G3713.4, partial genome

| | | | | | |
|---|---|---|---|---|---|
| 102 | 102 | 100% | 1e-23 | 100% | KM233052.1 |

Zaire ebolavirus isolate Ebola virus H. sapiens-wt/SLE/2014/ManoRiver-G3713.3, partial genome

| | | | | | |
|---|---|---|---|---|---|
| 102 | 102 | 100% | 1e-23 | 100% | KM233051.1 |

Zaire ebolavirus isolate Ebola virus H. sapiens-wt/SLE/2014/ManoRiver-G3713.2, partial genome

| | | | | | |
|---|---|---|---|---|---|
| 102 | 102 | 100% | 1e-23 | 100% | KM233050.1 |

Zaire ebolavirus isolate Ebola virus H. sapiens-wt/SLE/2014/ManoRiver-G3707, partial genome

| | | | | | |
|---|---|---|---|---|---|
| 102 | 102 | 100% | 1e-23 | 100% | KM233049.1 |

Zaire ebolavirus isolate Ebola virus H. sapiens-wt/SLE/2014/ManoRiver-EM124.4, partial genome

| | | | | | |
|---|---|---|---|---|---|
| 102 | 102 | 100% | 1e-23 | 100% | KM233048.1 |

Zaire ebolavirus isolate Ebola virus H. sapiens-wt/SLE/2014/ManoRiver-EM124.3, partial genome

| | | | | | |
|---|---|---|---|---|---|
| 102 | 102 | 100% | 1e-23 | 100% | KM233047.1 |

Zaire ebolavirus isolate Ebola virus H. sapiens-wt/SLE/2014/ManoRiver-EM124.2, partial genome

| | | | | | |
|---|---|---|---|---|---|
| 102 | 102 | 100% | 1e-23 | 100% | KM233046.1 |

Zaire ebolavirus isolate Ebola virus H. sapiens-wt/SLE/2014/ManoRiver-EM124.1, partial genome

| | | | | | |
|---|---|---|---|---|---|
| 102 | 102 | 100% | 1e-23 | 100% | KM233045.1 |

Zaire ebolavirus isolate Ebola virus H. sapiens-wt/SLE/2014/ManoRiver-EM121, partial genome

| | | | | | |
|---|---|---|---|---|---|
| 102 | 102 | 100% | 1e-23 | 100% | KM233044.1 |

Zaire ebolavirus isolate Ebola virus H. sapiens-wt/SLE/2014/ManoRiver-EM120, partial genome

| | | | | | |
|---|---|---|---|---|---|
| 102 | 102 | 100% | 1e-23 | 100% | KM233043.1 |

Zaire ebolavirus isolate Ebola virus H. sapiens-wt/SLE/2014/ManoRiver-EM119, partial genome

| | | | | | |
|---|---|---|---|---|---|
| 102 | 102 | 100% | 1e-23 | 100% | KM233042.1 |

Zaire ebolavirus isolate Ebola virus H. sapiens-wt/SLE/2014/ManoRiver-EM115, partial genome

| | | | | | |
|---|---|---|---|---|---|
| 102 | 102 | 100% | 1e-23 | 100% | KM233041.1 |

Zaire ebolavirus isolate Ebola virus H. sapiens-wt/SLE/2014/ManoRiver-EM113, partial genome

| | | | | | |
|---|---|---|---|---|---|
| 102 | 102 | 100% | 1e-23 | 100% | KM233040.1 |

Zaire ebolavirus isolate Ebola virus H. sapiens-wt/SLE/2014/ManoRiver-EM112, partial genome

| | | | | | |
|---|---|---|---|---|---|
| 102 | 102 | 100% | 1e-23 | 100% | KM233039.1 |

Zaire ebolavirus isolate Ebola virus H. sapiens-wt/SLE/2014/ManoRiver-EM111, partial genome

| | | | | | |
|---|---|---|---|---|---|
| 102 | 102 | 100% | 1e-23 | 100% | KM233038.1 |

Zaire ebolavirus isolate Ebola virus H. sapiens-wt/SLE/2014/ManoRiver-EM110, partial genome

| | | | | | |
|---|---|---|---|---|---|
| 102 | 102 | 100% | 1e-23 | 100% | KM233037.1 |

Zaire ebolavirus isolate Ebola virus H. sapiens-wt/SLE/2014/ManoRiver-EM106, partial genome

| | | | | | |
|---|---|---|---|---|---|
| 102 | 102 | 100% | 1e-23 | 100% | KM233036.1 |

Zaire ebolavirus isolate Ebola virus H. sapiens-wt/SLE/2014/ManoRiver-EM104, partial genome

| | | | | | |
|---|---|---|---|---|---|
| 102 | 102 | 100% | 1e-23 | 100% | KM233035.1 |

Zaire ebolavirus isolate Ebola virus H. sapiens-wt/SLE/2014/ManoRiver-G3687.1, partial genome

| | | | | | |
|---|---|---|---|---|---|
| 102 | 102 | 100% | 1e-23 | 100% | KM034563.1 |

Zaire ebolavirus isolate Ebola virus H. sapiens-wt/SLE/2014/ManoRiver-G3686.1, partial genome

| | | | | | |
|---|---|---|---|---|---|
| 102 | 102 | 100% | 1e-23 | 100% | KM034562.1 |

Zaire ebolavirus isolate Ebola virus H. sapiens-wt/SLE/2014/ManoRiver-G3683.1, partial genome

| | | | | | |
|---|---|---|---|---|---|
| 102 | 102 | 100% | 1e-23 | 100% | KM034561.1 |

Zaire ebolavirus isolate Ebola virus H. sapiens-wt/SLE/2014/ManoRiver-G3682.1, partial genome

| | | | | | |
|---|---|---|---|---|---|
| 102 | 102 | 100% | 1e-23 | 100% | KM034560.1 |

Zaire ebolavirus isolate Ebola virus H. sapiens-wt/SLE/2014/ManoRiver-G3680.1, partial genome

| | | | | | |
|---|---|---|---|---|---|
| 102 | 102 | 100% | 1e-23 | 100% | KM034559.1 |

Zaire ebolavirus isolate Ebola virus H. sapiens-wt/SLE/2014/ManoRiver-G3679.1, partial genome

| | | | | | |
|---|---|---|---|---|---|
| 102 | 102 | 100% | 1e-23 | 100% | KM034558.1 |

Zaire ebolavirus isolate Ebola virus H. sapiens-wt/SLE/2014/ManoRiver-G3677.2, partial genome

| | | | | | |
|---|---|---|---|---|---|
| 102 | 102 | 100% | 1e-23 | 100% | KM034557.1 |

Zaire ebolavirus isolate Ebola virus H. sapiens-wt/SLE/2014/ManoRiver-G3677.1, partial genome

| | | | | | |
|---|---|---|---|---|---|
| 102 | 102 | 100% | 1e-23 | 100% | KM034556.1 |

Zaire ebolavirus isolate Ebola virus H. sapiens-wt/SLE/2014/ManoRiver-G3676.2, partial genome

| | | | | | |
|---|---|---|---|---|---|
| 102 | 102 | 100% | 1e-23 | 100% | KM034555.1 |

Zaire ebolavirus isolate Ebola virus H. sapiens-wt/SLE/2014/ManoRiver-G3676.1, partial genome

| | | | | | |
|---|---|---|---|---|---|
| 102 | 102 | 100% | 1e-23 | 100% | KM034554.1 |

Zaire ebolavirus isolate Ebola virus H. sapiens-wt/SLE/2014/ManoRiver-G3670.1, partial genome

| | | | | | |
|---|---|---|---|---|---|
| 102 | 102 | 100% | 1e-23 | 100% | KM034553.1 |

Zaire ebolavirus isolate Ebola virus H. sapiens-wt/SLE/2014/ManoRiver-EM098, partial genome

| | | | | | |
|---|---|---|---|---|---|
| 102 | 102 | 100% | 1e-23 | 100% | KM034552.1 |

Zaire ebolavirus isolate Ebola virus H. sapiens-wt/SLE/2014/ManoRiver-EM096, partial genome

| | | | | | |
|---|---|---|---|---|---|
| 102 | 102 | 100% | 1e-23 | 100% | KM034551.1 |

Zaire ebolavirus isolate Ebola virus H. sapiens-wt/SLE/2014/ManoRiver-EM095, partial genome

| | | | | | |
|---|---|---|---|---|---|
| 102 | 102 | 100% | 1e-23 | 100% | KM034550.1 |

Zaire ebolavirus isolate H. sapiens-wt/SLE/2014/ManoRiver-EM095B, partial genome

| | | | | | |
|---|---|---|---|---|---|
| 102 | 102 | 100% | 1e-23 | 100% | KM034549.1 |

Mutant Zaire ebolavirus, complete sequence

| | | | | |
|---|---|---|---|---|
| 102 | 102 | 100% | 1e-23 | 100% |

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

This application may be related to International Patent Application No. PCT/US2013/035750, filed Apr. 9, 2013, which claims the benefit of U.S. Provisional Application No. 61/621,975, filed Apr. 9, 2012, the entire contents of which are incorporated herein by reference.

This application may be related to International Patent Application No. PCT/US2011/047049, filed Aug. 9, 2011, which claims the benefit of U.S. Provisional Application No. 61/373,695, filed Aug. 13, 2010, the entire contents of which are incorporated herein by reference.

All patents and publications mentioned in this specification are herein incorporated by reference to the same extent as if each independent patent and publication was specifically and individually indicated to be incorporated by reference.

What is claimed is:

1. A method of detecting a specific target polynucleotide in an isothermal amplification reaction coupled with reverse transcription, the method comprising:
(a) contacting a target polynucleotide molecule in a sample with a primer in the presence of a reverse transcriptase and dNTPs under conditions permissive for cDNA synthesis, thereby generating a cDNA;
(b) contacting the cDNA with forward and reverse primers each carrying at least one nicking enzyme recognition sequence within their respective 5′-terminal regions which specifically bind the cDNA with their respective 3′-terminal regions in the presence of a nicking enzyme, dNTPs, a detectable oligonucleotide probe, and a strand-displacement polymerase under conditions permissive for the isothermal amplification of the cDNA; and
(c) detecting a signal specific for detectable oligonucleotide probe hybridization to the amplicon, wherein detection of the signal indicates the presence or quantity of the target polynucleotide present in the sample and failure to detect the signal indicates the absence of target polyribonucleotide in the sample.

2. A method of detecting an RNA virus in a sample, the method comprising
(a) contacting an RNA virus polynucleotide molecule in a sample with a primer in the presence of a reverse transcriptase and dNTPs under conditions permissive for cDNA synthesis, thereby generating a cDNA;
(b) contacting the cDNA with forward and reverse primers each carrying at least one nicking enzyme recognition sequence within their respective 5′-terminal regions which specifically bind the cDNA with their respective 3′-terminal regions in the presence of a nicking enzyme, dNTPs, a detectable oligonucleotide probe, and a strand-displacement polymerase under conditions permissive for the isothermal amplification of the cDNA; and
(c) detecting a signal specific for detectable oligonucleotide probe hybridization to the amplicon, wherein detection of the signal indicates the presence or quantity of the RNA virus polynucleotide molecule present in the sample and failure to detect the amplicon indicates the absence of an RNA virus.

3. The method of claim 1 or 2, wherein the polynucleotide molecule is an Ebola, HIV, dengue, influenza, bovine diarrhea virus, yellow fever virus, west nile virus, hepatitis C, Lassa virus, Flavivirus, or Arenavirus polynucleotide.

4. A method of detecting an Ebola virus in a sample, the method comprising
(a) contacting a Ebola polynucleotide in a sample with a primer in the presence of a reverse transcriptase and dNTPs under conditions permissive for cDNA synthesis, thereby generating a cDNA;
(b) contacting the cDNA with forward and reverse primers each carrying at least one nicking enzyme recognition sequence within their respective 5′-terminal regions which specifically bind the cDNA with their respective 3′-terminal regions in the presence of a nicking enzyme, dNTPs, a detectable oligonucleotide probe, and a strand-displacement polymerase under conditions permissive for the isothermal amplification of the cDNA; and
(c) detecting a signal specific for detectable oligonucleotide probe hybridization to the amplicon, wherein detection of the signal indicates the presence or quantity of the Ebola polynucleotide present in the sample and failure to detect the signal indicates the absence of Ebola polynucleotide present in the sample.

5. A method of detecting an Ebola virus in a sample, the method comprising
(a) contacting a biological sample with an agent capable of extracting an polynucleotide molecule present in the sample and an agent capable of stabilizing an polynucleotide molecule against degradation;
(b) contacting the extracted and stabilized Ebola RNA with a primer in the presence of a reverse transcriptase and dNTPs under conditions permissive for cDNA synthesis, thereby generating an Ebola cDNA;
(c) contacting the Ebola cDNA with forward and reverse primers each carrying at least one nicking enzyme recognition sequence within their respective 5′-terminal regions which specifically bind the Ebola cDNA with their respective 3′-terminal regions in the presence of a nicking enzyme, dNTPs, and a strand-displacement polymerase under conditions permissive for the isothermal amplification of the cDNA, thereby generating amplicons; and
(d) detecting the amplicons, wherein the presence of an Ebola amplicon detects an Ebola polynucleotide molecule in the sample and failure to detect the amplicon indicates the absence of an Ebola polynucleotide in the sample.

6. The method of any one of claims 1-5, wherein the polynucleotide molecule is obtained by contacting the sample with one or more of an agent capable of extracting an RNA molecule present in the sample and an agent capable of stabilizing an RNA molecule against degradation.

7. The method of any one of claims 1-6, wherein no detectable signal is present in a control assay lacking a target polynucleotide at seven minutes, ten minutes, and/or fifteen minutes following initiation of the assay.

8. The method of any one of claims 1-7, wherein the primer used in step (a) has the same sequence or a different sequence than a primer used in step (b).

9. The method of any one of claims 1-8, wherein steps (a)-(c) are carried out in a single reaction.

10. The method of any one of claims 1-9, wherein the reverse transcriptase enzyme and the strand-displacement DNA polymerase are the same or different enzymes.

11. The method of any one of claims 1-8, wherein the cDNA of step (a) is generated in a first reaction vessel, then transferred to a second reaction vessel where step (b) is carried out.

12. The method of any one of claims 1-11, wherein the sample is a bodily fluid.

13. The method of claim 12, wherein the bodily fluid is selected from the group consisting of saliva, sweat, tears, fluids accumulating in a bodily cavity, urine, ejaculate, vaginal secretion, cerebrospinal fluid, lymph, feces, sputum, decomposition fluid, vomit, sweat, breast milk, blood, serum, and plasma.

14. The method of claim 13, wherein the bodily cavity is peritoneal cavity or pericardial cavity.

15. The method of any one of claims 1-14, wherein the limit of detection is 10 or 20 copies per reaction.

16. The method of any one of claims 1-15, wherein the method is carried out in about 5, 7, 10, 15, 20, 25 or thirty minutes.

17. The method of any one of claims 1-16, wherein steps (a)-(d) are carried out in the context of the biological sample.

18. The method of any one of claims 3-17, wherein Ebola RNA is not purified or isolated away from the biological sample.

19. The method of any one of claims 1-18, wherein the method is carried out at a point of care or diagnosis in a portable battery powered device.

20. The method of any one of claims 1-19, wherein no separate reverse transcriptase primer is required, but the forward and/or reverse primers are used.

21. The method of any one of claims 1-20, wherein the sample is a biological sample or an environmental sample.

22. The method of claim 21, wherein the biological sample is obtained from a subject, bat, bush meat, or a domestic animal.

23. The method of claim 21, wherein the biological sample is a swab of a mucosal membrane selected from the group consisting of buccal, nasal, eye, rectal, and vaginal or skin.

24. The method of claim 21, wherein the biological sample is a tissue sample obtained from a subject, necropsy, or culture media.

25. The method of claim 24, wherein the necropsy is of a human, primate, bat, or other mammal.

26. The method of claim 21, wherein the environmental sample is a material that may be contaminated with a biological fluid of a subject having or having a propensity to develop an Ebola viral infection.

27. The method of claim 26, wherein the environmental sample is bedding, a seat cushion, a rug, an air condition filter or other material.

28. The method of any one of claims 1-27, wherein the polymerase are 5′-exo derivatives of Bst DNA polymerase I, Gst DNA polymerase I, Gka DNA polymerase I, Gca DNA polymerase I, Gan DNA polymerase I, Gbo DNA polymerase I, Gsp70 DNA polymerase I, GspT3 DNA polymerase I, Gsp52 DNA polymerase I and/or fragments thereof.

29. The method of any one of claims 1-28, wherein the nicking enzyme is one or more of Nt.BstNBI, Nt.BspD6I, Nt.BspQI, Nt.BsmAI, Nt.AlwI, N.Bst9I, or N.BstSEI.

30. The method of any one of claims 1-29, wherein the reverse transcriptase is M-MLV RT, AMV RT, RSV RT, and/or mutants/derivatives thereof.

31. The method of any one of claims 1-30, wherein the detectable probe comprises a molecular beacon.

32. The method of any one of claims 3-31, wherein the forward and reverse primers for detection of EBOV comprise the following sequences, respectively:
Forward primer:
(SEQ ID NO: 1)
GACTCGATATCGAGTCGCTTCCA[MeOC]AGTTATC[MeOU][MeOA]
[MeOC][MeOC][MeOG]
Reverse Primer:
(SEQ ID NO: 2)
GACTCGATATCGAGTCGAAATGC[MeOA]ACGA[MeOC][MeOA][MeOC]
[MeOC[MeOU]

33. The method of any one of claims 3-32, wherein amplification of EBOV is detected using a probe having the following sequence: gctacACGACTTTYGCTGAAGgtagc.

34. The method of claim 33, wherein the probe has a fluorescent dye at the 5′ end, and a quencher at the 3′ end or a fluorescent dye at the 3′ end, and a quencher at 5′ end.

35. The method of claim 34, wherein the probe is
(SEQ ID NO: 4)
5′-CALRed_{610 nm}-gctacACGACTTTYGCTGAAGgtagc BHQ2-3′
or
(SEQ ID NO: 5)
5′-FAM or FITC-gctacACGACTTTYGCTGAAGgtagc-BHQ1-3′

36. The method of claim 35, wherein the 3′ quencher is replaced by DABsyl.

37. The method of any one of claims 3-31, wherein the forward and reverse primers for detection of HIV comprise one or more of the following sequences, respectively:
Forward primers:
GACTCGATATCGAGTCTGACTAGmCGGAGGmCmTmAmGmAmAmG,
GACTCGATATCGAGTCTGACTAGmCAGAGGmCmTmAmGmAmAmG;
and
Reverse Primers:
GACTCGATATCGAGTCTATTGACmGCTCmTmCmGmCmAmC,
GACTCGATATCGAGTCTACTGACmGCTCmTmCmGmCmAmC.

38. The method of any one of claims 3-31 and 37, wherein amplification of HIV is detected using a probe having the following sequence: cgcaagGGAGAGAGATGGGTGcttgcg.

39. The method of any one of claims 3-31, wherein the forward and reverse primers for detection of Dengue comprise one or more of the following sequences, respectively:
Forward primer:
GACTCGATATCGAGTCCAAAAACmAGCATATTmGmAmCmGmC,
and
Reverse Primer:
GACTCGATATCGAGTCAGACAGCmAGGATCmTmCmTmGmG,
GACTCGATATCGAGTCAGACAGCmAGGATCmTmGmTmGmG.

40. The method of any one of claims 3-31 and 39, wherein amplification of Dengue is detected using a probe having the following sequence: cgcatcTGGTCTTTCCCAGCgatgcg.

41. The method of any one of claims 3-31, wherein the forward and reverse primers for detection of influenza B comprise one or more of the following sequences, respectively:
Forward primers:
GACTCGATATCGAGTCAAATGCAmGATGGTCTCmAmGmCmTmA,
GACTCGATATCGAGTCAAATGCAmAATGGTCTCmAmGmCmTmA,
GACTCGATATCGAGTCAAATGCAmGATGGTTTCmAmGmCmTmA;
and
Reverse Primers:
GACTCGATATCGAGTCCTCCTTTmTCCCATTCCATmTmCmAmTmT,
GACTCGATATCGAGTCCTCCCTTmTCCCATTCCATmTmCmAmTmT,
GACTCGATATCGAGTCCTCCTTTmCCCCATTCCATmTmCmAmTmT.

42. The method of any one of claims 3-31 and 41, wherein amplification of influenza B is detected using a probe having the following sequence: gccaaGCTATGAACACAGCAAActtggc.

43. The method of any one of claims 3-31, wherein the forward and reverse primers for detection of BVDV1 comprise one or more of the following sequences, respectively:
Forward primers:
GACTCGATATCGAGTCGGCCCACmTGTATTGCTmAmCmTmGmAmAmA,
GACTCGATATCGAGTCGGCCCACmTGCACTGCTmAmCmTmAmAmAmA;
and
Reverse Primer:
GACTCGATATCGAGTCTGTGATCmAACTCCmAmTmGmTmGmCmC.

44. The method of any one of claims 3-31 and 43, wherein amplification of BVDV1 is detected using a probe having the following sequence: cgctacATCTCTGCTGTACATGgtagcg.

45. The method of any one of claims 1-44, wherein the probe has a fluorescent dye at the 5′ end and a quencher at the 3′ end, or a fluorescent dye at the 3′ end and a quencher at the 5′ end.

46. The method of claim 45, wherein the fluorescent dye is CALRed₆₁₀ₙₘ, and the quencher is BHQ2 or DABsyl.

47. The method of claim 45, wherein the fluorescent dye is FAM or FITC and the quencher is BHQ1 or DABsyl.

48. The method of any one of claims 1-47, wherein a forward or reverse primer comprises at the 3′ end one or more 2′ modified nucleotides.

49. The method of claim 48, wherein the one or more 2′ modified nucleotide is one or more of 2′-O-methyl, 2′-methoxyethoxy, 2′-fluoro, 2′-hydroxyl, 2′-alkyl, 2′-allyl, 2′-O-[2-(methylamino)-2-oxoethyl], 4′-thio, 4′-CH₂—O-2′-bridge, 4′-(CH₂)₂-O-2′-bridge, 2′-LNA, and 2′-O—(N-methylcarbamate).

50. A kit for detecting an RNA virus polynucleotide molecule comprising primers that specifically bind an RNA viral sequence, a detectable probe that specifically binds an RNA virus amplicon, a reverse transcriptase enzyme, a nicking enzyme, and a strand-displacement polymerase.

51. The kit of claim 50, for the detection of EBOV, wherein the primers comprise the following sequences:
Forward primer:
GACTCGATATCGAGTCGCTTCCA[MeOC]AGTTATC[MeOU][MeOA]
[MeOC][MeOC][MeOG]
Reverse Primer:
GACTCGATATCGAGTCGAAATGC[MeOA]ACGA[MeOC][MeOA]
[MeOC][MeOC][MeOU].

52. The kit of claim 50, wherein the probe comprises the following sequence:
gctacACGACTTTYGCTGAAGgtagc.

53. The kit of claim 52, wherein the probe has a fluorescent dye at the 5′ end and a quencher at the 3′ end or fluorescent dye at the 3′ end and a quencher at the 5′ end.

54. The kit of claim 53, wherein the probe is
5′-CALRed₆₁₀ₙₘ-gctacACGACTTTYGCTGAAGgtagc BHQ2-3′
or
5′-FAM or FITC-gctacACGACTTTYGCTGAAGgtagc-BHQ1-3′.

55. The kit of claim 54, wherein the 3′ quencher is replaced by DABsyl.

56. The kit of claim 50, for detection of HIV, wherein the primers comprise one or more of the following sequences, respectively:
Forward primers:
GACTCGATATCGAGTCTGACTAGmCGGAGGmCmTmAmGmAmAmG,
GACTCGATATCGAGTCTGACTAGmCAGAGGmCmTmAmGmAmAmG;
and
Reverse Primers:
GACTCGATATCGAGTCTATTGACmGCTCmTmCmGmCmAmC,
GACTCGATATCGAGTCTACTGACmGCTCmTmCmGmCmAmC.

57. The kit of claim 50, wherein the probe comprises the following sequence:
cgcaagGGAGAGAGATGGGTGcttgcg.

58. The kit of claim 50, for detection of Dengue, wherein the primers comprise one or more of the following sequences, respectively:
Forward primer:
GACTCGATATCGAGTCCAAAAACmAGCATATTmGmAmCmGmC,
and
Reverse Primer:
GACTCGATATCGAGTCAGACAGCmAGGATCmTmCmTmGmG,
and
GACTCGATATCGAGTCAGACAGCmAGGATCmTmGmTmGmG.

59. The kit of claim 50, for detection of Dengue, wherein the probe comprises the following sequence: cgcatcTGGTCTTTCCCAGCgatgcg.

60. The kit of claim 50, for detection of influenza B, wherein the primers comprise one or more of the following sequences, respectively:
Forward primers:
GACTCGATATCGAGTCAAATGCAmGATGGTCTCmAmGmCmTmA,
GACTCGATATCGAGTCAAATGCAmAATGGTCTCmAmGmCmTmA,
GACTCGATATCGAGTCAAATGCAmGATGGTTTCmAmGmCmTmA;
and
Reverse Primers:
GACTCGATATCGAGTCCTCCTTTmTCCCATTCCATmTmCmAmTmT,
GACTCGATATCGAGTCCTCCCTTmTCCCATTCCATmTmCmAmTmT,
(SEQ ID NO: 20)
GACTCGATATCGAGTCCTCCTTTmCCCCATTCCATmTmCmAmTmT.

61. The kit of claim 50, for detection of influenza B, wherein the probe comprises the following sequence: gccaaGCTATGAACACAGCAAActtggc.

62. The kit of claim 50, for detection of BVDV1, wherein the primers comprise one or more of the following sequences, respectively:
Forward primers:
GACTCGATATCGAGTCGGCCCACmTGTATTGCTmAmCmTmGmAmAmA,
GACTCGATATCGAGTCGGCCCACmTGCACTGCTmAmCmTmAmAmAmA;
and
Reverse Primer:
GACTCGATATCGAGTCTGTGATCmAACTCCmAmTmGmTmGmCmC.

63. The kit of claim 50, for detection of BVDV1, wherein the probe comprises the following sequence: cgctacATCTCTGCTGTACATGgtagcg.

64. The method of any one of claims 50, 57, 59, 61 and 63, wherein the probe has a fluorescent dye at the 5′ end and a quencher at the 3′ end, or a fluorescent dye at the 3′ end and a quencher at the 5′ end.

65. The kit of claim 64, wherein the fluorescent dye is CALRed₆₁₀ₙₘ, and the quencher is BHQ2 or DABsyl.

66. The kit of claim 65, wherein the fluorescent dye is FAM or FITC and the quencher is BHQ1 or DABsyl.

67. A kit for amplifying an Ebola polynucleotide molecule in a reverse transcriptase nicking amplification reaction, the kit comprising the following primers:
Forward primer:
GACTCGATATCGAGTCGCTTCCA[MeOC]AGTTATC[MeOU]
[MeOA][MeOC][MeOC][MeOG]
Reverse Primer:
GACTCGATATCGAGTCGAAATGC[MeOA]ACGA[MeOC]
[MeOA][MeOC][MeOC][MeOU];
the following probe:
gctacACGACTTTYGCTGAAGgtagc;
a reverse transcriptase enzyme, a nicking enzyme, a strand-displacement polymerase, and directions for use of the aforementioned primers, probes and enzymes for detecting an Ebola polynucleotide molecule.

68. A kit for amplifying an HIV polynucleotide molecule in a reverse transcriptase nicking amplification reaction, the kit comprising one or more of the following primers:
GACTCGATATCGAGTCTGACTAGmCGGAGGmCmTmAmGmAmAmG,
GACTCGATATCGAGTCTGACTAGmCAGAGGmCmTmAmGmAmAmG;
and
Reverse Primer:
GACTCGATATCGAGTCTATTGACmGCTCmTmCmGmCmAmC,
GACTCGATATCGAGTCTACTGACmGCTCmTmCmGmCmAmC;
the following probe:
cgcaagGGAGAGAGATGGGTGcttgcg;
a reverse transcriptase enzyme, a smoking enzyme, a strand-displacement polymerase, and directions for use of the aforementioned primers, probes and enzymes for detecting an HIV polynucleotide molecule.

69. A kit for amplifying a Dengue polynucleotide molecule in a reverse transcriptase nicking amplification reaction, the kit comprising one or more of the following primers:
Forward primer:
GACTCGATATCGAGTCCAAAAACmAGCATATTmGmAmCmGmC;
and
Reverse Primer:
GACTCGATATCGAGTCAGACAGCmAGGATCmTmCmTmGmG,
GACTCGATATCGAGTCAGACAGCmAGGATCmTmGmTmGmG;
the following probe:
cgcatcTGGTCTTTCCCAGCgatgcg;
a reverse transcriptase enzyme, a nicking enzyme, a strand-displacement polymerase, and directions for use of the aforementioned primers, probes and enzymes for detecting an Dengue polynucleotide molecule.

70. A kit for amplifying an influenza B polynucleotide molecule in a reverse transcriptase nicking amplification reaction, the kit comprising one or more of the following primers:
Forward primer:
GACTCGATATCGAGTCAAATGCAmGATGGTCTCmAmGmCmTmA,
GACTCGATATCGAGTCAAATGCAmAATGGTCTCmAmGmCmTmA,
GACTCGATATCGAGTCAAATGCAmGATGGTTTCmAmGmCmTmA;
and
Reverse Primer:
GACTCGATATCGAGTCCTCCTTTmTCCCATTCCATmTmCmAmTmT,
GACTCGATATCGAGTCCTCCCTTmTCCCATTCCATmTmCmAmTmT,
GACTCGATATCGAGTCCTCCTTTmCCCCATTCCATmTmCmAmTmT;
the following probe:
gccaaGCTATGAACACAGCAAActtggc;
a reverse transcriptase enzyme, a nicking enzyme, a strand-displacement polymerase, and directions for use of the aforementioned primers, probes and enzymes for detecting an influenza B polynucleotide molecule.

71. A kit for amplifying a BVDV1 polynucleotide molecule in a reverse transcriptase nicking amplification reaction, the kit comprising one or more of the following primers:
GACTCGATATCGAGTCGGCCCACmTGTATTGCTmAmCmTmGmAmAmA,
GACTCGATATCGAGTCGGCCCACmTGCACTGCTmAmCmTmAmAmAmA;
and
Reverse Primer:
GACTCGATATCGAGTCTGTGATCmAACTCCmAmTmGmTmGmCmC;
the following probe:
gctacACGACTTTYGCTGAAGgtagc;
a reverse transcriptase enzyme, a nicking enzyme, a strand-displacement polymerase, and directions for use of the aforementioned primers, probes and enzymes for detecting an BVDV1 polynucleotide molecule.

72. The kit of any one of claims 50-71, wherein the kit further comprises a capillary tube that may or may not comprise lyophilized lysis or RNA stabilization reagents for viral polynucleotide extraction.

73. The kit of any one of claims 50-72, wherein the kit further comprises one or more vessels comprising a buffer suitable for carrying out a reverse transcriptase and/or amplification reaction.

74. The kit of any one of claims 50-73, wherein the kit further comprises vessels comprising the reverse transcriptase enzyme, nicking enzyme, and strand-displacement polymerase in lyophilized form.

75. A method of diagnosing a human or animal subject with an RNA virus, the method comprising
(a) contacting a sample of the subject with an agent capable of extracting an RNA virus present in the sample and an agent capable of stabilizing the extracted polynucleotide molecule against degradation;
(b) contacting the polynucleotide molecule with a reverse transcriptase primer in the presence of a reverse transcriptase and dNTPs under conditions permissive for cDNA synthesis, thereby generating a cDNA copy of the polynucleotide molecule;
(c) contacting the cDNA with forward and reverse primers carrying at least one nicking enzyme recognition sequence within their respective 5′-terminal regions which specifically bind the cDNA with their respective 3′-terminal regions in the presence of a nicking enzyme, dNTPs, and a strand-displacement polymerase under conditions permissive for the isothermal amplification of the cDNA, thereby generating amplicons; and
(d) detecting the amplicons, wherein the presence of an RNA viral amplicon diagnoses an RNA viral infection in the subject and failure to detect the amplicon diagnoses the absence of an RNA viral infection in the subject.

76. The method of claim 75, wherein the RNA virus is an Ebola virus, HIV, dengue virus, influenza virus, bovine diarrhea virus, yellow fever virus, west nile virus, hepatitis C virus, Lassa virus, Flavivirus, Arenavirus, or single-stranded RNA virus.

77. The method of claim 75 or 76, wherein the agent capable of extracting the virus is one or a combination of sodium dodecyl sulfate, sodium lauryl sulfate, Guanidinium thiocyanate, and/or guanidine hydrochloride.

78. The method of any one of claims 75-77, wherein the method is used for daily screening of health care workers.

79. The method of any one of claims 77-78, wherein the samples are pooled and the screening is carried out on a human or animal population.
